SEQUENCE LISTING (39 sequences)

SEQ ID NO: 1 - DNA, 150192 bp, Homo sapiens
  source 1..150192: mol_type = genomic DNA
agcttcacagctgggagagcgcaggaaggcgccgggaaggtgagcctcctggactctggggaggtagaaagcaagccaggggaaagaacagttgtcttttagctgataatacaacctagacttgggtctgaaccacctaagacagatttaaagtgtcagaaaaccaggagaggggcggagagggaggactgagactaacgcagtttgctctcgcatcaaactaggaaagccagcccaccagcgtctgggtgggctgcgccgcgcggctggcggaccttcccgggttggagaagtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccaggtaaggagcgtttggaaatgagcgggacttaacgatttggggtgtccaagggatgtttaacagtgcaggtgctataggcaccttgctttttgattatagaaatgtggtttgattgcggtgcttgtggttctcatgtagggtcagctaggtcttagatgctttactttacccttgtcaaagaaaaaagaaaaataggtgtcctgggaagagccaacaacttatggtttcgtattgaagaatcgtttgatttcttgtgacttttactatgttgttcatatcaattttccctcaaaagttttattctccaacttattgggtaggaaaattacggaggttaaagttgtgaattttcccaaagggtgtttattaactgacaaacatataatatcagcatctataagatgtgtcatggttgttctggatattggaaatgagcacagtagtgaatggagttgcttgttaagtcatgttttacagagaatagttttccaacaatcatttatgcatagcagccattcttcacacctattaggaaaaatatgttgtgtgcatagtttccaaaagagtactctttttcatgatgctcttcccaagcctgggtgaaagacatcaagaaaagctgtaacaaattaattccttttctcaaaacattcattttccttgaggtccaagtcaaaaatcaacaatacaattgagattagtaaactatcatacagactttatttggcaaatctacctaagaaaagtttcatatgaaagctttaacctaatactatgtgacatatgcaagaagagaacgcagggtttcacttgccgatcttaaggagaccctgactatttccaggctgtggttaaccggaatcaagttatctatgtaaggctatttgttgaaattattcggtgattgaaggtgaaatgttcaaaataagtcaaagttttacaatgtagttcttaaagctaatctttaaattacagagcgtccgagtttaagtctgtcttaaatgatcatagaaagcaaatattttatgtatttgggccagttgacttaccattaaggacaaagtaggaatgcttttagtattatggaatttataaagtgtaatttctaataggatattgctagcctatgagttcagtagtaaaataactcagctttaaaaagttgagattgctggttatacaccagtattatgagagaaactaagatgattagatttgtaaaacctttacagtttaagttggtattctcagtggatactcatttctactcaatattttaacataactcaaaacatccatcagccaggatttgttctggttcaatgcctctaacctgtagtttattttatgaactatagcccaagaataatatctggtcatattataaaatgttctaagtttattcattggctacatcaacacaataagtaaacaagaaatcttcattataaccagtttataagaacacatagataatcttggagttgttttgtttcatgattctcaaagttaaaaagaaatagctgatataaatacgtcttctgtttctcattttgaaactggaggtaatgattgatggtggatggagagcatttgctctaatcagttgaagaaagggagctaactattagtttagttgtgccatggcaattctgttcttgatgaacctctcagactctgtcaatgttaacatgaatggaaatattatctactgttggtttttattagagtcctagtgaaatggaaataggagaccggtaatggtttcaatgataatctaaatcagtattctcaactcgggctgcaaattagggttaataggagaactttcaaaaaatacggatgctcagccttaccccaattcaatcaaatcagaatatctgtggaaagggtctgcaatttgatgtatttctttaggtgattgtaatatgcaaccaggattgagagccactgaccaagagggtttccaccagacttggtagaactcattgatgtgggaggcttttgataaaaataaatgttgtttgttatatcgctggtcaaatagttttttctttaaaaaaaagaaacatatatgtataaacatacatatatatgtataacttgatcttacaattagaagaaaaatattttaatatttgatgttagatatttagaccgtaacagcttctcggaaaaacattttttatccagaaaacaagctattgagtttgaaattgatctgcccatccagaaaatgttttagtaaatgaactccagctttaagagatgttgaaaataatacttgatgaaacatacaatttcatctccttttgcttaagacctgtatactgtgtaaagtacaaatacgtacccttttgaaagaggagaaagaggaaaatagcatcattccccttggcacactaactaccatatacccggtgccaatgataatctagtacttttacaaaaaatatgttttaagctaagagtagccttgtgagttacaagctattatcattattttatgtatgaggaaattgaggacccattagattaatgaactcgctcaaagccacgaaccaagtaaattaaaaatatatttgaacccaggtctttttaagttcaatgatggattacattatatatttatttacatatttgtattttttacaacttaacagcagcatttatttagagataatataagttaagagtacaaattctggcacagctctgcttgagtttgacttctgattccataactttctatctgtgtgatttgggcacataatgtaacttctcggtgccttaattttctttgtagaataagaattaaatgagttaatatttgtaaagcatttgggtaggtgtctggtacatggtaagttctatgtgttatatttttttaaatgtcattcattcaaaaaaatttttattgttatatgccaagcattgcactaagcctgaagatacatagacaaataagtcatatccctatcctcaaggatgtcgtgtctagtagagaatgacaagaacataagtaatcatcccaatcaggtatagatcttttacttaattaaaagcatcatttcttgacaactgtgtgcttttccatgtgaatgtgatcttccagagcttcatataatgagtgttataaaaccaagtgataagattttgtgttttcataatgatactaacttgttttcaattctagtatcaaaacacagaaaatcatattaacatatgtttctggcttcccttatacatttagaaaaccagcaatatagagaggtttgagatctttaaaattttatattaaaagtgaatcatgaagatagaacattggagaataatcagtagaaaaaatgttatcagtatctgttcctcaatacttctaacagtaattttcttgaattggcttttcaaaaacagttttgtctgattagaatgtagttttggttttgtttttaagaaaaaaaatgtttcagggaagaaagagtggatcacaggatacagaaaaatgtccctaatacaagttggcaaggcaacatatacctcattgaagctggcagcacttcctagtctatgcctgcagctctgcacagctgcctggtacctccagggtaccagtggatacctgaggtataagattctgtctttgttaaaaattaaagtccctgtttattttttaaaaatagcatataaattggtattctttgtaccagaataacacagataatctaaactcaaggagttatttctcaaagaagtaaagtacctggttcagtgaaatatttaactattccatttcactaaaatttatgtttaactatttcagtaaaatttatgtctttcctctctcttatcaagtctcagactacctccagtttttcccatggatgctacatttttcttcctctaagtatcgctgccacacctctagtttaagtttttattatcattaataaaatatttattctcctaatattcagtcacattaagtttctatgtgctaacttcagtgttatagaacatggttagacttgttttctaatctttaatttctccccctctgttcataattattcagtaatggtccaaggactgcaagacttagaacccctcttcagcctggtatttaagatgtttcatggtataatcctagtttagctttcctatccaagtcaggtagaaaacctcaacagctccttaattttagttctgaagctgtagttacaccagaaaaattgactcctgatttggagtccattcttcacttttaaccctgtgcccttgcatgtcaacctcagtctatattcttctgcctcaaaattgaagtgtgtgtgtgtgtgtgtgtgtgtgtgtgtgtgtccctttacatcaatggttccattgtagagctgcttttctttggttttcatgtctcgctgcaaccctagtctcactccctgccctttatttgtccggtggttcagaattcaacatttcaaatcttaccttccttcatccttaaaatgtattaatcattctcccataacacatctaacaaatttccatcctatagctttgctcatattttaaaactgaataaatatgtactaagtgcctattatgtcctagtcaggggatattgtgacagattacagatcctgatttcatggggaatggaaaagacagacaaaccaacaaacattatacagccctgtggtatgtattccaatagagaaatgccatgagggtgcctagccggacaaccagcccggacatcagggaaattttctagggaggttaccctaaagggcatatgattcagcattgaaatgacagaacagaatgagctagataaagaggggaagaaatgaagtgttacaggcaggacaccagattgtggcttgaaacaacatggtgacttggggaaaccacaggtatttttgttgccccagcagggttaaaggacacagattatacacaaagaagcttgcatgcctaccatactaaagggtttgcattttatcaggaagatagtggggggtccttgaaggagttaatataggttactgaaataatcagatttgccattttggtagatctctctggtggaaaataagaggcatattagattgaaaaaaaggatggatacagattaactggacacacgcagccttactgcagtgacagagggaagaatcaattagggcctccttgttggagacagaaaagaaaaaaaaacgaaattattttaaaggtagattttgtaggatttggtgaacgatagaaattagaggtgagatgaaagagaaaaattgtatgatgaattccaggcctctttcatggataataagagagatgtgatgtcattcaccaagtcgggacaaggagtattttggacaggcaagataaatagttgaatttggaggaaaaaaaaataatgtctttggctcatccatgtgatgatgttaagtagaagtgcatatagaaattagtgcctcaagagagaatttggtactagaaatataaatttaagagacatagtgaatctatttcagctgacagcataggaataaagcatattatctctggagaagtaaaaacagggaataaagaaagaaaacatagaggaaaaaattaaaggtagaaaattaaaagaaaagtaaaacatcttagaatcctggaaagcaccaatatttaaagggaaatggaaaacaatcctgcaaaaaaaagacagagatgaaagactacaggagagagacagagagagagagagagagagagagagagagagagagagagagagagaaagtaattttatggaaaccagggaggagaaagcgagcactcatgcgggatgttaccagtagatcaagtgggataaggattgagagctcgcaattgcatttaattgagagcaattttagtcgtgagttgggtgtgggttgagaaataaatgtgaaatgaggaaatggagacactgattgtacacaataatttcaatcagtttgagacgcctgccttactttattttccctttttgaaaggtgcagtctcctaaagcttaacttacatgctgcttccttctttttagcctttcctggacagcgtttaatccccccaacaaagatgtcatatcacatcatgattgatatcccttgttttagagtttccagaggtcccatgcatgtgagctgtgtgcgtttgcatgtgtatgaatatataggtatatattaaatacccatgtagtatgtaaagtatttttctctgttgccatgcattcattgaaataattttcttaatctctttattatgatagtgtatggggagaaaaatgagtttataagcccctgccctcaataataatgatctggaaattctaggatttctgcatataccaaatattttccagactgtttgtgtacttaaaagttgcatagagcaggatgagttcatgtcctttgcagggacatggatgaagctggaaaccatcattctcagcaaactatcacaaggacagtggcatgttctcactcataggtgggaattgaacaatgagaacacttggacacagggcagggaacatcacacactggggcctgttggggggcagggggctgggggagggatagcatcagaagaaatacctaatgtaaatgacgagttgatgagtgcagcaaaccaacatggcacatgtatacctatgtaacaaacctgcacgttgtgcacgtgtaccctagaacttaaagtataattaaaaaaaaaactttttataaaaaaaatttgcatagataaatttttcaggccaaatatagcaatttttgaatcacaaattataacattttatacttgttttatggtggtttaatctcccttttgtattattttcttatttctctatcagattacaaaagacagggtcatcattatttctttgtcccagcacatccaggagaccctctcttctcttagagaaattagttaagacttccctaaatgtgtccataaataaagtgttttttatttctcctgcacactgaggcttaaagttttcttgttttaagttatcaactagctgtttccccagagctttcagctattcatctgtagtacttcctccaaaccaataattcattcatattcttaacatccattcttctattttgggtttgagttggttttctttagtgagcatttaaaacatagcacacatattcagaacatagcattcagtttaatcatgcagaactgtttcccaacagccctctgccctcttaatcacaaagtcatacctctatttctcctctgcttcttgcactaggctggagctacttaggtggtatttatacctcttataggcaagaaaaacaaaactaaactaaacatttttccttctcattttcctaatgtggatccaagccctgaaagcaattaatcctaaattgtttgaatagctgattttccgcaagttaatattaatacctcctagatttcccaggtataactaaaaatagaaaacaaaaacaaaaacaaaaaacagctactattcattgagcccttactgtgtgcaggatacaatgatactcagtgacttcctgagacaatcatatggtatattatcaagcccattctacaggtaaggaaatagggcagaaaacttacagggacccagcaaggttcatacatctctgagtggcagagctggaactcaaacctaagttcgcctaaaaccaaaagactatgctctctctgctgctgtactcaaatggaattttgaaggcgacttagacgtttctttattttaatataattggtatattaattgtcttagatgatcagagcctgtaagtaacagaattaggagcctctctcaatggatatttcagccgatctcttataggctttaaactcagctctccaactcaatcaggccaccggaactgtgactatacctataaccaaattccagccttttggtataacctggaatagttcaggatcacacttcatattaagtttacattccagatagctaagtatacaaacataacattactgcacagaaaaaaaaagtgaaatgtgatatagttcttctaggatttcataggtttttattgtctgtcccaaatctgtctattaagtaggcagacatcaagtaaatttaattgcaaatggattttagtttggtgtcagctcttaataaatatgaaaattatatttattgtattataggaaaaaaatctctttaataataataatttttaaagaaaagctttgctttttgagatttcctctctagtgaggtgttcataaagtcaatgcaggcaacaagggaaaaagaaaggaaaattgtactgctgccaaatttaaattttaaattgacttgactcatccattaattaacccaacttccagcataattatttgtttctaaccttaagtctgacagatagaattctttcaagtatgaggatgtatcatagaaaacattggtgtttatggacattggaatggaatttctttcgcccaagattcacagaagttcatggcacagggtacataataatgcaaattgctttagcaccttcttcactgggccttctacaggactgaaaggtcagggtattagaccactttttcattactaacatcacatagactgcaaaggtggcactaaggcaaaatcccagcaaagtggtgaatgatccttgctcgagggccaatttagaaaaaaataaaaatttactgggtggcttcttttcctattcaaaatgaaaacaattgtattgcatcccatgaggaaaatatatattcttcaaattagatgtgttattttagtgaatacaaggtcaatgagaaacttagcatttcttctcagctactaaaatattaatatctgggcatatttgaagagtagcaatgtggagtccacaaatatcaaaactggaagttatggtctgcattttaatttgttaaagaggcatctctagcatttgaagtaatgctatatatcttcttgataactaatattttacccttagtattattcaggagcccagtgcattcattaaagaggagaccatagcagagatgacatattttccaaccaggaggtaatagttctgccatgaaaactccagccttctgactccacatctagtgctctccctaaaggaactgcaaagttgcagggagccattagcaaatgcagaaaaatatgtgcattaatgtacttatagatgacctcaaagacagttaaacacatgaataagagtggctctgcattacattgtataagaggcagaacttgtctctcatgtcccctccccttctgctagtttgtcccgcacctaccattcatatagtattctcatttattctagaagccagccttcctaaggaaaagcctcactgtttgagtctttccacaatatctctctaaactccctttgtccctacttcaaaattaattacttttattttgccctattcctacaacgcctgctttctgcaaagtatgacagtgaagtaaacacgggctaccttcaggaaaaaaaaaaaaattggctgggtgtggtggcccactcctgtaatcccagtattttgggaggctgaggcaggtggatcacctgaggtcaggaattcaaggccagcctggccaacatagcgaaaccccgtctctactaaaaatacaaaaaattagctgagggtagtggtgtgcgcctgtaatcccagctacttgggaggctgaggcaggagaatcgcttgaacccgggaggcagaggttgcagtgagccgagatagtgccattgcactccatcctgggcaacaagagcaaaactccatttttttttttttaaaagtgtagtatcaatgttaatgatgtgttctttgatcaaatgtgtttgagtctttattcccaaccttgaagaagctttatactaaacttaagcatgttaaaagcttaatatccactgcctctaaggtagccagaccagtgtcatctatcacttggaccactccaacaaccttccatctgacctgcccaccagcactctgtccccccccacacattcaccacttggttcccaaagtgctctctttctggtagattctcggaaattaaatgatagtgttggcactgattggaatataatctaaacagagacaagtgtgaaactttgatcaaacccagattcccagaaatgtgtaatttctactttctaaaaaaaaagtaggttgtgaaaactttcaagaaaactgggatatgaaatagacattgaaatatttcaataataacaataatggtaataatagctaacacatagtacttactatgagccagtaattttctatgccttttatcgattttaaggtatttaaatttcgcagccaccctctaaggtaggtactatttcacactcttactcataaggaaactgaagaacagagaggtaagtatcctactgaagaacacacagctaatatgatggattcggaaatgaaccaaggaggtctagctccagtatgtaagtaaatcagaaaagggaagtgtgctcgttataaatgcatgacagccaaagctctgtcgttaggatataaaatgtctttcagttttgcattttgtgtgcctttgagcagcagatccatgggtagaaagtaaagagaattaaatagcgatttttcttatttattctaaatattaacctctacagctgtcatgggatgtactcaggcttatagtattccactctgcattgtgatttacacaaaagggaaattgtgctttgccaggttgacatctcattcataaaaattagagtcaaatgcaaaaacctaatctgctttactcttttaaaggtgtgatttctactaacaaaatgcaaaatacaactcttagcaaaacatggctctaaatagaatttcaaaaccatacattgattacttacatgaaaattagtcagaaacccatggtatgtatggttttaatgatgtggagagaaaaagagtggtctaaaaatggcaagtaaaaaatacattgaattttaattaggctaaaaatagtcaagtttcctattcactcacttggaaatgataaactttccaagatctattttggagcatggcaactcttttccatgtgttctaaatggtctcatttccctatatgatgactaccttggcactctgtgtacttgtgcaacgggagggatttgttctgatactgaaaggaaactttgtgtgtacttccatgttcaggctacacagccttttgcaagtgacttttatttactatttctgatatttttaaatttatttaccatatttgaggttattttcttttctaatgtaattttagctcttttatttaccgtttttaaatttttcatacgttctatatttgaacatgctttagtccaaagctttttccctttcattgctccagctcattttaaaaataataaaagaaagaatattgatctcaaaccttaactgcttacattagattatccaatttccccatgaattagaaaattgttaggatattctttattgaagagccaagattatagtaatgcaggctgattttatctatttgttttactccatcactgtccagaatcttccagctggccagattttgccccacattaaataaaagaaagggttgggttccagatataatcacaatgatgactagtgggcaacaggtgtgggagataaatgcaccatgggtgattccttgagttgtccaggacacctgaaaatataagcttatgcaggaaagaaagcaagtatgtctggtcttaccatcatactcatcttactcatatccaagctggaggtgttttattaattataatgtcttcattgttttttgttgttgttgttgttgttgttgttgttcctttttataatacttgttaagtagagctcctgaatagatattgctcagtctttaagtgaaaatcacgcatatgttttgcttttaacactattatcaggcttgttgatacatgccaagctatgtacaaacatgcattacctcatttaggctccacagcaactctgaaaactaggtactttatctctccaatttacagatacgaagactgaaaatcaaagaagctaaataaattaataatcataagaaataagaataaaaataattactattattactaataatagtgttaagttaaatttaaaactaacatttattgaaggcttaatataaagattcttggatgaatcattccatttaattctcataataatcatattattatgagacttacatgatttattgttatacccattttaactccaggaaatgatgaggcttagagacatagagatcgtataaccagaagcagaggcaggatttaaacctaaaatacctaactacagagctcacggtctgaatcaccgtcctgtactcctactgaaggcagagcacacatttttacccagggcctgtttgctgaacagcctgtgatttttcagggtctcacacagcttttaatctaaaacagtgtttctcaaatctggtctaatgatccatgctgatccacgacagggtttcagggtttcactgtctggcagcaaaataaaaagatagaagataattttaagagtttttcttaccattaagtatgttcaatttaaagaatcacactttgttctgggattatatcctttcccttctggatcaaaaatatccctcctttttatctaatgatggtgatagttgatgatagtatttttttaaactcttttaattaacaattataaatttcttaaccctatgtcccctgcctcacactggaaaaggaattgtatttatttatctggaaaattttaaaatctgggctcttttgaaaaagcaaaaatgctattagggaatgaagggtggtacccaaagcaacttctgtaagagtgtccaagattaagccttatgctaggcagtagcattttatggtttttaaagaattttcaaggatgcaggctacacttgaaaaaaaatgatgttattagtgtgtgaatatatttttatagaatagcactgttcagatacaaaacacttaaactttgactgagtcatgtgcatgcaacctactaagtgcttacacattcagcatctcatttgatctgcacccaacactccgtgacacagtgctactgtaacagaaaggaaatacaacaggatatacttcttatttgtattattcagcctctcttagcctcagtttccttttaggaaggaattgtttctatcttaaaggattatttcgtggattgtaaataatgtaggaaaaaatttggtcatggcacaaggcatatagcagtcaataactgctatctattgttgtaatgattatgatcctcattttacaattgagaaaactacaactcagattaaagcatgagttaaagtagttcatcgggttaagtgatgaagttggagcttgagacaaattctatctgcatcccccaagcttcttttggttatccctcacccacaattctatatgcatcccaaactttatcttcactatcccacaccacaattttagtaatggttagaagctgagaaatgccactgatttaaatgtgcctccaccattctgtagatatgtgtctttgtccacatggtcacagcaaataaacttttcagtagaaaaatcaaataaaatttaaaatgattcattgtacatatcaagccaataaaagttgtattagaacaaatagtataaattaaaattgtaaagattttcagatgatctgaaaagtaggatcctacagtgcataaaggaggccgtggctgaaggtcaccagtttaaggggaaggtaaaatttgaaggtatctgtaaaagatacctcattgtataataggtgcataccagcagatatatttttcaaaattatagataccaatagtcagtgtgcttatttatgtttattttgtttttggtttaaattacctagagagtgacgtcatctctctagggtagagcattgttttaattgtcaatggaatattagaaaaagtgggtgtgtcactgattccttagtaaaaagctttattttcatcataattctaaagaataaagagtttctgtctttttaaagagaaagcagaagtatgacatagttcagtcctcaaagggagaaaatgaggaaggaaaaacagtttcttttcctttctttcagagggctttgtttttctcttactgtaacatactcattagggtcccatctcttgttttgagctcatcagagagttggattttaaatagctgtttctaatatattcagagacaagtaaatactgaatatacactgtgttgtgagaatgagtatcacaaacttaaaagatttaacagttattaagtttcatatcttcctgtgatttagggtggaaatttgcagcagagagattattacatgttatgtaagccaccagacagaaaggacaacaaagtcccaatctccaaagggaagttgagttataagagacaggaataccagtgagaagtatgagagagtgggtgggagataatgtttagaatctcttttgctgcccgcaatttatgaaaatggcttgaaaatatttatggtcaaagaccgaaatatttcttcaaagaagattagctttgctccattaaaagtaatgagtagaaatattaaaaaaaaaaaaagttttaagtacctgagtatcttgccagcaactgaccaccactgctaaaagtgaggagagacacagctttcatcattgggactgcagtttatttcaggtaatggaaatattttggatgaaataatagattgactaatgacacaaataaatcattctgctaagccacaaaagggcaaaggtcttggtacataacccaggagggtgtgaaagtcactttttaataggcaagctgcaaaaaaccagattggcctggttctgctgaacagcaatcatggaaactgatgtgttgtgtcttagggactgaggggagggggtggaaaagttttggctcctcagtatctaattaagcaggactgtgctttggacagcctggtttgtgtgtgtgaatctgggtaagaaagaggtgtggtagaagcttgcctaatgtgcctactaatttttaaagttacaatttgctgccagtcttaccatattttctcaaataatttttcaaacttcccttttatcctcatgacatggaagttgatgacagggacaagaattttttttgttgttgttatttgacagatttttaaacctcccatgtcatcccaacacactgctctccttgctgcactcaagtggtgattttctcactttaatcagcaattgaatcacccaaagggcttgtcaaaacagattgctggccccactcccagagtttctgattcagtctaaggtgaggcccaaggatttgcatttctagtaagttctcaggtcattttgttgctgttggtcaagctatcaagctcgcattccttccccgggatctcatttgacatactctcttccccatcgtttccacagttcaagaggttttgagtgaagcatttcagattctttgcttcatcactacccatgtttctaatattttcttttcttccagtaatttctttccaatattccaaatttatatgagaaagaactgtgtaaataagaaaaaaaaggtttttttattccagttaaatgatgataaatataataagagtctttatttaaaagactatcaagaacagcaagacatgtgaaacctacctttttcctctcaatatctctactgattctttgagaaatacagagaaacaatgaatctctatttacttcaaatggcattcttctaactcactaaaggtgaaagttccaggaaatattttttctttctaaaacaaatagcagagtaaataatgtcattttaagtagtataaataaataaacattatcaaaccatggtatacaaagaaataaagattctaccaatataactactttgaaagctactgctactggaagaataattttataaagcacaagaattgttttatttctttctaaaaattggatacaaggcagtaggtagaaggatatttatatttttgttttatattgttttctgtattcatgattaatttcatcagtaagaatttttaagaaacagtaaaaaaaaaaaaaaggtgttaaaaaagactacaagatttaagtttgtttattcaactcacctatattgagtgcctagtctgagctaagtattattttaggcaattataataaagacaaggcgacatttccctgaaagcctctaaatcacaaactaggtaataaacaaataaacaaatataactaaaagattaaaaagagttagggtcttgctttttcaagggtcaaaacataccatgaagctgaaatagctaaagtactacagtactgacacagaaaaggataaatagattaaggaaacaaaatggagtctaggaacatattggaatttagtatatgataacattggcattttaaatcagtggcaaaatgacagaaaattcaaagcagatatctaccttctatcatactcagaaacaaatattgatacactaggagaaacacaagatgatacatttgttataatctaggtgttataatcataaacataacacaaaacccaaaagccaaaagataaagagagaatgagacttgactatatctaaatataaaatttctatatggtgataatttctgaaaataataatatgtcaactaacagcatggaaaaagtattcacaatcaactaacagcatggaaaaagtattcacgatgtatagaacaaataatattcattgaacacatattctgtaccaagcaccatactaaaaatttacaatcaccatgttatttttaagacttaaaaactatcatttttttcttgtatatgttaattagcttgatttagccattccacaatgtatacatatatcaaaacataaagttgcataccataaatatatatataatattaatttgtcaatttttaaattttttaaagaaataaaaactatcagatgtgtactcttattatcttcattttatacataagcagtctgagattggagacatttggtaaagtgccagtcatctagcaagaaagaggcaaaactgggattcaaattcagatgtctctgactccaaagttattaaacactgaggtaaaaagagtttctataaattcaaaataaaaaggacaaaatgtaggaagtcatgggagatgaaccaaatggccaaggtagttatgataagacatttagcctcactaataatcaaagagaagcagaaaatttagtattacttgtgaaaattacaaatcccatatgctttggctcagcatttctacatattttctaccctacagaaatgcgagtctttctgtacatatctttaggtgcatctagaatattctttgcaatatcatttgtagtatttcaaagacggatattgcctaaatgccaatcaatagggaaatactcaaatgtattatgccatacacctatttcaaaagaatgaggcagatctatatgtactgacatgaaaatatggtcatcatatattaagtaaaaagaaagtttcaggaaaaaatatatatatatagataaaaaagatcctgttgtgtaaaattatacattctacacatgcctatgtgcacagaaaaatgtctagaatggtaaacctgaaattattaactgtttctacttctgagaagttatgttggagaagtagaatgggtatgagggaaaacttttacacataaaataaattacaaaacaaaagttatctccagtttagatttcataatttaaaatatgtattttgttaaaggcaatcagaaaaaaatataatggaaaatattttagagcaaattttaatgaaaaagtgaaagacatttaaagacatttgaactcaaacagtgtagaccagttgttcttgaagaggatctcagaaatccaggaaaatacttctcacttatatctgactgaagaatatcagtgccctttagggtcaggctttgctttcatgccaatatcaggccactgataataaggtctatccaaattccatttctgttttcatcctccatccctactctactgttttaaagcaaacagttaccctggagaaaaaaattcaagtatccccaagtatcttgatgacttaaggataacctcaatggattcagaaataattccatggccatgttcatttaggaactctttgcaggatgcagtatttggttaagggaagccctacagtgtgaaatatctgtcccaagcaattgctatttgagtcacctctgtattacagaagagttttactcttttcattattgttctaaaagatctcaatggacatgactgttttcattaaaataaaaatagggcagaaaaacttgttcattcataaatgcgataaatatctgtccactgatcagcccaagtctagtttccttcctatcaagttcttgtttattaagacctgtttttaaaaatatgatttcagattatatgttagctttatgcttctacccgttttaataaacatatgataaactcagtgctgtgcttaatctatgccactttgatataattactggactgtaccaataaactatttgccaagtttggaattcaatgtttcccatttgataaatggttgaaatggtaaatcctttcaaaacaactggaaggtagacatcattcaaaatgcttagggtttttgttgttgttgttgttgtttccttctccataaatgactttttgcagtttaaaccactatcaaatttatttggactgctaaccctagtcctttcctgtctattccagatgaccccaggagtatgcaggcttgttatttcttccccatagctagtttgagagtaatgagtcagcctttttctagcacatccatagtgcctcaggaccctgcattaactgtatccacactgttttcaagcacagaaatgatggggcattgctggagcatgatcagataaaagtccccttccaactatcaaaccctgacctccttttattcatccgaggagttctccagtgctctgcagagtactgacacatgagtagtatgcctgtttgacatcccctctcatctggctgggacctgcttacacagaagttcccagcagccctcaagcccaagggagatggaacctagcaattccctgtaaccagcatcgcctgtcccctcagtggggtcagtacccagactctggagtcagagattctaagatcaagtcttggtcttgctagttactgccaatatggcctacaaaaaacaatttaattttctcatgcttcagttttctcacataatcaatagggatgatcctagctctcagatttactgtgtcaataaagactgaatgattaaggttctcagcacatgactagtgcagaataagtgctcaaatagtgatattgtgtactttctaaaatgacatgtacgtataatatatatatatatatatatatacatggcctggcaatttgaacatctatcatctttgctttcagaattctgcacttatcctaacctgggaaatgggtcttccctttggaacgtaatgcttagattaaactggaaataagcctgctaatgggtcttaaactttctgctcttcccaatgtgctattgctggactctgtattgctatagtcctttctcatccctgcctcccgtaggtttagctaccttgttctcatgaccagaaagggtaaaagatcagtataaatcccttgttggaaatgaattatgacagggttaactttaatcattgaggtcacttgtactgtctagcttctatcttctttgatctctttcctttctttaaggaaaatttgcatatgtaaggtggttcttattctataagaaatagaaacagatatattctagcaagtgttgaaaaccttacttgccaaactctaggaaatagaatttcatgagaaataaatatattgtctaacacatgacttgtaatagaaatgatgtttccaacaaagcatattttatacactaattttcatgatggcttattaaaaatgtacaacttctcatataaaaaataaacagataaaagcagtcttgtggtaatttgacaaatccacgtgaaaatgtcacactaaagcaaaaagtggagtacatatagcaatggaaggtttaatctctgaccctgacaagttttacagttaaagtttaaatccagatttcttaaacaaactaacaaaagcagaaatgacaaataggtttcttttcatttgtctgctctagatctctgctttgagaaggatgctgaggtcacatctaggtgcagtgggatttagtgacgtgttagaaggcaatatctatcatggcaatacagtgggatagcatggatttgtaccatgtagttactatccctgcttgcttttggatcacacagacatccttttgtatagtcaaatatgatataattttgtggagaagaagacactaacatttggtggaagagtacagaagattaatctgaaccaagtgcttttcaaagcacttcgcatgtactcactcatagaatcatcataacaacactataaatatatgctgttattagccccattttccaaatggggacattgaggcacagaaaccgtcagtaacttgcccaagtgatacagctaataatgagcagagcttattgacagatctgtcaggaagtctgaccccactctgtgtctttaccatttgtattaatctgcctccaaaatttgttgtgcaaaaaaaaaagggtaagttttcattaacatctttctatggcatgcattttgtaaacctagcaccacagaacgttcatgtaacaagtgatcttgctcattatctggaccaaacttctgtctgttaaaggatcctgtccttaatacccagtacctcctcaggatctcccagctcttcccttgttgagcagctctggaaactctttgacatattgaaccaaaatctccctacctctgatttgcacctttggtggagcaacacaaaccaatctacactgtcttccacagaatgacaatcaacatcttgtcagtgaaatatctccaccatgggtgaaattcgatgagaaaagagatagcagatttttaaaataacccataattggataaaagtcattttatttgaaaactttttccctctaaaataacgtgatatattttgaaagaagaaggtgtgaggtcaggtgtccattatgtagccagacagatggatacgttgttgttttaccattgccatagaaacaagtgctatgtttatctcattcagtgaggaagaatttctcgggtctttcccaaaagttgtgatatgtgctacactgaatatggcccaccatgtctatctggacaaaataaatcccctttttaccggggaggagtgctacacagggaagaggggaactctacttgagcatttcttcctatgagctctgtcaatttcttgttatttgagattcacaaattctctttaatataacaatctattaatgtgctatattcatgtttggagataatgctaattgtcaaagaaattaggcttctagttctaacctctagagaacatgggaaaagaccaaagaggagtaattacagagatgtggtttaatataaaggttttatatcaaaccggattcagccatactctaggccatgaaagtggcttttgtaaagacggtgctgaataaaatatagcctatcaatgggttcacaaagatcagtatttaaccttagttttagttgaaatcatatttgtaggcccattcatttgcataccaataaagctgaatcttagctttaaatgacttaatgtaagacctgaaacagtgtggccttggaaagggcatcacataaactattaggacacccgagttccatctccagctctgccaacaggttttgtgactttggtaagctcataatatttactgagcaactgtttggttttgagaagttgagctgggcattttaccttatttaattttcatagccactttctaagttaggtaatacatttaccttgtacagatgagaaaatttagtctgaagggctaatgaacaatgcccaagatcactcagctgataaatggcaaggctagaatgtgatgactcaagcttttctctgcattaaactattcctatcacttcacctcctgagttttagtgtgtatctttgtagccacgaggtcgacatgctctgcacattctttgccacattgacattctttgatttcagggtaaatatttcaatataatgtcctgttctttttattatgattatccatttcactgactgctgacagacctttttcttaatttgtgtcatctatattgaaaatatatctcaatcatatttaccaaaatatgaaatctctacttaaatgggcttaacacactgtatcagcaggatataatttcacatttgtgttatctgtaaaataaagcaaaatactgcagaatttgactcctacagtccaaaactacagatacctttgctgacagtagaggtcactggcaccttccatcctctccatccaggagatgctccccacaaagggtggttcttaaatttgtttcccaaagggtcaggggatggtagaccagaagctcctacaagattttaccaaattcctctttacttttcctgtcacttttgacctggtgcctacaaataaagttactatcgtatttgtggaaatggatcccaaaataactggcactaaaaaaacctgtaaagaagttgttgttatggttgtttgaacatttaagaattgattatgagcatgtgaatcatgtaatcaaaaagtcctcggtaataaatagggacatcgcttgtcaaatgagttggaaaagttttgcctgcatatatccagagcttcatccctgacaaaggctcttagaaccctccctgatgaaatacacgttttaagttcttgagcgcatttcttaggagttgacaagttctccataaggtggcgcagtgttaacactctgttaatggctttttgccttttttaaaaagaattttcttcaacttgtttgaaaaatctcaggatggacttgcttttatacatagtgttttcacatacactcagcatatttactccatttgtagaaatatattttgaaattaagtaattttcaagcttttatatcttgttttgtgttggtttgctaattttagactcactcataatggcttcttccttcaaatatttaaattatacatgtctaatcattttttcttcctcagagataagtagaagcaggtataaattaaaaacttacctttaaatattacagatgtgttttgtaaacttcttggaagtgaaaatttgcagaatcaagattattggtgttggattgctaggagctggtatcacaggtgcactctttgcagctgaaccatagtagtagctaagtaaacaacaagaaggtcacatgtgcaaattatgcagatgattcagacaaaaacatcaatgcattttagttatttgaatatatcaagtaggtgttttgccatcgcattcagatattgttcttgtctgataccctttagcaattgttacaaaactctgtctctcgtgtgtgtgtgtgtgtgtgtgtgtgtgtgtgtgtgtgtgtgtttagcccaaatggaaatgcttggttataattattgtgaattctaaaaatactgaagtggccaacattgaccaaattgacaagacttttttttttttttttttttttttttgagacggagttttactcttgttgcccaggctggagtgcaatggcatgatctcggctcactgcaatctccgcctcccgggttcaagtgattctgcctcagcttcctgagtagctgggattataggcatgcaccaccaggcccggctaattttttgtatttttagcagagacgggatttcaccatgttggccaggctgctctcgaactcctgacctcaggtgatttacctgcctcagcctcccaacttgctgggattacaggcgtgagccaccacacctggcctaaattgacaattcttttcactaatttttctttaaagggaaaacaaaaccatatacatagaatatgtagtaaatgctgtctaaccaagtcattatttttcaaaataaagctgattaaatattctattctaaataaaaaaagattccacctcacattttgatatattgttgtatttagctaaagtgcaacaaatgatgaactctatcatttatgttctgaagcaaattattgcatttaattatattgccagtctgatctcttcagaagatgagatttttagtgattgaaactgaattgcctatttgtgctgtttaaagcactgctctatcagcagcatgtggtcattttggataggttagctcctggatagcatctcttgaatgtacttatcatggtaatatttaaacactgttcatggtgtttacatctcggttaggttataaagatacatagctgcattggctttgcattttccattaactttatttaataaggtcttcgtcaagccctaaaccctaatagaggaggtttatctggtcagattgtctgaatgaattttaaaatcaaagtctcaattttttgctttgatgttttcatttttgataccttaaccacaaatcctaacctaaaattattctcctagaaaactttctatggaaaataatgattaacttcatctcttagtctccagggtaattgtctttatgttatttaacagcaacttcctaagctgagtattttaaatggtttacaaattcagaatgctcaggaaactggaaaattcagtagttgagaggacctcattctcccttttctatcagatatcccttttttctctctaccttccctagttgagaaaagggaatagactttagaactaagatagtcatttgaaaaatatgttattgcccttacttacaaaaaaaaattggtctaagaatactttctcatcattcagattttaagattccatctgaataaagagttcagcagctaaaataataaatggtgattatccaacccctccactgtaaacatgagaacatgaagtcacaaaaagctaaatgatcaactagacatccttcattgaataattggcagaaatgtaacaattagaaattctgtatcttttttgtcatgtgctttaactatatttccatttcccttcccaaaaataaggaaataacttaaatccaacgtaatacctctttacttgagaatttttataatcacagcattacaattctcagggggaaaaaatggtataaaaatgtaaatcttaataaaagtcccagggagtaaaacttgaaattcatacacacacacagacacacacacacacacacacacacacacacacacacacacttcttctggattgaattattgcaattattatcaactcacaattggccaagacaacataactacattatgaattctaataattcctaagtagttaaagtaaagcttttttttttccaaatgcttctcttaatgagatgaatgaagtattttgttttgggttttttttttctgtttttctttattttaactagttcatgtatccatgctcaaagattctattttgcaagaagtacacaactatcaccctcacttctattcataattttgttttttctgacctcattgctgaaaaaacatgtttatatttaaaagtaggtgaaattggagttttattatagttatttccctcggttctataaattcctccctagttatatgtcaaaaatcacatgatgattgatcattaagaattatgtgtaatttttcagcttaacaacttatttagaccctcctacaatgttgttgaaagcaaagaactggaaaccaccagacttgcacaaagctttgttaactggtcattagagtcattcgctttgccaacaagtcctagtgtttggagccaagaggttctttatatccccaaaagatgctctatccaagatgaatgagaggtgggctattgttttgtttgttcttttgtaaagtgggagaagggtgtttatgaacaggagttagagaaaagagagctccgcacacctcagacaagttgtaagacagatcagtcttagaaaagagaatatacagagattaagaactcgataattgacactcaaaactacttatatgcccatcccttagaaagtatttccatagacagggttatgtgtacacccacttgaaaaccactgtcttagggaagtctcagggaagcaatgttctgaagcatgaggaatgtggccttgaaataaacatctgttctcgtgcttaagcaatcagcacagtcccataagctccttttttaggggcaaactaccccccgtacaatgttctgaggcctttgatgagatggcacagctgaaagtccatgatacagaggtcaagtgaattcagaaacagcccattaagttactgggaaagtaagttactgggaccaattacttgctttgaatactcaggtgtgactttccaaatgtgaggaaacataattgcgtgggattatctattgctgtgattgagctgggagcacctgcattctaattcaaaccccacatggttgacctgtgtggacttctagtagatcacccatatctctgctttcctgtccataaaacagaggagcaaattaagtatggcctgcttcaaattctctgttgaatgagtattagtacaaatgtggttttcatggacaaagccacctacaaaaattattcacattaaatagtatgcttctaatttttaaggcattggaaattttaagtaacaaaaaagtaaatttgttagcatgataataactgacacttgcaatgcaatttcaagtacttttcattatttgtttatatatcccccaaagttttctgcattcagaaattagaaatcaataaactgcagtgctatctatagcattatgtataacataaactataaaggggactgtgatctataaattaacataatgttttttgtgaccctaactaccagctaaaatatgcactctctagctttgatccagatctaccaagagtggagttcccacaagtcagataacctgtcctaagaggtccgtaccatgagatcaagggtctttttccttgctttttggcatttgctagagtcaggtgagatctgtctccattctgcactttcttctcagtgccttctgagagcttgtcccttcatatactcacataattcctttacccgactgtgggaggataaagatgagaatgaaggcatacacagcagtttatcttgaggaagagaacccaagtctgcatccagtttctcctccaatctaataacatcctttgaattagaagtcgtgcagggtgtggtggctcaagcctgtaatcccagcactttgggaggccaaggcaggtggatcacttgagcccaggagttcaagaccagcctgggcaacatggcaaaaccacatctctaccaaaaaaaaaaaaatacaaaaattatccaagtgtggtcatgcgtgcctgttgtctcaagctactcaggaggctgaggtgggaggatcacttgagccagaaaggtcgaggctgcagtgagctgtgatcccaccactgcactccaccctgtctaaaaaaaaaaaaaagaaaagaaaaagaaaacaaaaagtcactcactcatgggcccagcttcaggacaaatgttgggaagccaccatttcaggctctgcggccagacagtctgggtttgacttccagctactgccacttaaaaactgtgtggctttaaatacattatttaacctttatgtaaatcagtttctcaatgaggatcataataatagcgtctttgtcacagttctctttaagataaagggtaatcagtcacataaaaatagtataaccatataagttgtcattcaaactggtatgcttttgtgagtgaacagggatgttattaaaattatatcaggacaacaggtgtgagctagtactttcttgggaaatacaggacataggggtttgctctataataaaacacctggcatgatacctggactttgcatgatacctggaccttgttagagttaatgggctattaacatcctattaataaaatactaatattgcctatgggattatggtaaaaaaatttcttagaatatggcattctctttcatattagcattttctgaatttgttaatttgcaacagtattttttttgttattttctttcagtaaggggagagagtactcaaatatatatgtgtaggagagcttcttaccactatttcacttgtttagcccctactttaatccctaattaggcttagttgctagagaaaactcactttgtccaacagaaacctaagcatggagtgtagtgaggagattccttgcttttgctccaatctccacccttgtcagatgatgaaaaaggggtgatatgaggtcgatcccatggtagtagcaggactaaagtttaacttcaggaagttgacaactatttttgtttagattaaaaaaaaatttaatactgtgtcaaaaatccagcaaaaataattggtaattcagaagtcccatgaagtaatctctatacttcttgccagcatagggtctttttaaaacatgttctcagggtcctggctagcctagcatcagctgccccaaaaatggggcttccatcattttttggcaaaatatcatcgaagtccagcaggcaaggttgtttgacctggatcttttaattcaacataatcctaatatgaggacataccacgttaaatttctgttaaatacaacttcttaaccagaattcactttgtttgagcctgtctatctagaggcagaaattacactggatagttgaaattcttttaatatttattgcccttctaagtctaaatttaacaaaaccagcactctacagccagaggacaggatttttaatctatgtaacatgaaaagcaacagcatatgtgaaagtcatattttattcaggagccgactaagttcctttattcctgataataatatcaactggaatagcatttgtgtaaggcctctgtgtttacaaagtaggccaagatacatatatcacatgagtaccttatccttcccatcccaccaacttaacatgcagcaggcccaaccttatccatgaagcagcctaacgaaaaagtccaagcagtagtaccatagagcatgttttctgcccttctccacagtctggggaaagcgtgagacagctccagaaaagatgagttcatattcagcctgggaaagctgcacagtggcctttcctgctgctcagcatgaaatgagccatgagcagcatgaaatcactgaccacaggtgtagagattcacagtgacataaatatctttgctgttgagcagttggaggaaagctgtgggaagatttgccataagctcctcctggccaaatctgcctttataaagaaatggtcatttaggtcacactgttgctgagaagcttggtaagtcccattccaacttccaattaaaagacctacggagaattagaaaaacatgtagatgagtctatatctcagaccatttgaaggagaaaagcaaaataccacacagtgaaaaactaaaatcttttatgtgtgaaaaaaaaccttcataaacaaaatttacaaagaaaaataaaggcaaaatcacaatctatataatagatgttattaaaaatacatctaagatgttttaaaattctttcaaaaatgacaaactaatatgaaaatatgcaaagaaaatagaaagcaaataccaaggggagcaaaaacatatgaaaaatgttcaaatttactaatgatgagagatgtaaattaatgtaattagtaccatatttgcctacaaactggaagactttttaaaaaattgtgaacacccagccttagctagtatatggagactctatccctcttgattttaattttttaacccttcttccattttattaatatttggtatcagaagaaattttgtttttcttttattcattaattttggcattattttttttagtttcttatcaatgtaagcaggatatagttttttttaacaaaattaaaagttactatctcttaacggaagagtgtattccaactacatttatggtcataattgtcatgattggtcgtaagttatgctgcaaatttttcaatttctattctgcttttttattttgtttgtatgttaagcagtttatgtcttgtttacttttaatttctactgaaatataaaaggcagaagtactgtagcaattattatcacatacaaatataccctaagtactaattgtaataattactgaggccagtaacagaagaaaaaatacaattaaacaataatttaaaatattaaatatgatagctttaacaatttgatagactaatgtgtgttataattttggtttaaaaaatattggacctgaaaaaaaactcattggaaaaaaatcttagccaaaaaaactagttacagtatgatcccaaatgcacaataactgtacattcttaggtgtgtgtgtgtgtgtatatatatatataatcatgggccatacaatgacatttaagtcaaatatggactgcatatacaacagtggtcccataagattataatggagctgaagaattcttatcatctagtgacgccattgccatgctcacatcgtagccttactttccatgcttgtggtgatgctggtgtaaatgaacctactgcactgccagttgtataaaagtgtaacatatacaggccacgcacggtgactcatgcctgtaatcccagcactttgggaggctgaggtgggcagatcacttaagatcaggagttcgagaccagcctgaccagcacggtgaaacccggtctctactaaaagtacaaaaattagccagttgccatggtgggcacctgtaatctcagctgctctggaggctgaggcatgagaatcacttgaacctgagaggtggaggttgcactgaacagagatcgtgcgactgcacaccagcctggtatgtataactatagtcctatatacaactataactatagccccagagcaagaccctatctcaaaacaaaaaacaaaaaccaaacaaaaaatgtggcatatacaattatgtgcattacataatatgtaacaataaatgaatatgttactggtttatgtattactatactataccttttattattattttagaatgtactccttctacttacttaaaaaaaaaaaatcagtgaactgcaaaaccatctcaggcaggtccttcaggaagtattccagagacaacattgttatcattggagatgacaactccatgtgttttattgcccctgacgactttccagtggaataagatatgaaggtagaaaacagtgtattgatgatcctgaccctgtgggcttaggctaatgtgtgggtttgtgtcttagtttttaacaaaaaagtttaaaaagaaaaaaattaatttaaaaaatcttataaaagaaataaaatatttttgtacaaatgtacaatttgtgttttaagctgttttgttaaaaaagagccaaaaagttcaaaagatttaaaagctcataaattttaaaagttacagtaagctaaatataaattattggagaaagaaaaatttttttcataagttttgtgtaggctaagtgtacagtgtttataaagtctacatagtgtacagtaatgtcctaggccttcacattcactcaccactcactcattgactcacccagagcaacttccagtcctgtaagctccatttataagtgccctacacaggtataccattttaaaaatcttttaaactatatttctactgtatcttttctatgctgtgatatgtttagatacacaaacacttgccatcatgttacagttgtctacagtacccattacagtaacatgctgtataggtttgtagcctaggagcaatagggtatcccatccaaccaaggtgtgtagtaagctatatcacctaggtttgtgtaagtacactgtgtgatatttgcacaatgacaaaatcgcccaaggatgcgtttctcagaacttatctttgttgttaagcaacatatcactgtatatatttgtattcatagaaaaacagactgagaggtaaatatatcaaaatggcagcaaagattgtcacttatttattagtttttggtgactttttctctatgtatttttttctaattttccaaattttatgtaatcagccatactgtgtttatatccaaaaaaaaccagctactaatatgaattttaacataatgcttagcaattagtttctcagaaagtaaaaacatcacagatttgtctaatgccctatagcagactgtagggattatttagaagtctggagatccagtttaccgtggatagccacagaccaaactctttaagaagggtgaatagttccagccacatagctcttgaaatttgcacatggacaaatggataggatggaacttgaattgcactcagcaaaactttctttgtacgttagcacataagtcacaatcttgtggtgtcctctagcccatagaatcatataccaagagaacataaaacacctcagcagctgatttttttaataataaaagtattcagtatatttttctgaagtcaataggaatttaatgcaaatatagaagtgaatgtatcttctcccagtttctagcacttaacttcaattatgttgctcttttaccataaagataaaggccgggtgcggtggtcactcctgtaatcccagcactttgggaggccgaggcaggcggatcaccaggtcaagagatagagacaacatagtgaaaccgcatctctaatgaaaatacaaaaaaatagccgggagtgatggtgggtgcctgtaatcccagctactcgggaggttgaggtgggagaatcgcttgaacttgggaggcagaggttgcagtgagccgaaatcgtgccactgcactccagcctggcgacacagtgagactccatctcaaacaataataataataagtttagtgtttcatttacaaagtcataatttgactttgatgctttgggatttataatccaatttgtatgtcttgttcagcccagtggagtgcatttgcgtggcaatcatattttacaaattataaaccaaaacatatcagtattttttcagtctttgtaaggctatagtgagttctcatgccaaatcactcatttgtcatttttcttaatgaaatttaattttttgctattaatattgaactattttctactgaatcctgattgattatgagtgggcaaagagagatgctgaattaagttgatttacagagaaatgttaatgtttatatgctcccattctctgtttctaccacttaaatataataaagatgtataactttttatacatataaaatataatatataaatataatattatataatataaatataatattatataatataaatatataatatatataaaatataatatataaaatataataaagatgtataacttagagctgtcataaagtttgagcatattttaaaattaggacagttagagataaaacaaatacaaattatttaaaaggaataagaaaagatagagtttattagccatttaaaatatactgattgccattgggcactaattttgtctccaattttgctgtaagtaaatgcaaataaagaaacacagggagttacataaagatcaaaatagttggaaattgtataaataaatgtaaacattttattggatctcaattcaaacaaaattttattacatggtaccatatatttaaaatgttgagcatcagcacaaaatagtttcagctacaattttaccaaaaacagagtgactgtattcaaatgggccaatgtatttacctgccatacatacggaaagagtagtatcctacccattgactgctgtcttcctacttaatatacacatgcaactgtttcacaggcactgaaaactcaccaggtccaatattgaattacctgcttttcaagtcctgctgaattaatcggagtttttgattgcacataaaaattaattcgcagtcatcggggcaacaagtgttctgttggaagaatcctagggcagcgaacagaatctgaaagcagctactctctttcttctccccacaattcccccaagggaatgggactaggcattaaaagcctagtgggaaaccaggaagtactttcttattctcatctctgctgctgtatgctcctctgcttcatttcactgcagacctggagtatcttcaacagtggacgggggccccacaaatccctactttgatgtgggttctccagttcaggtcatacaaaggcgtgaactgcttctttcttcttactctctagaattgcatcagagggaagcgtctttaacttcttggggcacaccatgctctttccatcacagcctttgccatagaatgctctgcctttccccattcagcaaccaccttttacatatggaaaaaccaagcctttcagaggcgttaggtaacttgcacaagaacacacaataaatatcagagctgggatataaccctaggtcatacatgcctgattccagggacctataccatctagaaacgactctttgggtgtaattgaccaaagaatacattgtcagagactcataaggagagtcactgatttcagaggaacacttccttgtagagccatgcattcatcaaccaacaatgatttacgtttctcagtattactagaacactgggctgagcaactggtaactaaacagatatataatactagatcctaaaagcccctgattgaaatggcagaaatgagataatcatcatacacctactctaaatggcaaccaagattttgaacacataaaacatgatatatttgccaaacaagtgatatagtcaataaatgtagatcatcatatgctacaaaattcacaagaagcctcagaggagagatgggcctttgatttaagtgattgggatttcataaatccttaccccagggattttaatggactttatatataaaaagaaggagagtgagaataaaacttcctggggagctattgtaacatatcaacatattataatgtcatataccgaataaagcatataagatctatgagtttagattcttctaagagatgaaaaatacaaaatctggaagaagaatagagtggaatttcttcatctgagactcttaaactctagaggattctgtttcagaatataataccatccccatatatcagtgtcacctagagtttgttaaaatacagattctagatcctcctcagttgtattcaactcatatctcaggggattggtctcaggaatccactgtttaaataatcatctgagcagattattttattttgacaacctttattccagagggtgcatgtataagctctaggaggtatagcacacatgaaaaggttcatgaagtcttgagcatgtacacctatctgtctagagaaaggtcagtgactttcatcagatgcacaaaaggggtctgcaatccaaaacaaaaacaaaaacaaaaacaaaaaacaggttaagtatcaccagaatataggaaagaaggccaaggtattttaaagattgagaaacagaatgatttcagtattaagggaataaaaattacctagcccaggatagaagccccaacctgagggaagatctgagcatgcagtgatattaaaaagatgataattccaaaaactaaaaataagtatttaccatctccactgaaggtggaagaagaataactatcataaaactatagcagaagaaattgagacaggaaataaatacttcttcacactaaaaatgctaggaataccccagagggtaggaataaactagataatcatctatccaggaagtcagacacacgaaaagatatgttatccaggcctatctgctttttcagcagaacacatactatgcattcttggctatttttgttattgtaataaccacaaaggtcccaataaagatagagactttatgatatttaaggtattttgagtgtgtagctttattaacaatttaagttagtctcttgttatatgttctatgttcttcattaattttccattattttagcaagatgatcactactacagtttagtgatttcttttaaaaggcacatgttgaacacattatttggctaattttctactggaaaaatatgataaatgttttatttaataatacccattttaaataaactaagtttgcatttatgaatgggataccataaaatacattgtcttgggacctgactaaaacgaccccatataaactagtcaatttcattgatttggtcaggaaaatacaaacatatcaaatatgtagtttaactttttataggaaattaaaatgttgacttttcatccagattgtttatatatatatgtatgtatgtatgtatgtgtgtgtgtgtgtgtatacacatgtataatcaaaacaaggataggaagtttgtaaaatgtgtttcatggaaagcactgtaacaagctagttaagtaattgacataaaaattatttttgttaatttttgaaatacaattgcaaaaactgattaaataagaagctaattactaagggctaaatttaaactaataataaccataaaaaactgtatattgaagagttgaaaatgtatactatagaactaaactaataaaaacatctaatgcaacgtagtgaaagaaaaataacatcgggagacatcagttcagttcccatttcagccaccaatatttgtcttaatttgttctgatatactgcagttccatggggtaacctagcaaatgataccttcattacccaaaatattgaactatattcttctcatctgaagtactaaattcacattagaagatggtggcaatgagggaatttcactataacctaaaacttggaacattgtataagagaaaaagactttattacttcactaaacaatttaataaatgttatatgaatgcttatttaaaatgaggaaaattgtctcacaactttttctttacattggtttttttttctttttgttttcatagaatttcaagctcacaaaaaagtcgcaagaatagtaacaaaaaatcctgtatgttctttatccagatttaccaatccatgttctttataatttttgatatttatgttttttctaaatcacttaatgctaagttggaaacattgtgctactttaccctaaaatattttgtgtgtattttctaagaacaagcatattcttttacgtaactacagctgagttctcaaaatcaggaaatttaacagtgatataataccattatttaatgcacaattcatacttaagttccaccactaatttcttatgtcaagaatgacccaaaagaaaaaaagccaacatgagaaaaaataaacttcatgagaaaatacaaaaaaattcagaagttactgaacatatgaaagaaacaaaaaggtagggatagcattaggagatatacctaatgctaaatgatgagttaatgggtgcagcacaccaacatggcacatgtatacatatgtaacaaacttgcacgttgtgcacatgtaccctaaacttaaagtataataatgataaaattaaaaataaaataaaaaggtttcctcttgggggaaaagaaaaagatgatgcagtccactattctctataattcttagaggttttactctttatataaaatcaaaataattcagagttttctgcaaagtctctggtttttctttccaaaattagtcaataggacacgttaacatttcttaaacagaagtgaatggcactatgagacagatttctctaagcggatcattagagcacaaacacaattaatggctaggcctagtgacaaagtatgccattcacttccatgaaatagtgtgtgtctgtctaggggcaagaggcatttagcatagaaaacagctatcaggatttatttcccactttaaaaatgctcttacacttgctgcacacacaacttaaaatcaatgactggaagcaggaaagaactggacagcttcaccagttaagttactccttttcctccaagacagtagcaaactattaacctgaatgcattatctacactagtatagacatacatacagataataacattaaataaatacataaagttattcaatgtgatacaggttaagaaggctgagatttgagtggatgcaaaacactatttcaaatatatttttaaattgaaaattctgttttaaccagagaactttacatattatagcattagctgagagagttcagttttgtttggtaattccaaaggtcccacaatatggcttagtcagcatctaacttcataatgtggtcacagaagtcatcaccatctcttaggccaaggacaagtccataaagagcatacacagcccttggtaaataagttgaggttatttatttctggttttaaatatgaaattatttcaattttgacattctgttgtaattttctttatctctcaagtgaaaactagtggaccaaggagagatgccctatggtctctatgaaaacattattcactcacgcattcaaaaactgtgagtttattcatagaagtaatagtgtcataacaatcattatctgtgtgtccattttcagttcataagactttagcatacataatgttatgtcatcctcatacttactcgtgaaaggtgtgcttaagtgctaaagaataaaaagtgaagcaagacaggcccatgtacttaagaaatttaagcttacatggagaaatacccagctaggtgtaattattttagtgggaagaataatatttgcccttcagcttgtttagaatatattatcccatagatttcagttgtgtaattacctatgtctcatataaactatgtcttattattataggaaatgctgattgtacttggagaactagagcataagtataaaacgaaaaatcgtaggtcagcaagattcaataatagtgagttttactttcatttttctgagtattcaagtgaagaattatggaaagattattaatagccatgaaacaatttgtaaataatgttgagaggaaatagagtttactgagctgggaagattagcatcacagcttaattactgactaatttaattatcttgggcaagtttttaattctctaaaccttagttttctcatctgtaagatgaaggtaagagtcagtctctctactggattctatatagattgaacactacataaaacacctagtataactcaataaatgttgtcttttgtatttgaataattccgtatgggattatctatatatttctaaacactagctctctggactaggattagaataaatatttcttgtaacagctgtgtgtttcttcagtaaagccttgtgctgcaatgatttgactggaaaatgaaggaggaagagaaaaatgtaatgaaactccagtttcagaagaatatttgttgtcttcagttctataatcactgttaggtatgagtttaatactccctcattatcactattagaaataaaaacgcttcctaagaatggcctcttttgatattgtaattagtaaaagtggaattttgttggaaagggtactgacatagttgagataatgtctacttacatttatatctttattcaaaagcatgacaattatttttgaaggagtcaggcttaccatcttgagcaattctatgcactcgtgaccagagagactatatcttaagaggcagcattgtgtacagggaaatgaatgagctctggagcaggctctcctactttacaaatctcagctcatccatttactttctgtgtgattttggacttgctccttaatcatactcagctttactttctactttttaaactggagataataatacatatcttgtattatatgtataagtacaatacatacttcttagggctattgtaaatattagactgttatgtaatcgttgtcaaacacttgatacacggatagtaaatcatgtttcttagctttatatttagctatttggatacagaactttatgacattgacaaaacctttatagtcatgtttctactttagatcattctttttatagtggtattttcaaaattatacttctgttagataaaggcattgatcctttattaaccaatggtggtctgttttctactataaaatgtgggataaagagatggtctctagagcaataagagtttctatacaactgctaaacattatgaaggtaaaagcaggctgctaacaaacccatgagcagtgacctccagatttacttcccaaagaaataacatttatagcagaagaaaactgagtacttttttaatttactgggaaaggagaaaaaaaagcttctaaaacattgacactttttaggttttagatctacttacctactaactttcatgagctaagaaccccatctgtatacttcctttgaatcagagtaaatccagcctttcagagtgcttccccacctaaaccttccagtacctgtctaagcatctaacaatgccacattttttttaaaaagtctttccttcaacctcctctcccattggaaccttatctcaatgcttcattaacacagaaaccaatagcagtttagtgcttcatccctcctattggtctttgcacttcaacagaggtcaaaggctgataattacattgaaagtatgaagcgtaatttgtaaaaagtgtcaggcttctggaggaaagagttctagaactttttgcaaaaataaaatcatactggatgattggcgccaagccatttttggggcattttagccaatacagaaaaaatgaaagtgtctatcagacatgcttttggcttattcaaaataaaaaccacattaattgagtcccatgaaaaagggagtacagttagtcctcgcctaatattgtcgacatgttcttggtaactgtgactttaagcaaaatgatgtataacgaaaccattattacatatttcaaatgtaataataaaatgaagttgaacaaaactatgttattagagtatttttcaatttagattaccaatttcattgaataacaaggataaaaatgagaaaattggcatcacctttatcctctaaaatgttagtcttcaggttatttttagagaattaccatctttaatccctcaaatgtgtttctaggtattttcccactttatacttgtcacaatcctggaaggcaagtactattatcaccctcattttgtgaaagctcatgaaagaaaagtagttagggttgagtaagtatttttttttttttttttttttttttgctttggacatgaggaatcaaggctattatatgaggtagatttgtattgaattcctgcagaagtcctcttgggtcttttggcaaaggatcacccctgccatttgtattgtaaagaattgcttccataggccacaatctgtccactcttccgaaacctaaccaggtgggctgttagtaagtctcttctaatttagggaaccattccagtgtctgatataaagtgctttaataattttgtttcatgtatatcagccattgactgagcttttggcacaccatgaagagcaattatgtagctttctcattactcttattatccatctgtaaagaaccctgtcaataaactacattccctgctggttggggtctttttttaacctaacctcctgaactttactctactctgcctaaggcaaaaagaggagcagcaaacatcagcacaaatatcaggaggacttataaagtagtgacagaagacagtagaaaagactgaataactagcaagagttcccttcctgtcttcagcactgttacactcttggagctaaacctttcaacaccagatatactgaagatagatttttattatagaggttgcacttgcctttagacagacttcatcatctttagcagtctctctcccaaaattgaaaaagagggaacttttgaaacagtgaaaactgatttattttaccactactttgttttattcatgtcatatatttaggacaattatttctttatggctatcaatagatagtactttagggtatttgatagtgttcatactagcaaaacacatattcatcttgcattatagttgttttttaaaatatcagaaagaattacattttaactttgagtatatgttaatcacattcatgatcattgttaatttatttatctggtatatgtactaagtatttttcagatcaatgaattttaaaaaaacctttatgataattcattgatgatgacttcatcatattaaaattttagttgctgcacattgaattttgattaagatttagttaacccaatctttgggttatttcaagtatcctaagctaggggacttttctttttgtttgtttttaagcttccattacaagtagcgttcacagttggcattgttgacatggaaactagtataaggatgaacacaattaaagaaatcaggggttagagaatgtaaatataattgtaagatcacatatatccatattcccatggaattgtgaaattgctttgaggagtatataaaaataaattcaagcaaaagaaatatattccatgttagctctctagattgacaatttgctgatttctccatcttccaatatggcatgtgattttctgctctgttttgtaatatgtaaagttaatgtgtgatggaaggatacatttctgaagtcacagggcatggaggatggacatatgagaaaccaaaggtagtaatttagtatctcaaatccctgctggtatgcaaagactttagtaacaactcagcccaggttccgactcaataatttccattcccccattgcctaaagcaaattcttctccaccgagtgaaggcacatttagcatccatatagcgttatgttgagaaattcgtaattaaattacattaattctaatgaattcaaaatacagacatttcaaaaagaatttaacgtcctgtttatggtcaaataatattttataatttaaatataataaaactataataataatttaacttgtacattccattgtaatctctgtggagagccttttccccaggcacctccaaataacttatagcaaagattctcaaatttttgtttacatgggaatcatctggaaggcttatcaaaattctgattgctaggcctcactaccaaagtttctggttcagtaggtctggagtgatgccagagaatttgcatttctgacaagctctccagtgatgtctgtgctgctggtccagggaccacacttggagaactagtagcttaaaggaccctccatcctgctaggattggattttccaacaaccagccattttgaaagtttaaaaataagcaaagaatttatataaggatgtaataaggctggagtattgacaggaaatgctttgaatggcatttaatattactctttgtttccagagacttgaaagttcttttcatggataatgctgctctcctcagtcaaaggagttaaccaccagcatctgtgtgactaattgccatctgtgctctctgaaaccttgctaatagaagagcatgtcaaagatctctaatgaattttaaaatgtaccacttgtggatgatttgagattatttttatgtaaaggaaattatagacctcctgtctaatatattaatcaaaaaaattgagtgttatgattgagtgatgcggcattaaagttagattaaaaggtggaaatagtcacataatcaatatagcttgtagcacgtaaaacaaaacaaaacaaacaaaaactggtccgcctgctcagtacccatataatctattaatggtagttgggaaaggactaggcagtctgaatattttgatttataaatcaaataaattgtgatttataaatcaaaaaaatcacaacttctgacactgactttctgtgatgatcacttaacttctcaaacactattttctcattgccacaataagggtcaattatgttgacctgccccataagtaagtgagaacagtaataattggaaggagctctttttagaacttgatttttaaaaataatagtcaaaataccttttagtatatctttgttcaatttggttaaccaatttacctgagagatcatgctatgaaactggagtacaataaagagcaatgaaaaaaagagctgtgttttacatatagaaatgtagaaatagtgatatttgtaaggcttttgctattacagggaggatgtaaattagattgttactctaaacacagacttaaattttgccttcacatatattaacattcgtttgattttccagtgactattctaagtattttagagtggtaatcactgacttcatccaaaccttcatcatcttttcctttatttatttcttcattgagtaaatactagtatctggtgttcttttaaactgtgccttgacccaatattttaatcaaagttgtatgtctcagatacaagatatccaaatatgaaattagaagaaataaagacaaatgaaaagctatctgacgtctataggctacctgatttaaaaataaatgaaaaataataaaaataaatatgtagttcttgtggttcttcaaattgttgatttgctttgcaattaaatgaaatatatatgtattagtctattcttacattgctgtaaagaaataactgagactgagtaattcataaagaaaagaggtttaattaactcatggttctgcagactgcacaagaagcatggtggctcctgcttctggggaagcctcaggaagtttccaagcatggcagaaggcaaagggggtgcaggtatcttacacggtgggagcaggaacaaggagagagtggggaggtgccacacacttttaaacaactggatctcacaagaactcatcactatcactagaacagcaccgaaaggatagtgctaagccagagaaatctacacccatggtccaatcacctcccactaggccccaccttcagcactggggattacagttcaacataagatttgggtggagacaaatatacaaactatatcaacatctttattatatatttatcatatgtccagatgtcattcattcatccatgcacgaatgcatgcaatatgcattcatataaactcattttcgtgtaatcatatgcctacagctatagagcacaccattactacaaaataaattgaagaccaagatttcttacagaaataaagccaaattcttttctcacaaaattttcgataaagttggttaaatactatcagtatcaggctaaattagcaacttgaatgggggtacatcagaagccttaatcaggccaagtatcttgtggtcaaatagaatattagtattatggaacttcaactttaggaaattatagtatgacctttctttagtgtttaacttattctttaaaggctacatgatgaataacatatattgccaattatctatcaaagtcctttaagattatattacatttttgaataaaatctattttgataaattagagaatgatgccttagggcttatttatattataccataaaatttttagaaaccatcattctcggcaaactatcgcaaggacaaaaaaccaaacacggcatgttctcactcataggtgggaattgaacaatgagaacacttggacacaggatggggaacatcacacaccggggcctgttgtgggctgggggaagaggggagggatagcattaggggatatacctaatgtaaatgacgagttaatgggtgcagcgcaccatcacggcacatgtatacatatgtaacaaacctgcacgttgtgcacatgtaccctagaatttaaagtataataaaaaaattttaaatttctcagaaatattccaaatacttattgagcatgtactttgaacagacattgttctaggcactggtaatgcataaatgagtgtagtaagcaatgggcctgaaagtgttcaggcagcatcaatctttgttagaagtgtacgaaatttatttaaacatgcacttaccttttggactaatcaaaggtatcagaacattttctgagcagttttagactctgaggaaaaaatacaaatatttcagagctgtagataatgatatcttatattctgaaacctcagataaaccattaggttacaatgcttcttgaatatacttaccaattaaattttttgaggcaaaaaataacaaaagtgttcttccctgttattaaaactgaacaggtctgtgtcttctaaagatgctatggctttaatcattttaactctgaagataatgtttgctttcccactaaatcaagcctacataatggagcacagaattctgccctgatgaaatgggcctccattgagtaaagtgcttctgttgtttatattcatcttttataatagatttggactactgttttgtcttagaagcttatatgcagggctatccctagacagcattttgaagtgggcaaaatttatgttgattgtgctgtttctagaaccagtcagttatttgctaaaatggctgctaactcgtaatttgtcatttactcagaggtcctaaagcaaactgaaatttaaaaattatcactcaaaaatcacaaaactagtacaaaaaatttaagcaattaaatttcaagaattatttttataagtttgtagcaattatagtacggaagtaattaaagcttaaaactgtactaagctttttgggacaacttgcattatagaaatatagacaatatttagatacaccatttaaatatacatttatgcttttcagtgaataacatataatatgtattacatattaaaagttatatgtcatatatacatttgtataaatatattttatgtttattttagtataatgatatgtacatatttcataaacataaatatactacatatatatgcaattccctgaaattcttccatttgccaagacacctattagaaaaatgagagaaaaaagaagatatgttattgagtgaatctgaagttctactaaaaactttaaagctacttttcatatataaacataccttataaagaaaaatacatttcataatacaaatccaaatttgagagaaagagatcaggtatgaagaaaaaaatagacttttcccaaaaaaactattttactttatagttttatgttctttggaacaatttggttcactttatattatgttataactaacttaatgccatgtattcatggagtggagtgacccaaagctagaattttatttttaattggtcacaatctcatgctgtcaaacatagactctcctttaaaagaaaatgttgaagtcataagtaactatgttgttgaataagcttctaaaatttttgtagttaaaagtatgaattaacatattggactatctgtctattgaataaacacaggatgtcatttatttcttcttgcaacaatttgctgtttaaatgttatgacctgccttaatggaataaaatttcatatattatcactttacattatttcatattctctagcttatctcaaaagtcttattaacattctttatgaccacacttaaataagtaacccatatacaattctatatgaatcataatatacatataaattgcaatctatattttttcctttaccatatctctgaatcagtattggttattctctgcctttttcccccttctttgctatactgtttgagtcagtattggtaattctctgccttttccccccttctttgctatactattctagagagaatattggccatttatctccaaaggacaagtaacagtgtgatgtattggtaagataaaagactagatgacaaaggaaatggtatctcatccatgctgtgttactgtttaacagtgtcatcttggagaaaatcactttatcacttagggtccaagtttcctcacttaaacaaacatacaaacaaaaacgatccagttcacaaagatggactctaaagcccctttctatatcttactgcccatgaatcactttgaaatagtggtttctaatctttatgttgacttcctatcaaaacagtttattgagaagtcttgcaatatccattgattccaatcacatctcatctgtccatgtctgtttactattttgcatctcatgactttataaaaattaaatgtctacaagtgatagtgaaagcaaaaaggttaatagcaaaggttaatagtgctcacgtgttccactagaattaaacaatgattcccatgcctcctccccacaaaagagattttttttccaaaatcagtcacttttaaattaacttaatacaaaacaaatctctaaactgcataaagaacattaactgaagaaaaatatttgaagccattattgaaatattttatattcaaggaaatcacaatttaagaattaatcctgactgactattgtcaatctggtttctaactgactgttaaacttttaaattgacaaattgactgacttaaatctctagactaactctccatccttgatctcagtaaatatatgaactgcagtactgttagccagaaagaatacattgtcctctgacaaagtaggtttgaattgagttcaaggcccctttgctgtgttcaacatacacaccatgacctgctctgcttcatattttgctctcttccttaacctcaacaattctctgccattgatgggataagtatttcatgctgttagtttgaacatagtactgagaaatccaaccaggtcacttctgaaatagcctgcaagcccctagtagcctagcaacttctgttacaccaaagccagactctgtcaaaaaaatctttttaataattggaaaaaagtatagcagggagctagtattaaaaggtaatgtttcctaacatttttttgcatttgtcaagtggaaagataggaatgtatagaaccaagggaagataggaatgtttaagtgacttctaaaatattattatgaaaaagaaactattatcattttttgtttccctgagtctaggaaaagaaaaaattttaagcctcaaaatttggaattaatgtttttgatcataagactttcccagtaagaataattataattatgaagtattttgtatcaattagatattttctaagcatgttgcaggcatggattcttttaatactccaaacaacccaataaggtaaatatgtatatgagccctttatttttaaaaatgaagatactgatgcttagaggttatgtgacttgccctaaaaactttagttttgtagtagaaaccagtagggacaaaacattcactttatagcatgctttatgataagtatctttgggattaattttctggaaaaggttgtggagtctcattagctacttgagacaaaggattatagatacagatatttataaaaattacacacatatacacatatgtacattattttcatctgtgagaacaattacatatgtgagccttctgtgggaagagaacataatcaataagtatgggacaacttggagttacattcacttacttagtactagaaagcatttttctcctcaagcagaaattaagctaataataaaatatatgagatataagaatactgttacaatttgaatatatataacttggaaaagaaaaccagaaatgtatgttttaaaaaaaccaggctgggcacggtgtttcacgcctgtaatcccagcactttgggaggctgaggtgggcggatcacgaggtcaggagatcaagaccatcctggctaacacggtgaaaccgcgtctctactaaaaataccaaaaaattagccgggcgtggtggcaggcgcctgtagtcccagctactcgggaggctgaggcaggagaatggcgtgaaccagggaggcggagcttgcagtgagctgagatcatgccactgcactccagcctgggcaacagagcgaaactcgtctcaaaaaaaaaaaaaagaaaaaaaaatagtaaataaattgaccaaaaattaagggcatttgggggtggtaagactctagccaactttttatttctttatatatttttcattttacttaatgagtaacattactactaatatgatgaaacatatttaaggatgaaagtagaaatgtaattatatagtgttatagtcagagtcttagagttataaatacattatctgtaattatttctagaaagtatgagcatccattagagaaattattaggcagtaatatgcacatttgttagattttggatcttagaaaatcagtgacttcaaaaggcaacttacctcattatcaaaatattagatattggaattagatatcaaatgaataatataaaaatattagaaacttctttatattgacccgaattttatttcaatagcatagtttatacttataaaaatagaaaaaaatcctggacttaaaaaagcaatccaaggtgatgtttggcttatttactagtgcttttgaagtgctactttattcataggataataagaacttatttgaagcttacagagatcaacagaatatgagttattttctcaaagaactctcagactaggtaaagacattagacatatacctaaataaaagtcagaagaaatcattgattcattcttcaagcagtactgaatatctagtagatgacaggaatacactagcgaataagacacagtctctgccctcgataacctcacagtccaggagaggaactaaataacaaatgtagtgattacaagaacaatgtatatatggattcattcttttatcactagtctatttcaagacttttattaagcaccttctgtgtttctaccaaaaaatgggaggagaccgtgaggggtacagaagcatgtcaaaattaagtccttatggaaaggaatatactatccagaatgaagtaggcaactgtttgtatttgaaactattggagagaaaattcaaagcaatttttctagattctaagtggtaaggaaggaaacaaattagcaacctgattaatccaggaacacattcatggatgaagtgttcttgagcattcattttaacaaataaattttaatttaccatattctattctatttcaaagtgatcagtattgcttcaaatgctaatttaaaacattaactttaataatgtagtagtagaatcatctcaattttcctaacctattttcaacaaactgaaaaaaagcaggaagtaataagctaattaaaaataaattgattgtgcttcattccttgatttttgcttccaaaaggtttgccagtggcataacagaacatgactttaaaaaataattaacttaaatactggtcttttttcttaattatatatatcttcatactaacatgagaggaactgtacaaaacaagacagcatgatggaattgaaggaagcctggagcacagatggcatgctgggcttttcctgcctgtgccttgaactggctttgtgatcttgcaaagactactaagctctttttgactcacttagattcaaaatgggaagatttgaaggaaattgtagaaataaattattgtagaaataattttaccctgcaagtctgaaatacttcatttttataaggtaaatccttgacacctaaaaatttgcatgaaaaggacatggtaggtagatggataggtagacaggtaggtacgtgaacacacctatgtgcaagtacactcaacctccagttctgaaagcccttgaaggacctagagggccaaaaacgaagtagatggggacatacttcctgtccacagagcatctaaagcaaacaatcacataatattcaattcaatgaatgatgataaatcttgaatataaaagagaaatagtttcaaatgtcttacaaattgataaatcagtatcagcactgagatatttaaagccattttctttatatctttgctagatttatcatctaaaatattggatactcaatttttaagttgataaacttaaaaatggctttttctatgatgtgagtgatatgcctcatgctgtgtttaaaaaaataataaatccccgctcagacactcatgcaaacatacacaaattgtttcatttacatacttataattatggaagttatctttgaactattgttcaaaaaaacaaaacaaaatattggggaaagggaaacggaattcactgaagttaagagcattcagaaatttgttggtagttttctgatttctccaaaatgatactgtaaagccagagcttgaccacataacaaaagttagcaatcaaaatgttgtaccccttgggattttgataaagagaaagaattatttaattcttaatgcagatttgaattatttaataaagtatttaatagatatgtaatggaataaaatagtattaatagatatgtaatacatatggcaatatctatgcatcttcatatcattaattctgcacagttctgaatgggaaagagtccctagcaatgaattatatggatacaaaagaatggtttggaattatgtggctacaaaaagaatggtttagaaaaataaaatctagaatctaggatgaaaattaaagggatcttatagctctgtaatttttacttgtcagcagtagattctccccctagcttcctgattagaggatgtactttcttggtacctttgttttttgtgcttagggaaaaaatatctatttgggaaatatgaatgtacacactttaatatatggcttattcctttgtatctataaaaatgaggttgttcctacttgccagtagggtctggacagtatactatttctcatgcatagaagaggtggccaactgctgctataccagacatatcttcctaccaagtgcctttgtatccagtgacctctgtgatctgggccactgaacagaggtacggtccacccaggctggccccaactttagccttactggcaacaaatcaatcagttggaactctgaggtactagaacttatagtccctagagctatttactaaataagatcttaatgtgacaaattgttggatttgttttgaataacattagatgtaccaggggaagggtttggacctttcactggctgtgaaacatagggaaggaaatatgccacctacacagggagccaactgagaaacaccaaatgttttatagccatggtttcctaataaaacccctaatttgcaggaagtcatggtgacctttctcgaattgcatgtatttgttgctaatggtctccaacaccatttaaagtggccctataaaaagtataaatctaaaaatctcaccaaaatttggtctttgtttctttaaattcaatagaactcaaagtaaaagagttcataaaaaaattggccccactttcaggtaggaagtgcaccatgtaccatttaaaataaataaaaatcagacaatttctatagaagaaacctgcatttcttgtcagaatatttgtagctatagttagctgtctccaaggtaaataagctaatttttgtagagttgatgtttttctcttttttatagctgtacatggaattattacaaattaagtagggtgagaggaatgctatatctttgctagaaataaattttctaaattcagcaacatttacttatacaaataaaaatggcacaataaggaacaatgaggactgtaaaaaatagtcaataatgcttcttctgatgcaacaaatggaaaatattccatttactctcttaatgattaaatcgtggtataagtgaaatgtagggaaaacagctgggtaaaatgccacacatgttgtttacacattacctgtcaagaaagtaaaaattatctaattgaactaagttctccatacactggtaatctcattccccaccatcaggttgaatggcatttacttcctctttgtgaactcatttggcaatttcaattcagggtctatcttcatctcactttccttcattaatatctaccagctattttccatctttttttcatattccattatttctgcttgagaaactgaaagtaatatttgcaactctgatatttttcaacaactgaaaaatagttgcttcttcttcaaaaataacagcaatttattctatatttatgaacacattttgaaaaatgtttatatttgaagatagactgtatcagtttattttcatactgctataaagaatttcccaagactgggtaatttatagaggaaagaggtttaattgactcaccattccacatggctagggaggcctcaggaaacctacattcatagtcaaaggtgaaggagaagcaaggaacttcttcacatggcagcaggagagagaagagtgagcgagcagaggggaactgctatttaaccatcagctcttatgagacttattcactgtcatgagaatagcatggggagtatcgcccccatgatttaatcacctcccaccaggtctctcccttaacctggggataataattcaaaataagatttgggtggggacacagagcctaaccatatcacagactatattataacagaattgaatttggaaatgtctttcaaaggtattttaataataatatagataatgaaaatgctcaacatatttattgaatactatgtgctgaggactgccaaatattttatatgtatttatctaattccccaacttccctataatataaagacagactatcatttacctaattgtacaaatgagtaaactgaggctaagagtggttaagtaactttcctagggtcaccctactaataaatgagagagccagtatgtaaatttagaaagtcaaatcctagaatcactactctataaaattattgtaattaaaactatcatattgatgccacatatcagttttcatttaagttatgttaagctaaccagataattacagcaatataaggaattctagatatatgacaaacaagtttatagaatttaaattcacacaacagaaaaaacaggagaaaacattagtatgtacgagtgaagactgaacatctttaaggtctataagtttaaaaaagccttcatataagaactcttaaaaaatagaagattatatacagcagtgttaataagcataattcactactcagttagcatggcttaagctttttattacataattaacatgatcattatgtaagagttaaataatacaaaagatttagataaaaaattttttctctcaatcctacaactcatctcctcagaggaaaccactttaaactgtttcttttttttatcattccagatatatcttggtaaatgtaaatgtatgtttattagaaacacgaatggcataaaactataaatattaatgtacagtttatttttaacttaacaatgctccttgaaggtctttccatatcatcccatagagattgatctcattctttttaatagttacataatattctatttcatttatgaaaaatcagtattctagattgtttctagttttttgctgtttctaaaactgctccaataaacatctttgtgtactctccacctaattggttgggtttaattattttgtagatattgcccttcaaaaagattgtatccctatgctcccaccaataatgcagaagtgcatttttcttcatataaataccaatgctggacaatagaagacagtttagtctttgctaagctaacaggtgggaaaactcatgacattaatttgcacatatttaaatatgaatagaattagttataataaagtcaagtatttatgagtagagttttttcatatttgaataggtcatttgtgttttttgttcatttttattgagttttcatctcattgatttgttatttgttttcataggaatttaactactttctgtcacatatgttgtgaatatttttcccagttcttgatttttttatgttttggagtgtgtgtgtatgtgtgtgtgtgtggtcttgtaaataaattttaaattcctatcttgccagaattatcaatcttggcttttatgcctcctaaatttgatgtcacccttggaagttctttttctattctgtgattacaaaaatacacatatttttatctactgttctcaatgtttcatgcttttacatttacttattttatccacctgaaatttatttagatgtagggactgaagaaggaacccattttttcccaaaggttctgcagttgttccagtgccatttattaaataacccatcttgtccctaccaatataaaatgtcacctttccctataataaattcctaaggggagttggacttatttctggattttctgtttagttatattgaactaatctctttcaaagttctccttgtgtcaatattttactcaatgaggtttccttatcttggattgcaaatgtatcctcattacaaccatgagttagaaagaaatcctaagaggtcaatgagccacactgtcttcgcagtaactatacacaaagaagttcagtgtgttttgtttcatttttaatatttcagatccttaaagttttgatagcttcaatgtaagaataattaaagttttgtttttctttccatatagttcacctgcttttagttttcagttatgtagtcttcaagaattctaaaaagatagcagtgctgttatggattgtaatgctatatttaaaaggccttttgcaaaaataggtgtagtaataatttatggaaacaaatcattgctagtttataatatatgattatttaatctttccaactatgtgaaggagcattattaatatcatctaaatttatagggtggaaatctgtctcagaaatgcaagtcattagcacaaggccatatggctagcaaattaaaaaaaaaaagttctcaaatcaaattctgacttttttatagtaatccaaagatctttccactacttcagatttcctgaggttagaagcattttcaaagtttgggctttgtgcctggcagcgtgataggtattgggtagtaaaaacagcttctgttctcactccctatattaagatggaattacttgtgaagtccataatagactaggcagaagcaaaactgaaattagtggttaatcatgacatcctccttgcaaccttatgatacctgcttctcaatgcccagctattttatactctcatggttctaatgtgcaaaaagccaccgcaatgtcctacccaagtctgtcttcctttgacttactcttatgctctaaaccataatttcttttcactttcaacactgtaaatttacagctctggatcttaaagtggtctcctaaaaacccgcgataatttaagagtgatgcaacatcatcctctattctgggcaatttatattttatggaggacttctgacactccattattactttttaattattaaagaaatatatcgatcattttggaaaataagtagcattatgggaaaacttcatccatcatcttataattagagataatctttatgtatatacatgtaacatacttctcgactctctttgacatgcatgtatttattctttgaagggttctactggccataatggttcctagcctcctttttgtttgttttttattctaggatatgtttacaacatcttttcatgatactgaattctcttccaacataattttaaattgctgcatagcaatccacaatgtgtccatctccctgttatgaggcattaggctgttttcattgtttggtgtaatgatactagccccaagtttagtatccttgcagcaaaatctttatccacatatgattattttcttagaataaattctgagcagtgaattcatgaatcaatgtgacacaaaattttgaagctttatgaaataacttagcctaaagaaacaacttcctagtggggcattctggttcatccaactgtgccatctacttatgccaaatatttgcttataagtttgggattatcatggttttgaagagccaaattaaaaccattttatatgtgtaacagagggtgatgttaaaccttttacattttttgaggaaaattttaaggtgagacttgtaaaagaaaggataggactctgggatatggtcacactggttttgaataataatggaaattatgcacacacattattttatgtatcattaaaaagaaaataaaattcagttatactatgactcaatgctttcttatatttttaatttttattttatgattattgaaagaattcttttagatccatttatacatagatttttacaatatatcagtcttgtggaaacattgaaataaaaataaagagataaaagcaaaattgtttaatgcaacctaaaacttcgcattcagattttgacatttcttaaatcactttttgactcaaaattgtccatgtccatatttttctactcagctttgaattctgatgctcattctacaaatttgaatgtgcattggtaagaaaggacagacagctttgtcatgactgccttctggctgacagcaattataagatccaaatctacttttgagacactcaattgtgaaaaatgtgcaaatggtgtatgtgtatatatttcattagaatcaatcaaacaagagtgtagcaaaggaaacatatataagcagatatatttgagaaacataaacaccaaaataataacagctaaagtaatactattcataataatcacaatgatgtattttatttgtgccattaatcattactaatcagatgagattttccaagcatgcatgcatttttggtcaaaacgaaaggtccctgctttgacatacctcctttgccacaaacacaatgagtcagggagatagcaactgatgatgcctaggagttcatctcccatagggtaataacttcatgctgtttgcttcagaactcctacaaattttctgcttataagtattttttccacacacaggaattcatatgttagaaatttttttagtcttccttatagccatccttgagaaaggcttgaaagaaagtgatttacaattataagaatcatgaattgatcagtgtaattcagtttgccaaaatgtatttatctagagggtatgaagaaaaaggattttctactgatgtcagaaacacttgaagatgtatttgaatccagaagaaaaacatgttaatgtttttagaaaggacatagatgttgctggaatccacatggccttggaatataattaagtgagtggtacagtttcaagttgcatgagcatgagattaccttttccattagatttctaaaagtatttctaaataacctagattcactacttatttgctctgctaccatttttaatggctcataatgcagaggcaagctattgtctctgaataatagaacttttaggattatctattcgtcaattggttgcattcttacagaaagagaaaataaagtttattgggaaaatgacatagtacctttaaaatatgcacatgaaatcaagtaccccaaatcagaacattcttaatgttttcaataagtaattactctgagtgcattagtacattcacagacatgatgctgaatataagaaattaaattaccttagatttggtagcagcaatagagtggcgtcaactttagtgagctgccataattcagattagattattatattaataatagtgtattattatattgaaagttttacttgtttctgataactttatatgatattcataacaacaaattatttatcttgaagtattgaagcactgactggaattcctcagcatggatccttcatatttagagctcaatattagccctttacaaaagtattatagctacaaatgtttaaagactctaaatatgtggccaacctcagatttaaaggtgtcaattattacatattatatagtattttttcttttctgctatagcatatagtaaccaaaattgaacatgcaaaaacaaattattttctatttctttgatggtaagcttatctcttaattgagcataggagttatattcagcctatgtcagcacatagactacaaattcaccatcttagttcaccttgtcctttgctttgtgcagagtatagtaagtgctctgagggtgaatgaaattgtgataagcaaagttctaatgattgattcaaatatagaatgtcaccctctatgtttccttatcttggattgcaaatgtatcctcattacagtttatgctgaatgagaaaaggagcaggcccaggtcacaataaaataaatatctatataatttttggaaatgaaataaatatttatttgtaaatgaagtatctatatatttatttgtaaatgaaataaatatctatatgtttatttgtaaataaaaacaaatatctacatatttatttataaataaaaacaaatatctacatattaaatacttattgcatgtcaactgttttatggacctaacctccttgaagatcacattaacactgcaagaggggcattacaatctccatttttcaaatgagaaaatggagtctcaaaaaattaacttttcgaaggtcacacaacttgtaaagaccataacagaaattcagctgtagcttactcattccacgatgccatctgcaaagtttaagtggttttactgaaaaaccaaccttctagaaaacattacattttataatacatacagaaacattaagaatgaaacatgggactactactcaaataaatggaaatataaaaagagaagaaatagcttttaaaaccagctattctgtgagaaagcagtggttctatactgtagtatttaataaggtgtcctagtaaataaacctatatatacttcttttaatagcagatgttattagaaagatctttttcatttaaaacatttattacataaatgatcttcctttaaaataattttttttcacattctatgtggaacatttggggctgccaattgtcatttgagattactcataggtaccctaatttcctctgaaaccagtattacgtgaagaccatgctagaagtgcactgagaggcaaagcagtcaggaatcacaaggaagtggcatttccaggctttctagagtgcagaacagtgtggaggcagcagctctgtgtctgtatttgggcaatggacatgtattaggagaatgttaatagttattcctagccttcatggggtctctggtttaccttaaatattctgttttgttatataacaggctttactgtcttactaattagatgccagctcctaaatacgataatcatttgttggaaggaaaacagttatcataagttatgtcagtattgctgtttatcaaggaaaaaattttatgagtgctatttagaaccataaaattttccttggaattatatgatacccctgcatgtgtaaggggcagatgcagccaaattgcatggagctcttcaagaactgctgaagagcatcttccactctgcaagagaaattgccttttaattagccttttcagattgtctgtacaagccataataattcatttcatcaaaaaatataattatcttaaatattttaggatactcagctggatttaattaagcaagaggatgtccctgtgtttatactcttcaaatggttgctgattccacgtaaactttacaaaagtctcatcttatgtgcattttttaaggccctgaattacttttattacaaattgtgatgccataaaagaaaagaaagccttttctaaaagaagaaaccatcctgaggcactgggagaattaaaacatgagctgtacagtcagaaagacttgggtttgaatttagattccacctcttgctaaatgaaagaccatgggcacgtctcttaatctctctgggatgcacttgagttgcttcctcaataaaatgaagatgcaacgtaccatacagggctgttatgtaaagtaaatagcatgatgcctggtcactccaggtctccaaacgttttgaggtcctttctgtaatagtcacaaagcatactctgccagacatgtagtaagaagattgaacattattctgcctacctttcagccaccactgaagaggccatagttaatgggaactgcattttagctggggcttagttggctcctaaggaagagcattaagcttggctggttctaagtgaaagaagtattccaactggaggaaattaaaactgggcggatttatggctggatttaactgtgggatctgatagtaacctccacctctctacatttacttatatgcccatagcgcatgcctacccagaaaagaagattctttcttttgctgggtccaatctgctgttaagcccatccattgcatttttatttcagatataggactttttcattgctagaatttccatttgcttctttattaaatttttaaaatgtctcctgaaattccctactacttcatccacaatggccatcattcactacagtttcatccactgtattcatcattttctgtaactatttagtgtatgtttaataactgttttaaagtattggtctgaaaagtccaacaactgtattgtctaagaatctgctatttttttttcctattcaaaaggaattacattttcccacttttcacatatctagtaatttttttattgtggatgattctcacctctgagcatataccaatatgttcaataaattgttgataaagtgattattataaaactccatgtaagcctagagttagatctctcaacaatgggagatttgtcttaaactctcccacataacaatttactaaattatatattacttaattgtttttaaggaatttagattaaattttgagtagttatgaaatatatatgacataaaaataattgtataatgaacacccatgtactagtttaagaagatctttagcagtacgtgtgaattcttctccaatctcatccaccattcattccttcccactcagaagtaacttaacttgttattctagaatttttagttactgtgtttatgatttattttcttaattacatatgtttgtgtccctatgctatatagatatagcatatatatgcatatgtgaatatatatatatatgtagagagagagagagaaagagagagagggtttttttcactaagcatgtttttacacttctctttccgctctcaattcaaggtcaccttctctatgaagtcttccctaatcccccaggctccattccctaactagctttgcttccgggcttgctcttccccaacccattcttcaaatagaatctcatcactctcctaaataatttttgtttgtttgtttgtttgtttttgtagagacaaggtctcgctctgttgtccaggctggatggagtggcagtggtgctatcatagctcactgcagctttgaactcctgggctcaagcaatcctcccgccttggcctctccggtagctgggactacagacacctagctaattttaaaaaacttttattttgtagagacagtgtctctttatgttgcccaggctggtctccaactcctggcctcaagcaattctcccaatttagcctcccaaagcactgggattataagcttgagccaccttaccccaccctctttagagacccctcatctctttttgtccaggcttagaagtgaagaatttgggggatatacatgtgcaagctgcagtgctatctgtagttttaaatgttctcaagtaactaaaactcctttgggaaagctttgctactcactgatgggtgcctccctggcccaccagatcagataggagtcttcaccgggctgtcacagagagacttggagactttctgacctccagtgaaagatgaactcagtgtcaatattaacgagggacctttcccccatggacattggacgggtcctaagtagctcctgatgttactctgaaccagaaaaacatggagtagagaaatgccagcctactctgatttttaataacagcttttaggagaagtcatagttttatataatcaatgtaagtaattatcgagtacccaccactctactctgagttctgctttgatcaatgaaataataaataaatgtccttgctcgtgacaaagaaatttatacaaagaaaaaaaatctcaaaagtctttagtaagagtcgtttattctttataaagatatagatcactccaaatcatctctgaaaaatcccttctaaattaaaaaagtattaaaagcattataaatgtaatgcccaactaacttgattggagggaagttttaggtcaaacaaatgatgtaagtaaatggtattttatgtattaatcctataaatatccactttttgggattcaacttaaatatttctaattttaccataggaatttttcagttctgtatttagattttcataatatagtattgtcatatatataatttatttgattctaagttcgtagtcagggacacagtctacaaatgcaacattattcctggaataaataagattagcttgagtgaatgattttattgagtaaataaactggaaacttctatatttggcagaaagttaatctccacatatgtgagtattccaaatgttgctttggccaaagatttagaaagaaaataaagctagtttctgctttgaaagcagttggcctctagagtattagattgattctaaattggaatttttttgctgagttgtcttaaaagttttttcattttgttttgtttttgtcctgtgtactaaagggtccaaaaagattttgggcttctgtttatttatatcatttgacttaggaagcctattagttcttaaatgttctattaggatcttttcctttccagataatatatttctaatacttgaattttagagttgcctgcaactctaggagaaggtggctccttaaattagattttcagtagtgaaaggtaagacttcatcatttcctgtctaaaaccccatattgttgggggcaatttaaagaaatctggagaaaagaattttgcctgggaatcttacgggatccctctctgccatgcttttacctgagccaatatggcaccatgcttttacctgagccaatatggcaccacagtgtcttgctctactcaaattcaagaggagggaacatacaccccacctctcaatggaagagtgtccatttcatcttataagcaaatcacatgagatgagatagaatagctaggtaatttagttttagtgcgggagtaggaaagagaaacccaaattctcaatttttaacacagcgctacagaaattactcctctgaaatcagggagacattactacaggggtacattactgcatgtacctagggtcactaggtacatggacagtaagatcttacagaccaagtataaattccaaatctcccacttcctcatctgcaaacctgcttttctctgagggttgttgagagttaatttaggtaatgaaagtaaaagccttacaccactcttggcacacagctattataacaaacagatcactacatggatttaccagaaatcttagccttctacctgtaatgccttgactaatcttcatttcaaacagggttagttaaaaataattcaatgaccctacccattagaccattcaactactttttaaatttgtaactctgcttatttgctacatgagagttcaacaatggtaatagaagcagaattagaattaaggcttaaattcaataaaactgcatataaaagtaattctatttaatagtttgattgaattttgttaaaaattaaaaaaaaatttgtttaaaaaataatgttccaccctcctttgtgagtccacatgctctctgatagccacagttttgagagtgtagtatgcccaactggaaaatatactactttttgctttgattcattttccccttttcttgactaagcacattgaggggttttcttttgtttttgtttttgtttggtaaaatatccaatatatacattagaattatcttatgtcaacatttgtgaaataagtgcacacttatttttctttggctatatgccaaatggcaatatatgtgtgaattgtatttactgcatatgttttcataagattctctgagcatatcttatgaaatatcataaattggttctagaacttgtaagttaatcagaaattctaggtttcaggaaattgggtatattaattcttaaaatgaaaatcaaagaaaggttgtcttcttcatcttggtccatgtgaacaattttttcttttttttttctttttttttgagatggagtctcactctgtcacccaggatggagtgtagtggtgccatctcggctcaccgcaagctctgcctcccgggttcatgccattctcctgcctcagcctcccaagtagctgggactacaggcgcccgccaccatgcctggctaaatttttttttttttttttttttgtatttttagtagagacggggtttcaccatgttagccaggatggtctcgatctcctgacctcgtgatccacccacctcggcctcccaaagtgttgggattacaggcatgagacactgcgcctgaccttcttttttttttttaatgcagacttcatcagcttgattttagttctgcacaaacagtggtggagggaaaggtttctttggtttcttctgcttttcttagtcactttctttccttggcacctgattcaacatctaggctgtctcgaaaaccctacactgagggcccctcatggcagcaccactgttcttttctgtcctggctccttaccctcctcactgctcattgccctgacagctggctctccctttgaagtactgctcctaaactaatgttttctccatcaggcattaactgacatataacacttgacttgtccttctgaccccacttctttttaaccactttctcaccttattagtctatttactcgtgtgtgtgtgtgtgtgtgtgtgttcaacaaggaagtattctgggcctccttgttacattaaggctgcttactcttcactgaccctaaattaattctaatttggccctttgggactttattctttcctgatgtattagtccattctcacactgctaatgaagacatacctgagactgggtaatttataaagaaaaagatgcttaatggactcacagttccacatggctggggaggcctcacaatcatggcgaagcaggagcaaaggcatgtcttacatggtggcagacaagagagcatgtgcaaggtaactgccctttataaaacaatcagatctcatgagacttattcattattatgagaatcgaatgagaaaaacccacctgcatgattcagttacctttcactggctccttctcataacaggtgaggattgtgggaactacaattcaagatgagatttgggtggggacacagtcaaaccatatcacctgattattaagccaacttcccagaggaaaagttcatgcataggaaaataagctaggaagctgagaatctgctggtcttatgccccacaaccaccaagtgaagccaaaaaaaaaaaaaaatcacaatcaacaactgtttgaccaaatagttcaacaaacaattgagccatctggatatttggtagcgtgttataacaacagaatcctgtttgttttctacagggccatttagtaatttttgtctgtagtgaggagattgttgctttttgcttgtttgaaggacattaaggaaatacactaagtagcattaaacactacctctgtgactgctcttaaagtcactgtgagttggttatgaggcctcaagtgtgcaaatgagaaacacacctgcaggagaatatggatgagaaaccatgaaattggtctttaataaaaacaatgtcctaaacttatgaacctccccaaaaaacctaatcctccttcagtgtgccctatctcagccaatcccatctgcatcagacatattgtaaaagccagaaacctagtgaagcggcatcattttctggggtaaataactggggttcgtcatctcataccaagaaaactaaggacacagacacacacagagagtgagtttaggagtgatagtttaataggcaaaagaaagagaaagaacagctttctgtcttttgagagagagggacacctgagtgggacatttggcctgaggtggagtacaccggattttagagacaggcttgacaaagcaatatctgatttacatagggcccaaagattggttggaccaggtgtgatgtttacataatatgtggggcccctaatcttattatgcaaatagagtctttgcctagctggcaccttgttacctgctccttcctgtacacggggctggcaaagagaagggaaggtggagccgccattttgaacatacctagtcccaggtggccttttaacctgtataaacttctagctagaatgggatggtaaaaagaagtgtgtgcgcgtgattttgcctcctattttgcacgagagaggttctctttttatcctattttgtagacatggcttatctacttctctcttgatttagtctttctaacgtattatagtggagataagaagtttagcaaagttccctgaaggaaggagacagggttgagaagacgggagagaaagacatagtgaaatgttcacagaagaccatccagcacatacctgattcccattgctgtgtatacactttgtaaatttggacaagttctttctcctccctgagtcttggtttcttcattttcaaatgagggtttcatattagataatctctaatgtctgattgtctgactcaaacatctttcgtgactaaactttcccgaatctatcacactaattcatatccttccacatgtcccaagttaaagtacgactattttcacaaccaatgtatcaaactcttcctgctttgacaaaaacagatgcaactggatgtgccaactgattctttgaaaaggaaactcaaactatttttgggggggagttctgcaaagaacattgtgactactgagtttttataggcttttttttttccacttagctatatctagtcttatctagacatcattgaaaatatgcttgaattgcaggcctttttcaggtgtccttgaaattactcagaaccggtaccttccttgacacttctttttattaaatagtataggtggtgggactatatcaggagtggcagacataaatttccataggggccccactgcttatactataaaagctctttttgcagcagcatcttcagagaggaggtgagataccacatggaaatattgctgcacccctaagattcttgcataagtcttgtatgggtgcgtctgctttagatagaacttggagatgtcagtcacaattattaccaaagtgaatagataacactaactcagtcttcctaagagaataccagaaaagattaactggacagattttacctcattcttatggcatgaagttattttatttcaggaaacttaaattctttgttgtactaaattaaaaaaatacagttttgcttagggggaaaaaggaaagaaaaggaatagtagttgaggatattgaacattttgaacacgtatctctcttaggtcttaaaaacttatggatcatggtagacatatttacgagtactatttgaatgtcttcccaagatggcagatcacaggttttacatacggtgggagaaagaaaaagacgctgttgagcagaggaaataatacatggttaaagaatttctaccagaattcattgagcaccaaatattgctgaagttagatattgaaactatctcatggtctattactttgcagtcaggggtttccttatttccagcacagaaataactatttttagctatacttaaacaatattacaatgatagtgtttttagggaaggcaagtgtgaaatgtaagatagaatttgttggtttttttccctgttggggggaatttccaaagaagccaaacaacaataagaattaaaatgggaaatgtgggcaattttctataatatcactgaatgctttctgtctttgtttgtatattgttggaagctgtacttatgggtctattatgactattcttcattggccactgagattaatttccctttataattttgtaattacatttgtgaaatacattttttaacacctaatcaccaccagggcactcttaagagatgtaattagaaagcccatcagacattctgtcttcagtgtattgctaattgtgttaattcatttttttattgcaacttgctgttgatatcatgaatgaagccttcattatagagaatagttctcagtgaaatggcattcccattggtttaagagccttcttttaatttgagggttgttccttattggggattattgtcaaactttttaaaaagtatatttccagttatctgaagacagattgaaagcagacccaagcaattaaggtgagacaagtataacctccctctttcccctactattttcttattctgtactccaaagctacgccatagggaacattcctatcagcaacagtgcacaaaacacagaacaagaacaagatagttcatttcctcttttcttttttttcaaaagcaattcccgtcaatgttgcaaaaattagaaaatgcaggcaagcaaaatcaaatcaaatcaaatcaatcactttcaatcccaccatctggagaggttctagtaccatctttgtcaccttgctgtggaaacttgtgcaattttcctctgtgtcgtgtgtatgtgtatgtgtgtgtgtgtgtgtgtttctaattatttgctttctccttcctcagaaaaagcatatgtattcatgatatgttaactttggccttaatgttgtaacttgcttagggcaatagggtgtgagtagttatgtcttacctcatctgagcaaagcgttgctcccctggcaggaccatcctctgccctgagaacagcatattccagacgagtgttgctccatcagccctggtcacagagagaaaatcatgtggagcagatccacagagggtgcttcagctgccccatagttatcatgtaacttgaggaaaagaaaaaatcatttcttttaaaaaactgagatttggaggttattttttacatagcaaatctagtattttacctagcctctctaagactcagttttcatggctgcaaaattacagtaacaataatagtatctacatgcgaaggctattgaaaagattgaaggaggtaatacgtgtatactcatagtgcctgctgcataatattatattcttcaagccttttttctattcatatataaatatatatttacaaaaatcagattatgtcatacattcagtttgtagtccctttttaaagtaataacataccaaaaacatctatctagataattaaatattctagtacacaatttttaatggctgcagagtattccattagtatcggtgtgccaaagattaacaaattcattattttggacatttagattactttcaaattccactgcattagacaaagttatgatggacatttctacaatcaaatctggaccaatttataatatcaagaggaaaaaaaccttagtcaaagggtctggatgttttaagaattttggcatatagagttgtttacagcttacaaacttataatgttaatacagcgaaacttagccagaagatagttatataatctttaagagtagatcaattaaagaaaaaaatcattgacaacttattttcaaacttcattattttcaatgggcatgtttatttaaatacgtatgtttctagaatcagtgtgaaatgtggaaagaaaatagattttgaagtaagaactgttttcaagttctgtctctgccgcttattgactgtgagatcttaatcaagttacttaacttctgtgaacctttatttccttatatataaagtggagatttaaatgcctatttgcatttcataggaggataatgtaatgtgtataaattgcctggcttctatatgcattcagttcattttagttccttccttcctttcccactagttatcccctactcccaacccctcttcaaaaaaggagtccattttaatgttttaatttggccaaagactaccatatattcttgtgtatcagccaagaaggataacagacagagtttcttgataaacattaggtttcatctatatctttccaaagaatatcagaattttctctcagaacagagaacaagattggagctctgtttcctgtcacccacctggcagcaattagttcaggacccagaacctgcacattagcaacagcagagaagcagtgaacttaagttagagtcaactgtcagttggagaagctattgttagtcagggttcctttagggaaagcgacagatcctgctcagattaatttagtcaactcagttatttcaattcagctcattgactttgcataaattctggtttagttctgcactgcgtcctttagctcctatcactgaatgcgtactaccaccagagagaggtgaggagatacaagatgtcttagaaatatgagtcagctatcagaaaaatatgaatcggattacctatggatctaagtcatgtagatgcctctagacacgttctgaaataactgaacttctcagttattatcccatctgcatccccaaacaaaatcacattagttcagattaattctttggtattcctgattaagtttgtggggttgattaaaccttgttttatgtgctgtaattttttatatacttaatttttaaaaagtgaactggtgatttcatcattgtacctcataaatttgtaaaattataatgtgccaattttttaaacgtgaactggcaaggtgagtcatatgcaagtttctcacaaggactaagctttcttgaataaagcaggatgttcagttcacctgaaaaattaaattgcacattgaaattgactttaacatcatcattttagataaagttcgtcagatcagaccactgattcttagctcttagggtcatggtgggaaacaactgctatgatgagaactatttgtatatcgttgctgacatttagagatccaacttggttggccatcccttatatcattaatctcacttaattgctctgtttttcaagggaatctagacaattttacagaaccttttgaactacttacacagctgtgaagaaaggataatttccaagggcactcaaatttcaggaaaatggattcttaaataatttaataggaagaaaaggaaagtttgtaatgattaaattacatttgatggtgcttttctttattagtaaatatatgcactgaaattggaaatgcaggagcaagcttgtcttttataaggttctagatgaaatatctttgtatgaaatcccataagtggtgagaggattgggagatatgtgtattaagggggtgcatgaggatagctcccattcaagctttctctgcaatacagaaagactccgtaagagcaagtcctggatttggattcagcaaatatttgatgagtgaatttataaacacagtaagtgctctgcttcattctaccccacctccatttctgcaaacacaaacatacaaagataaatgtatgtacaatgcatagcaaggttatgggggagaggcagaaaattaagaaccccagactggtttattgctagtaagtattggtacaacagggcctgtggctaacagccagcaattgttctaattggtggatgttgcagtggcaacaattgttaaatacgttgaacaacaccccagatgtccttttctttaacctcattttattcaagtctaagcactttctttcttaaaatagtcttgatatcagaaccaaacagaatgttaaaggtgactaagagaaattaaactgggcaagaggatgaactatcattatggttatttctacccctactagaccaagctttagaacttcactgcagaatattgaagccaactaggaaaaatctttaatgcgagacagattagtaatttagaacatgaactaggttggaccctatgatagagttttaattagagctcttccactaactttcaaacagatttaaaataaatcattcagtaggttttcttttgctcatctatagagtgagaagttggggatatttgaattctaagatcccttccagtttcatcatttaataaaatcagttgtactttattctgaaagaggattttttcaacagtgtgtgtataaggacaagatgatattttgtataaggactatatcttaatcccaccattttaaattcctgttggtatttatagaatggtttcgtatgtatgtggcattgacttctacataaatattttaaaacttggcttaaaagagtttataacttatggcaaccaatttagttccactcagtaagtctcctgtgtaagatataatagaagggtaattcatcagtatttgtgaaagtggactttcaaaaccaagcaccaacatatttaaccaatcatatatttaccagagtttcacttagccaaacaagcatggcaaagtcctgcgatttcttttccaatataataaagaaatgctgataagtctgcaacatttaccttttactgcagctttttctgctaatcaagagaccaacatacttttcattcaggaaatcaatggaagtcaattattacccagacgtggccatccattccttgaatgtccattgcacattaatattggtaaatgtaattagaaataacagctaatttaaatgaatttatttagaggatccatggttgcagaattttgcagaacttttaatatcctaacatacttgcgaatttccagagaagagcacgtggtatttcacatttcacaaactttgttccagtggactgctcattcctctttttgttattaatgagtgttcacagagatcatattttgtaaaaccaatgtggaaattctgttttagtttattttctattacaatgtcaagtagagtcaatatttatctatacattgctatcttgtctctgtctgccaaatacatgttttgtattttagtgatggttacattagagcctatcttggagtgaaaaagacctttaattatagtccagttgtctaattttataagtaatgaaatgcatgcccagaaagatatagtaacatgcttaagagtcatgtggtgagtttgaagttcttctgggattacaatatggatctcttattcctttgcccactatttttttttccattatgccatactgacttttcttctaatatttgccatagttgatcttaacagaagttgaaaagttacaaggaaaccttgaacatgctaattacaatcagtttctcaaaatagaagacccttctgataattcaatttccatttgtccaaaactgagtttactttattcttcatatatgacagtctatatgtagtgactaagaacacagattttggaattagcccacttaagatttcttcagtatgcttagtgattccagtatcttcatgcttatcctttagattggattatttttctcccaatctctcacatatcgaagtttggcttcatcttaagaaagcattgaaacacatctaataaatgcatgatgtatttttaattaaattgatttgaatcaaagagtttctatctcccctttatcatggctgctattagagttccgatactataaagtatacgtgaggttacaatgcctttttcttaaattagttttaattagacagatcataaccaattatccattaataatagccatttgggtaatggcacaattctgaactgcacacaattctgaaccatttctctaagatctccttgtttattaatgttgcctgaccaactacttagatgtcatgtatttgttcattcattgaatgcttattgagcacttactatgtgcaaggctttgtgttgagtcttgtatttaaccactctgttacacaggatgtggtactcaaattcgaagtaaagtctacttatttttgaaaacttccaccaaactagtatacatcattgcttcattttaaatcttatatcattgttttgtttcccattagcaagtataattaaagtcacttaaggagatgggtcttagagtagaaattctgacaaaaacgtgaccagtaacttttcaaaatttgtcaagtagtttacaccatttccaccagggttaggctagcatcttggagtagaaatgtgctttctagccatttccccttaatagctggaaatatatatccatgctggattattcctaccagggatttctggactgtgggcttttggtctttgaacttctaggcagtgataataaaaacatcatctatgtgcttaaaaataacagtgaaaataacatatgagttgataaatagatataatagagatcaagagtgatttgagactgggttctccatggaaactgaaaagatatgaaggacatgctgttagatcttctcctcatttataccctcaaaaaacatgtattgaacaagtatgatatggaaagccctgggctggttgtagtcagggatattgttaaaacagaaatagcttggtggttggagagaccactctgagggcacaggactaggagtcagaacacctgatttgtctactcagtaacttcatgactggaaactaacctaatctcctaatctccttgagctttgagttgctcatccacaaactaagtgtaaaaggagaatctacaacattttgtcttaagaactaaatcaaagaacatgtatgtaagaatctagcatggtactgaaaacacagtaactattaaatgctaattagatgatgtatacacataatgcaagctacaaagtggttaagtttataagagctcaatgtgctcaataagagctaagttagcatgatgatgtaaaaatacatagctgagtcaggtcattgacatgagtaaaagaaaaagtcttatttctttcaatatgacatcatttttattctgtttcccctatcatctgaattatttctcttacattgtgtgtcacactgtttacccagaacagctctccaatgcagaaagtgccaagtctttctaccagctagacttggagcctggatttagatagacattttaaactcatgaggagagtaggaagacactctgttttgctttatgtggcaacataaattgggtttatggccatattatgccttggcaagttagttcctgtaataatgaagaatgattttcaaataggttatataatcaaggagttatccaaacagaaggctttgctgtggcagcaaaccagcctatcctgtggtttcacaaaattcatttgcttattcacttagactgcatctacagaaacatattaaatagaattgtctagaaagaaatgccaaattttgtatttgaaaagattctaggccttgggtgacaacagctcatagctgttttgagacaagccatgttccttttcactactctttgcccacattcctcatcctactccttgttctcatagccaagatattggtggaaatgttcttgacaaatactgctccaaaggaataatcgcctgtgagatgcccattatagtactgggctttgtaccaaggctagagaatgttgaaggcctcagaaattccacttttgcttcatgaaggggaaagagagaaaattataagtgaaagggcagctcgttctcatgacttgcaggcttaatcaactagagtggtttagaaattgccattgagctaaaagagcattttccaaaggatattctatagatgggttattaattgttatcacaccataaaagtgtttcatggtcaatcaggtttgggaaatgaagagttaacatcaaaggggtctttttcgtacagaaaaattagagtctttaaagtgctaatgtctatagtaaatctctgagagaataggaaagacttaatttcctaaacttatttggccatggaaactttattttatgctgcttctcatgggattgctgctcttctatggaacatgcttttggaaatgccaatctataagaatagacacattattttcagacattgacattttgtactctttccacaaaaaataaagataatttaactttgcttttcaagtaaatttgttccccttacatgaagacattctgttcaatcattatcattgatttactgattttcattttatttattcacagaataaatgattcccactgatccacccacttttgccaccccaggatgcaattttctggagagaagattagtggcaagtgcccatttgtggatacaaaccagtatagattcggttgtcccaccagtaactgaacctcttacaatggtttcttcatttgcatgttctaaatcagccacaacaatcagatttttaggctccacattgatctggtttagggtctgcattaactctggcaggatggggctcctgtccaggtttttgtttgaaaaaagtggttaaaagatatgccggaaatcattaccctaaggagccagattactgagacaggtcttgtatacttctcctattcaaggagggcatgactaagtagtcattctatagataagaagctggtataaaagtgaaactctttgttcaaaaataataataataaagggagaagcagaaattagaattttttggctccgcgagtagggcactttaagtaaagaacaatgcttcctgtcttactaaattatagattgacattccttaggttctgtccctaaattatcccttaaaccaagcaaccaacatttactagcatttactatgttccaggctcttctctaggttcttgggttgcaagagtgaacaaaatccttgctatggagatggtaattcatcaagaagcaagtaaacaaataatcttatgtcaaagttaaactttgttttgattttctttgagtgatcttcctggtgtaccagatgattttctggttgcaggtcttctgtatcacctatttccattccaatttgaaagttctcaaggtgcatttcatacccactaactgctgtttggtgatactgtcctttctacctgggtccaggtttcactctctaaggctcaggtcagctattctcctgaaggtcactgaactgtgaacataacaatatcacctggaatcttgcctgttttaaatcctacactaatcagtctctagattccacccccaatctatccatcctttcattctgtcttttaatcatctcttcaactaagccattggcctctgtcagttcatcccggattttccacagttatcactctcattaactctctctcccattcttttcttccttctaataatctcaggaaagagaaattatctcattcttcagcctgattctactcttccttgcctgcttttctctctcaaactgataaagcacctcctttcttcacagctctacctgtatatgttttcttctcccctcctcaggagtcaccataaagtgctttttttcaaaaaaccatatatctacaaggcattgtttaacccaggacaactgcagtgcaacttttctgactagaggcaggcttggtgagcagagatctactatagtttgggcatgaggcatttgttatagttccaaataatgcaagtctttaaaattaagacctcttcaattaataggcaaaacaaaagaaattgtttcttatctaaatcccctctgttgtttccgaatattcactctcagatctgttttcatactatttttgttctcataagctgtttgtgatacatgtgtaaatgtcatgtgtggggctttaagaccatgggggtccccttttctacagcaagatgactgtgcagcccattcaccttaaagtgacaagtctcccattgcctccaatgctcctcattttaataacactgttgagtcaaacagcaacttctacattgcaaaatatcaaatacatgacatggatggtaccatgtactatcaattaaccatccaatcaccacatattcaccttcataaagaagctggtttctcgccatattagctctcacatttggaattaatgagtaggatcatgtcaagaggagatttggcttttaattctggcaccacccgtggtgttctacatatccttcttctctcagaggctacattacaggacaatcatgagcagtcacctttaggaatttaatgtatgtctatgacttctttgtactttgtaaaatgcaagatgtagacaaagaaaactgtgtcagtttcacaagtatgtatacatctcaaaaactccaactttcacaccaggaaataagtctcacccaatatatggaggtagtgtattcccaatcaacaagaaagacacaaattaaaagtttgcctcctccacctactttcctctcctgaatttcttatctgtccttttagcaggccacaaatctaagagacaattttaatttttctctatttctcttcatgatcacactggtgcttgttactttaagggggtcctgtattgtattcctagccaatggttctttgcactaacttaaagtttgtagggaattgctaaagaagagtaaaattgcactcattttaatatagaacacgagttggagggattacaacttgaagcagggaaactggtctagaggctgttgccagggaactgaagagaaagatggtgacctgcactaaggtagtagataggatggacatgtttgaaaagatcccaaaatgcagatggaatcagagagtcctagagactagaagcttggaatgaagaagtgagtggaatcaggaatgatcccaactttctcattcatacaattcatggataattgtggaagacactgatatgagaaatgcaagaaaatggacagagaaaataatacatttagatttgccaatgtggtggtgtggtgaggatatagttgtaaaaaatcagaattccatgttttatttaactcattagatcacaagccaccatcattatcaataataataaacatgtgtatgcaattttgtgatgtgccagagatatatagctatctaaaaaataattcttatctgaaagggtttagcattaggtaaggttgttaggatgtgtatttatgtagatgaaaatacatagaaatacacagaatgccaaatgggtagtttgggtaataaatttcctaagcactcaaaacctacttttttagcccaatttccacttcctaagaggctttatgctccagctaaattaaactattatattcattattctttagttatcctaagttttcctagatccatatgttgtttttatgtgctttctctgactaagaggtctttgcttttcaaaaccctattcaggttttcaaattttatctcaaaatataattccctccctgatcccccaatcaaaaattatataaccatcttataaggccctatatttttgcaatttcattgaacctttcacccagtctgcatttgagttattttgccacatcttatatcatttatacccttccccactcagatgctcaacttcttatactggaatcatatctcatccagctcatatttacatttcatgtagtgcctcagcacttaatacgtgtgtgcgtgtatgcttctaatttactgagtggacttgaaataaatcagaaacaatgaataatgatcaacatataaaatgatgtttcaaaattataatttacattctcaatttcctgttatgccaataggtaaactgaatagaatttacccttttggggacaagtataataggcatacattgtagacagtaatttatatctggaaaaatctgtaaatactttttttttactatttttgttttttaaaatatgtgagtgcatattttatttttttctttttaaaaaataaaatttttacacctattgacctgtcctctaagtttcctcccttcgcacccctcccaccaacaggccttggtgtgtgttgttcccctctctgtgttcatgtgttctcattgttcaactcccacttatgagtgagaacatgtggtgtttggttttctgttcctgtgttagtttgctgaggatgatggctttcagcttcatcctttgcagggatatgagctcattcctttttatggctgcatagtattccattgtgtatatgtaccacattttctttatacggtctatctttgatgggcatttgggttggttccatgacattgttattgtaaatagtgctgcaataaacatacgtgtgcatgtgtctttatagttgaatgatttgcattcctttgggtatatacccagtaatgggattgctgggtcaaatggtatttctggttctagatccttgaggaattgccatactatcttctacaatgattgaactaatttacattcccaccaacagtataaaagcattcctcattctccacagcctcaccagcatctatggtttcttgactttttaataatcaccattctgaccggtgtgacatggtatctcactgtggttttgatttacatttctgtaatgatcagtgatgttgagctttatttcatgtttgttggctgtgtaaatgtcttcttttgagaagtgtccatatccttttcccactttttgatggggttgtttgctttcttcttgtaaatttgtttaaattccttgtaaattttggatattagacctttgtcagttaggtagattgcaaaaattttctcccattctgtagattgctggttcactctgatgatagtttcttttgctgcgcagaagttctttagtttagttagatcccatttgtcaattttggcttttgttgcaattgcttttggcatttttgtcatgaagtctttgcccatgtctatgtcctgaatggtattgcctaggttttcttctagagtttttatggtttgtggtttttatttaagaatttaatccatcttgagttaatttttgtataaggtgtaaggaagaggtccatttttagttttctgcatatggctagctagttttcccagcaccatttattgaataggaaatcctttccccattgcttgtatttgtcaggtttgttgaagatcagatagttgtagatgtgtggtgttatttctgaggtctctgttctgttccattggtctatatgtctgttttggtaccagtgccatgctgttttggttactgtagccttatagcatagtttgaagtcaggtagtgtgatgcttccagctttgttctttttgcttaggactgtcttggctatacaggtttttttttgatcccatacgaaatttaaagtagttttttctaattctctgaaaaatgtcaatggtagtttgatgggaatagcactgaatctataaattactttgagcagtatggccattttcacgatatcgattcttcctatccatgaggatggaatgttcttccatttgtttgtgtaatctcttatttccttgagcagtgatttgtagttctccttgaagaggtccttcacatccctctttagctgtattcctgggtattttattctctttgtaatgattgtgaattggagttcatttgtgatttggctctctgcttgtctattgttggattaaaggaatgcttgtaatttttgcacattgattttttatcctgcgactttgctgaagttacttctcagcttaaggagttttggggctgagatgatggggtattctaaatataaaaccatgttgtctgcaaacacagacaatttgacttcctctattcctatttgaatacccttcatttatttctcttgcctgattattgccctggccagaatttccaataccatgttgaataggagcagtgagagagggtattcttgtctatgctggttttcaaagggaatgctttcaattatccccattcaatgtgatattggctgtggatttgtcataaatagctcttattttgagatatgttccatcaatacctagtctattgaagtttttaacataaagggatgttgccttttatcaaaggccttttctgcatctattgagataatcatgtggtttttgtcttgggttctgtttatgtgacggattacatttattgatttgcgtatgttgaaccagacttgcatctcaggatgaagccgacttgatcatggtggatacgttttttgatgtcctcctggattcagtttgccagtattttattgaggatttttgcatcaatgctcatcagggatattggcctgaagtgttcttcttttgttgtgtctctgcccggatttggtatcaggatgatgctggcttcataaaatgaattagggaggagtccctccttttcaattcttcagaatagtttcagaagaaatggtaccagctcctctttgtaactctggtagaattcagctgtggatccatttggtcctggacattttttggttggtaggccattaattactgcctcaatttcagaacttgctattggtctattcagggctccaacttcttcctggtttagtcttgggaggttaccaggaatttatcaatttcttctagattttctagtttatttgtgtagaggtgtttacagtattctctgatggtaatttgtatttctgtggggtcagtgatgataccccctttttcatattttattgtgtctatttggtgcttctctcttttcttctttattagtctagctagtggcttatctattttttaaatttttttaaaaaaccagctcctggttttattgatttttttggagggttttttgggtttctatgtccttcagttctgctctgctcttagttatttcttgttttatgctagcttttggattagtttgcttttgcctctctagctcttttaattgtgatattagggtgtcgatttgagatctttgcagctttgtgatgtgtgcatttagtgctataaatttccctcttaacactgctttaactgtgtcccagagattctggtacattgtctctttgttctcattggtttccaagaacttcttgatttctgcctgaatttcattatttacccaggagtcacttgggagcaggttgttcagtttccatgtaattgtgtggttttgaatgtgttttttagtcctgagttctaatttgattgcattgtggtctgagagactgtttgttatgattttagttcttttgcttttgctgaggaatgttttacttccaattatgtggtcgattttagaataagttccatgtggtactgagaagaatgtatattctgttgatttgggttggagagttctgtagatgtctattaggtccacttgatacagagctgagttcaagccctgaatatccttgctaattttctgtctcattgatctctctaatattggtagtagaatgttaaagtctcccactattattgtgtgggagtctgagtatctttgtaagtctctaagaacttattttatgaatctgggtgctcctgtatagggtgcatatatatttagagtagttagctcttgttgaactgttccctttaccatcatgcaaggccttctttgtctttttttttatcttgttggtttaaagtctgttttgtcagagactaggattgcaacccatgctttttttttttttttttttctttccatttgcttggtaaattttcctccatccctttgttttgaacctatgtgtgtctttgcacatgaaatggatctcctgaatatagcacatcaatgggtcctgactttttattcaatttgccagtctgtgtcttttaattggggcatttagcccatttacatttaaggttagcattcttatgtgtgaatttgatccatcatcatgatgctatctggttattttgcacaacagttgatgcagtttctacatagtgccattggttttatattttggtgtgtttttgcagtggctggtactggtttttcctttccatatttagtgcttctttcaggagctcttgcaaggcagaccaaatggtaacaaaatctctcagcatttgcttgcccagaaatgattttatttcttcttcgcttatgaagcttagtttggctgaatattaaattctgggttgaaaattcttttctttaagaatgttgaatattggcctccaatctcttctagcttgtagagtttctgttgagaggtcttctgttagtctgaagggctttgctttgtaggttactttgcctttctctctggctgcccttaatattttttcattcatttcaaccttggagaatctgatgattatgtgtcttggggttgatcttctcatgaaatatcttagtggtgttctctgtatttcctgaatttgcatgttggccagtcttgctatgttggggaagttctcctggataaaggataggtaaattctatgggtaatacagtagatatagtgcaacaggaacttaccagttaagatacagtcataaccactcacccctagttggaatgtaggtttcacacaactcccactgatgaaaagaaatatatgtatttttcaactgtttaaccctttgttaagttttcttgtgtaaaattatctgcagagccatgaaaaaccatttgatatttgtgactaagcagcctgtttggatgattatgctcttcagtatgaatggtgagctgttaaatgacatgctcaatcattgctatggaagaaatttgttcttactagcaacttgaagcttaaagaaacatttataggaaagaaaattactcaaagctttaaataaggctacttttagagttggccttagactacctagagggcatgatgattaatctttcacaaattacagattttatttgttcatgtccagtgaggtgacttcttggtggacatcttcattgcaattttcagcagctctatcaatgacacatgttaactgaagctgacatgggttgctcttgctctcttggaatgtctttatttctgtcctaatatgcaaaggtagtgccagaatttcttaataggagggcctcaggtataacaatctagttgacaggaaaagcaatggaatcttcactgcatttgcatcacaagcatactgttttttcttacgtgtgttttttagggtgtcttgggatgttgatcctctttaagtcaaatagaaaaaatgaaaatgaaatgccatagccaatattagagatatattaattttagtctttgttgcttttatatttttctaggacaaagagatcttcaaaaatcaaaaatgaggttcacatttacaagccggtgcttggcactgtttcttcttctaaatgtaagtaaaacaaaagtattcaaagccatgctaatgaagaatgctgaataaggaggaatgttttgagtgtgatttaattagtatttgcaagggtaggcattggtctccagaagccgaaactaagttcctcaaagccattattttgcttaatattttgttaacaaatctctcctctgaactgtctctaatttaaattagtgtttgtggtctacccagaaatttgagttttgtttacaaaactttagcattaattttagacatctccatattatcttagaaattaacatagttggtaaataatattctcaggtaaaatgttttcatgttgaaaaaactatgttttgaatcttagttcaaaactatgatttgaatgatagtttctgggctgttcaatccttttaaaacatctgggctgttcaattcttttaaaacttctgaactctttcagtctggctattgatttcaaatgtatttttcatcaaaaactgactatggaaacttcaagtctttatataccactattattctataactctttctccaatcttttaaattatgttcagtttgtgtaattctatattgttttattcctaaaagatttagcaagtaatcggaaatataatcatcttagagaacactatcaatgcctattatttgtgtcaacattgttaacctaccatcacatcagcttcaatagcacatgaatgggagatgagtaaattttacgtaaatcaatgtggagctgccttgcttcttatcctttctgtcacatcagagtaacatttgctaataaattacagttggcagcaaagatcactcaatatctcatactgtcatactgacccacacaaagcttgcatagggttcgaggatttccattgtttattaatgtgtagttataggcctacaagtctgtctaaactgtttgtctttatattattcaagaaaaaaatattgctagtatttcataaatgttaatttaatttgtaaggcaatttttattttgtaatattgtacatcaaataaatggtgtatgggaatgactattaatgcaacccttttgcacctatttggataatgagtataacttattttattgtcagtttgtcttatcattttatcctattaaatatttctttagataaattacagtaaaattttctaaaagtaattagtatttggaaatgcaggtttctttatgtcactggaaatataataaaaacactaaacaagactactatggaaactaatagaatattttaatttgccagtaaatacccaggccttatgtggtcttctatggatgtaaaaatattgctatatattattgatattgttatttttcatagctcaaatgaaatcctaagtgatctggtatatgtgaatatttttgaataatcatgcttcataaataattatattgaacatgacagaagaaaaactaaagaaagaactaaatactaacaaaattgtttgaagattatctaattaccttttttattacaaaggctttagaaaatttgtagtgtgttcaaaaagcaacgtaggtcatttttattcaaaacttttactaatgtagtctataatttaaatattgaaaaaaatgcacatctttgcatgagccatggacagaaacattttatggcttgtgggcacctcttgaccagcaagcataactgaacagggcacttaaatgggtttgtgctctctttgctgtgacagagagaaatttgcttctctctctgtatttggattatatatttgatttggaggatttgtccataaaacaaaatttgatatgtccattttcttcaaaacgtttattatgttcacactgacacttctcagagaaaaaaaattttaatgacaatgttcctaattaatccgaaatattactttttagacaatagattgttgtgaaaatacactaactattgaactctatcaactcgatttccatataggttttaaagtctcagtttgggtcaccaccttcaattagcctctttccattaaccataaccacccactcaattcctctccctcctgcgaggtatacagtaagcttagagcagcagttggtatacagtgagctctcagaaaaagatgccagtgacgatgctgatgacaacaaagccaatggtaataacaaagacataaaaattaggttcttcagagcaggtgttattgttacttgtttacttatctatgttcacctatttgctttgaatttatgatatatgcagtttcgttgtttatataatgtaagtagaattacacagtacaccagccaggcatctgatcttacctgggaaaaccagggctaacactttttacaacagtgtgctttgtcctttcttattttgaaatgtgtaagtaactgaaaagcaaaaataattatatacattatcaacattaaggatggtgacatgaaaatctcattggttgcatcagtttgtggattctaggtttacatactttttagagtagttttgcatgtggcaatcaataggaagctctcccttcagcacagcagcttgggtccatacactcagtgggcttgaactattgcagagatggaccatttctcaaagttccatgaagaacagctctgtaactgtctctgtacttctagaaactgcctgtaacccaaaagcaaactgactgtcctacatcacagtaggaattgcataagcatatgaccatttcaaatcagattcctttcctgtggattccatgattagtgtggataaaatcaagaatgtaagctctatgagagcaggtggcttgccagtttggttcactgctgaatttccagaacctagaagagtgcctgacatgtgagaggcactcaaagtatttgtcgactgaaaaaaactgatgatgtttgggctgctgactactgctgttggtggcagcttggtggtcgtatgtgtgacgccatttacttgaacctttaggagtgacatcacatatacggcagctaaactgctacatgggacaacaattaagagttttgtttgtttgtatgttgaaaaatcatgtaagatggtttatatgcaaagctttgaaaactgatgtttattcagaaaaacaatatgcccttaatatctcatttctatctgaatgtgaacatataatcatatttttatttaaccccctgaaataagtagtatagacataacacttataataagtaggatatttgaagtctcttacattgtttggtagtttcatccaatcactactggttttattttgtctctgtattgctgaaaatgataccaccttcccagcaattcttatttgtataaaattatcagcaaaagtttaacatttaataaactttatgaaatttgtatatgtgttatttttaagcagtagtaattccatgattgtaattatatatatataatatacataaaatcatacatatacatatatttatatgtttatatctatctatctgtctgtctatctatctatctatctatctatctatctatctatctatctatctatacagtcagccctctgtatccatgggttgtacatccatagattgaaccaatcttggatagaaaatattcaggaaaaaattgtattgtaaacatacagactatttttctttttattatttcctaaataatacagtataacaactatttacatcatatttacattgtactaggtataaataatctggggatgatttaaataatacaggaggatgtacataggttatatgcaaatactacaccattttatatgaaggacctaagcatcctcagattttggtatccacatatgcaaatactacaccattttatatgaagtacctaagcatcctcagattttggtatccacaggaggtcctgcaaccaatcccctgagtataccaagggatgactgtatgtataaacgtaatgccccaatgggtatgttattttaggaagaaggccactgagacattcatctactgttgggaaaattccatcttcagtgctcaaatatgaaccagaccaggaactgatgcagattattactgctcatctgacaacagaggttctaatttttcattccttcagtgttgaaacaatctctactgaaccagcttcaaacaagttcactggagtttgtttcaatattgcaagaatgataagatggaagctactttcatcagcatgaattataactactcgtatacagattgtcgtatttccactgttactgatgttaaaactgcattaaagcaaataaaaatataattgaaaatacaagcatagggttttctacttgttatcctcggacatactatttgcctatggtttccatgaatttccaactagagcaagaaaatctcttggttggttgtcagccatataaaaagagaattcagaatgttacttttgggaagaaagccagatttaaacaaaatacttcccagtgtttagcatatctaatacaagtgacagataagatttatatatgacatcatgttccatcttctaattcgatttgaagatggtgagaaaatggtccagaaattaataatacatcaaatgatcattctcatattaactgaagattttagagccacaaaagaagaatctaaaataaatgtataagttgaaccaataattaatatctctattgtgaaggcatcatgatccattcttgttttgatggttgcctaatatgtaattgaatttttaaaacacacatattcatacaaaagttgagtaataaactaaaaaaagaaacctcaacatggaatcccttcaaattactcttttttaaaaattttgcaaatcgtgttccaattccattaggaatatctaagaattttatgactgatgattctttattgttcataatttcaagtatcagaaatatattttatagtgttggtccatcttgccagactgtaagctctatgagagcacagtctacacttattttattcagcactgtatcctcagcatctggcacaatgttgggctcatcaagtttgatcaatatttgctgactgatctgaagaccagtggggaaagtgtcatatatcaaaagaattgtgttcagctacactggtaaaggatttgcacagttataatcatttttatgggttcattactttctcttctgacataaattcaagagacattaaaattagaaatcaacaaaatgtttaaagagtagtgtcaaccttattcacaattggtgtccatttgaagttatcacagtaatgttataataatgataagtattcaccctcttctctttgcaagatatttccactaggatggcttgctaccatctccaaatctaacccaaaataggacttacttaccaagggggagaagttccccctaataaggttttcctttcccagcatcttccctttcttgggaactatttttttccccaaatatctaggctaattatctgggctatttgactattactgtttcccattccttgtattcaaatatgccttcatgtttttattattgagaaggtgctaagagtggtactttcttgttccaacatggactgatctagtccagccaggccttcaacttcctggctgcccccaccccaccccgcaactttaacctgtggacatctgatgcctgctgcatgaattttactaaaaattctcctttcacaaccttcactgatcaaaaatctacaaaacatctttatcatatatattatgttatataccatgttttaacaatattagctaaaatgttttgaacctttcatgtccctgacacttggtgtttattgtcttgttcatcctcacaacatgacagatgataaatattattcttaccgctcttcttattttacgtttcctaggttgaggaaacagatctagtgagaggttgctaacctgctcaaagtgacaaaacaagttttaactctttttttgttatctgaagattcagataatgcagtcttgaaagcaaagtcctgaccttcttaccctcaaatatgctccccttcaggcaagatggttttctcacagtggctcatgcatgtcatttccaacctccagacttttgtgggaaaggtccctccatcccaaaaggcttctcgacccccccttgtgcctatgcagaaacttcccacactcaagatacaattcaaattctaccttgtatctgtagccatcctgaatatttttgctccctactctctccaaactctgaattttgattggattctgtgctggacagcttagcagttagtgatgtgctgtcgtgtaggacatatacaatgacacacatggaaaacagtgaggattcaccatgaagagccaggaatatacatgtcaacttaatagggatgagttttgtttagaaactagcacatatagaagagtacataacaccacttctgccatcaatacaattggagaaacttcaatcttagaattgaaatttcaacgggtatgatagaaattaagcagcattcctaaatttagaaaaaggaaagtcagtgcaggctgatgtttttaggtctcctcaaaaaaagaaaaaaaaatctctatgcagaacatggatattaaaaattcgcagattccaacaccactaaaaacattggtcatagccacaaaagaaattgaaatctttataaacacaagttaggaagaacagtgtgacagagtgatcttttcaaacacccacattacctttgaaaaagagaggcttgtgatggtgatgattaactgaaactgaaaaaaactattgtcaatctgtaccaaaatgcaaatgtgctgagataaatatctaagaaatacattgatttttcattagtgaacatgaattgccctgtacaggtcatctgctgttgaaaccaatttaaggagattatggtcaattatgaggagcagaaaacatattttgtttgttccatggaatagatgggataaatttctggcagtgtacacagcaccaataaaatttagctgaaagaatcaaataaaaaagcatatttattgaactatgtctattttttaataacatgattcatgattcttaagagtaaggcaaagttgatttcatatttgtattttttggaaattagattatttgttttgtttacttctaaactttagaatcataatacagttaaaacaatcctagctgataaagaggtatatgagtgtgtaggtgttatccattttcaatagtaaatggaaactaatgcaaatgtaaaatgtgccagtagaatttgtagatgaaatgtgaaaacattttttgtaatatatatgaaaaaaactgtacatagagaccatgaataagtattgcttagagagcaaaaataaatgttttcaaactttttcattgtctcatttatttgtgctcacccattcctcctgtctataagtaactgatttttctttttaatacagcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtaaagagaaggatatatatgaacatatgtttgaaacaccattacagacattttcttttgtggttaaatatgtgttgaataattttaatttaatatgccagcagttttcacagcagttttcacactatggtatgtttgtgtatttcatgcagtgcatatgcctaatttctttgcccaagttcatctgactgcaatttggctaacatctgccctcatgataaaattaccgcaataattcagtattcacacctggtgagccggaacagtaaaggaccgagtgagggaggggatgtacaaaatgacaggaaagggaattggaaaatagagggcagagaatttgcctgaggtggaccctatcaaaccctatcttgcttggattaattaggtagtatcttccctttaactgtcaattggttggcaacccatactattcttccacactttctggccccactttccaggagaattacaaaaaggtacctcctagccagacattgtttatctaccagcttctctagaatggctgtatgtgactgcccagatattttcaggtctaaacatattctattagctgtagaggacccatagaaaaacaaaaggggagatgtgatgggcaagctcattgcaacatccctttaaattctcctacttgtcccctgaggcatttgaaaagattcaataagatcttttactatatatatatatatatatatatatttaagacagggtctcactctctcgcccaggacgatcctcccactttagcctcctgagcagttgggactacaggtgcatgccaccatgcccagcaaatttttgtatttttttgtagagatggggttttgccatgttgtccatgctggtctccaactcctgggctcaggagaggttcccaaagtgctaggattacaggcgtgagacactgcatctggcctactaccatgtacattttaagacaacccaacttggtagggaaaaaaatggtaaaatatactctgttactgcacattctttgctttcaaatagttcaagttttagtttgtagttcttatttttaccattttcctttaaacaaaaacatcaagtcaactgaaaggcagatacatattattcttctgtctgcagtaatttcagtgtttatcaagaactgaacaggcattagacataactctgaccaaaggagaaacaatttcgttgaagccagtgagacaataaaaacacagaactgatatttttggggttttctgagagctcttttcattttttaaataatttttgagagctttcttgaaagcctggatctgattcttttgaatacatacctttatagaagtaaatcttcatggttttaggtagaattttttttttttttttttttttttttttttttttgagagagggtcttgcagtctttggggcatttgcttttttgatgcactcaactttggaaccttttacaaagtagaaatagaaccctgcttgtcatccaatgagataatttggtcacagatatgctaagcctatttgaaactttatttccaaaataaatgacttggcttgtttgaaatgtggttggttatgactggtgtagtgagctcagctacttgtaatatcgtcttgggatcaatcttctcatttagttaaatccacaaggcttcaatatcaatttccttctaaatggccaggtccatattgcaatcgcacctgggatggatggctgtgctgggatgacacaccggctggagtattgtcctatcagttctgcccagattattttccggattttgatccatcaggtagagtgtttattttcattttatgttcttgctatgaagcttttagagttttcatggttttgagaagaaaatcagatacaaaatgtacccacctaatcatactccacattttagcaacttctgaagtgtttccttaattgaaatacttcttagttgtacaaatattacaagctgtccttttttttaagtcatgttgctggtcagttatatgtgttggtattaaattctggtttataaaaatagcttttctaaaactctgtgaatagttttatttaaatgtgagttttaaaaatttttcaattgtctcactgaaaaaggggtagaaatcttaattatgaaggagacccagatgctgaaactaaatggattagctatgggcacaacatataggtccctgttaatgggtgaaatttgttcccaagagtaaagtgagcctgagcctacaggggttaagtggctctagaatcccaattaaagaagaaaaatgagtctacccctgaaacaatgttagaaaaatgctttttgtgcttccttccagagtgaaagatctaatctgtccctgaaaacaaaatttcacctcagtaaggcagagataatggaagtggaattgcagaggacctcttttaaaaccagtgcaggccagccacttctgggaattcccattgtgatggaaaagttgggatgtgaagaaagaaagggctttgtgctgtgccctaaatcagctacattcctggaatctctaactaaatgctatttgatgataatccttcaggcccttattgactcttcccaatgaatgatgcttcattaatcttgatttgacaactacattagaagttacttgattggcacgaagagctgttgtgcaattgaggctactaggccagttccaaagtaacaatggggctcttgccaaagtggatataaatagtgactacaaggcaaggttttgataaaaaaatcagagatttacaggcatcgatccatcttttcagcctggttcttatacagaaacctctcagctgcaggataaattcattttaaaagtgtatctgaaatatatacatatattccaaacttaaattataaacatatacataacataatatatatatacacatacacacacataccaaacttaaattcttcaggattttcttattgacctaaagcttacatcttaatgatgtacaaaacacattaatgaaatgccagaggtttgtttcactttggaaaaagtacttggtaaaagatagtgaaagagtgaagaactgtattgaaattcaaaagaggctgacattctgaaggaatatgctggaatgtaaaaagcaaaatttaacaaggaagcatttacagccctgcacttgaagacaaacaaacaattttaatggcacagagccattaaatggtccaaatatggtttgggacggtgattctcaacccagatgccctttcacaaaatgaaaaaaaaaattgtgagcttatgaaaaatgcagatgcctgacccctgtccagatcaatagaatttgaatctctagggacaggacctcggaatctgcaagtagttcccctgcgtgaatctggagtgagagaagcacattgtacttttcatgggtcagtccacacatagagtattttagagagaaatatcaattatcaaagcacatgtggagagtctccaggctgttagcggaaaatctgtggggagtgaataatgggggtgtttggtttctaaagggaagtcccagagtaaacataatcctttcttcaagtattgaaagactcatcaggtgaagtaggacaaattcttgatagttataacagttaaaaccaagaaatgactcgatccagactttagttaaaacaaaacaaaaaataccatttgctatggccttgttcatgagtggaatgaccttctttaagagtagtatctctctgcatacaatatattttcccatacctggctgcataacttcattgaaaccaaattacattcattttatttaaaaaaaaaactactttctgcaatctaataaatataaattgtggccacttttaaaaattgcaagtaagcttggaagggttgtcattgacaaataacattagcaaaataatcgacaaattttcaacatcaattttttgtgctgattagtgctaatcctttttcacatgttgttaatgtccaatagagatgctttaaatagggtttgtctagtggctatctcagtaaatttacagttttaagttttcttatttaactttgtaacagatttacatgaagcagtaatgattctaataggttgcttgaaaacatcttttgcttgaaaaaatgttttttcatccattttaaataaaaattaaacatacaaataactctataacctgcagtttaaatcttgcatttagttcaataagacctgttttggtcatacaataaaaaatgatgaattatacctaaaaattgccaagttgaaaggatttttttactagctcttagcagattgagtaaccaaaactactaatagaaaattattttaaatattatcagatctcagtgatttttcagcccaataagtttctcttttctttagtatgttatttccttagccttttaaacaatagcagtgttcttcaaatattaccatatgattctgatttccacatcctgtgctaaaaaagaacatagccatctataaaagcattatctatacgtacaacaatgctttgcctttgttaacatagaaaaatgcttcaaaaatattttccctgagaaatgatcaaagattaaaattttgtgaagattccaaatacacttgcaaaatctccctgcatattataccaatctaggcttgctatttgatctctcagacaataaacacatcgaatgggtgctttgaagaactggagagtatctgaataactcagattaaatttggcaagtacattcattttagtgtttacttgttcattatttctatgttatttaattcaacctgattttttctttcagttgcttggccaattcagaggttagaatttaaatattatcttgtagctaacaacattatctaaaattctttaggtcactggaatggaatgtagataaccttgatcatttatgagatttcaccttctagagtcccagtaccaaattaaaatattgttgaccagtattataatacaaaagcgaggttttttttagacaatccagccaaattctaattttatttcagtgccattttagttgtttgctaacaattatactagttttacagtgtgtttatcctgctgataatagtgtcttgataaatattatctcaccattctgaccactttttactcccaatatatattgatgtgatttcccagcctcatgcactttacccagaatctcagatatatactcagttaataattaagcaaaatacagaacaaagaagttttcttatttatttttaagatggagcacttgatgtagggtctggtatattttaagttatcaaatgttattttaaaaaattattgtagttgctatggtggcctaatattttagtgtgaatttcagcaaagatatgagtcatccccatgagaacttattatttctatcaagtttggaactttactttgtactgatacatatacactgcctaaaaaaattaaaataaaaaaagcagcaaggctttatcatactgtaaccagcactgaatatatcacacaaaaataattgaaaaaataataagcctgatgggaaccagcagacaccacaaagcaagcacttggaaaaagttctttctcagggctcaaagaaaatcttcttatccttttcctccaaaatattttagtgtaagctgaactcttaagctcagtgggcagccccagaatcactgacctgctggttggagggatagtaagatgtcaggctttcctcctctttgctaagcatcaatttcaaaaaaaaaaaaaaaaaggggggggggccaaaccagtgttaccttttatttctatgccaaatccaacttccaggtaccatttacatcatcttattttctcacctacatctgcatcttaacaataagggcttgcctgttttgtttcctctgaccagttggcacattgccacagaaataagaggcttaattagtgcttaatagctctgcaatagatttggaatcaagtagactcggatttgattcctgacctttctacctgctagttatggaagggaaatttacttatatataaaatgggaagaacaatattcacttcaaagactcattattaacgttaaagaagataattctgcaaagtgctgagcacagtgcctggcacccccatagcgaaggtggagatctttaatatgccaatccagctgagttagggatttcattaactagcatctatgagatactttaaaaagtcaacattaaaggctgaagcctggatccctatatattttctgtgggatgaagagagatgattcaaatgaaatctcttgagaggcagaaaaatggcctcttgatatatcccttagttttggacttctttgttctattggatttaaccatatgggattgggaagatttgaacatttgatccaaaaattactgtttcatacagttaatctaatgaaaaaacattgcaagtaaataaagaaaggtatgttcttgttcttctctagtagtttccatctataatcaaaagtttaatgtctgttaaaaaattatgaggtagagtataggtagcactttggagtctgaccgatatccatgtctttgggtctaaatcctggttcaatctcaagctccatgactcctctcactgtctgctttgaatttgtaaaatgaagatggcaatgcctacttcacatggctttgctaaagataaaatgagacaatacatattaatacactttgtataatacatgatttgtgatagtctctactgacatactcacattcaagttctataaagtgattaatatgttggaatggcttttaagctttaatttattatttttccttgttgggattttgagatattaatgcaataactgttatgtttcattaacagaaaaggttacaaaatactgtgatgaaaaaggtgtttggtttaaacatcctgaaaacaatcgaacctggtccaactatactatgtgcaatgctttcactcctgagaaactgaaggtaggttctttttcacgtttcagtattgagtgagaatgtcattatttgtacaaggaaatggacgcataactgtaaaatatctgccacaaagaagccaacaggttttgtggaaagaaatcatatgataactggtaagttctaggaatctctagagtagaattagagaaaatgacatttcacttcttggttggcactcagagtcaagagtcaattgaagttccgtgagcatacagaatcagtccatttatggcagggggataagtgtccatagagcagatgaaaggattcccaatgatgccagaaattaggcaacatttcaagggactctctaacatctcttttcctggaagagtttcaaaactacagatttttccacaagtcacaagatagcatgcttcccctgtgttagccacaaccttagttttgtaataaagcatagaaagaactcctaaagtagggcatctcatcagtccactaatacagatatggctgcttagattattttcagtggaagttcattctcaattgttagtacttgcattttactgtatgttaaatattttgaatattatctctgcttagaaacatttatctaagcaaatggcgagtggggaacagatgatctttcttcctcaaatgtatcaaaaatctgaaaaattcaccaagtctacaaccagcctcctcaccctacagaacaaggagttacagttggtcaatgtttagctgtgcagggcgtctctaagcaaccctgctgactctaacataaacctattatttctgtcaaagataggcctgtatactaaataattattcagttgaaaactaaaagcacattagtgtctttgtttgctgttgagatgctttcagggtgtagaaaagttttcttttattaaaaacaaaacaaataacaaaaacaaacgagatgaacatatggcttagggaccacttgaataggagggacacctcatcacaagcacaagctaagggcaccacagccagttatctctttacaaatggtttagtcaccctgagaatcagatgcatgtctacaccctaatggagagctgttaataaagtctgattaataagctatgtcacagagtagtgaattttccgaatgagtgttgattatgatgttacagagaaaaattatactcatgttaaccagattgttgtaagtagtgcaagtccaaatcattcttagtgttgtttttggacttctcacgtacactggccacatctaagaatgaaataatttaaagaagaaatgctactctctaggcacttagtataatgattaaaatgcttttcacattctcagtaaactatcgcaagaacaaaaaaccaaacaccgcatattctcactcataggtgggaattgaacaatgagaacacatggacacaggaaggggaacatcacactctggggactgttgtggggtggggggaggggtgagggatagcattgggagatatacctaatgctagatgacaagttagtgggtgcagcgcaccagcatgtcacatgtgtacatatgtaactaacctgcacattgtgcacatgtaccctaaaacttaaagtataataattaacaataaaataaaataaaatattgaaaaaataaaaataaaaataaaaaaataaaatgctttccacaaatcttccatataaatatagtcacattaaaatttgaaggaaccaacctagaattcattaaaaaaaaagtcaaccttgagaatgacacaaggatggcaactaaattatgagttgttgatttaatcttacacatttagacaactctatttcagaagaacaaatgtttttaaaaggagtactctggaagcaactgaaaagaatatgaattaagttatataaatttttcttagaaataaagtatgcaaagattttttaaaagaccgacagaagagggggtgcagatacatatccaaattaattaaggggcaagagtgttctacatagtaaactatttagaaggaagaacttttagaaaatgctcctcctgatattgtctacaagtgaagttagaaaaatagccagaaaatgaattcctgggagaaaagataggtgagtgtgtgtgtgtgtgtgtgtgtgtgtataagattcctaaatccctcccaagaaagctacctgaagattttatcagctccaattataaaccaactattctaactttattaacatttcctgtaagagtaaaatgtgctattaagagagtggtatcaacaatgaaaatgccccaaaaatatggaatgcctggtcaaatgatgcaacaggcacaacttaggagaagctaattagtccattttgaaattctaaggcctagaaaaggaatgttcatttcaggataacaataatgcacataaccaagaaagggctctgaatagtttggatgcattttagctgactagggcttcaatcccagactctttctgaggaaattgacaagaatcaaaatgttcaaggatatataaattctacatttattccaggaattcaaagaacttcatagaaggtattcatcctatctacactacatttgcattcataccaagacccaacaaactgttgaaggaacttaagaccaaaagccaagagattaaagtacaactacaaaataatacacaaattatgcttcaccaagaaaaacaagtgaaaacaaaatccctagttgcaaaagattcaagtctacttcaattacagctgatgaaattggctaaatagtacaaaatgcaaaagctagtgtcaaaatagagtgaaagagagagaaagagagagagagagaccagaaacacagaaacagataaagagaaacagagagacagagaagacagagagagggatgggtgactggatcaatctctattttctctgttgataaaattacattataaggaaaggaaagactgtgattactaggaaaaaagatgctatgacattatgcattttatgagaaggacattcttccgagtcaacataacattatgggagaataaataaaattaaccatgatcgtgaaaaaaaaaaagaattatcaaatggctgccaaggggaaaggtttcggcttcaaccaactaaatactaatgatatcattttaaacaaaggcacagaaacctgccaccatactctctggaccctctgtaggtcaggacagcttggaaagtcgattactgctttgccagttctgtggtatagaattatgtatccactgtctaaaagattccagaagttaaaaaactaccaggattgcttactgggcttagggatgataatttttaatgcatttaaagctacttctgaaatatatttttgaatgaggtacttcatctcaaggaactttattctttgaaaatgacaatttattttcataaaatgacatgagtaaagctaaaaattaatttcactatctgaaatatttgttataattgtagtaggtttttaaaaggaggaaaacatttgttgatttacatatatttagaaatagaaatgattttctctctgatgaatcattcattcattgtttgtttgtttactttttgttgttccagaatgcatatgttctgtactatttggctattgtgggtcattctttgtcaattttcaccctagtgatttccctggggattttcgtgtttttcaggtaagtacactcacagcatctgctctttatttcctcctttaagttactgaaacgaatggtgaattctgcaagagtggaggtggggaagggtcttcagcaaattacaagcgacatgcaacattaggagtcatgaagtagcaagaaaaatgaaatgaatttaaatatgcttttaatcctatctttggggagaacaggataaatttatatcattttcaaaagtactatatcttgagacttcccaaaataagacagtcctgtgaatccataagtatactgttgtaatatggtaaaggttccatttaccatgggtaagttaccattaagggtggtggagaggattctccaccttcttttactcccactctaatttttggagaccatagccacaaatccatatataccctgcctaattatttcttgctagcatatgtaagactaaggaataaacaagcacatcagagcatgtttctacaaaacctaatttaaataatgcttgtctttaggttttatggtgcacaattatgcttgaaagcaaaatatgagaaaacatacaaggaaaacttgaatactttaaattatcttgtcatcttacatgaatataaaataggatctttacaggaatggtacatttaagttagaaactgttacatatgctttcatcacaaccaactttagtgaaaataaagcactcataacattgtcctaagctggatttctcaagtatgttcacaaatgtagattcagagtaaactgtatgtgtattttttttttagacacaccacggttcagtaaaaatgttagaaactgaagtaaattaggtaaaaatgaaataatacatgtaaggtgcctggattcatgtctgacacataataagtgaaatatgtgtttgatatcattattattattattattggtagtaataatagtcatggtggtatcacagaaatgataaagaaaagcccttttgtagactcagatgaccaacaatcatccacacttaaaaaatgtggtatgtgattaaatgtacagtgattcacatatagaatgaggggggagaagaatatatatgcatttatcactactgaactatatgtttaaaatggtaaagatggtacattatacatctatactttaccgcaaaaaataaatttaaaacatagtagaatcttaatttgatttttgttaaaatattgtcaggatagtactaaggaaaattaaaccaaggggagaataatcacctgtaaatacatccttagtacaagtactatttttttccttcatcatttttcaatctttgtctatatcttagtcaagattcttatattgactaagcaatagaggtgaacagcaatttttaactggatagcaaatgataacataagcaaacagacaatgagctccttccataaatggtttaacaggagaaagactgataaagcaaaaagacatgaagtattaattactcaaattagaatagaagagtattttcaatgataatgatcagaatacagtaatcacataacatgattaatcttggaatgtcttcatttttcaattgcacttcattatggtaaataaacacatcatcatggttctcaaacaacttttattttaaaatatgccactaaagcatattttttaagttttttaaatataatcataaaaaaattgaaagaaaaattccccaaatttgtaatcatgcaactaattgtagtcaggactttaaaggtaataatgtcaaaggcatagatcttaattctttatttctccagtcacttaaataagtgaggagatgtgagagccaaatcagcaaagaaattaagaatttttttttcaacgatcatttctatattaagctactgaagatgggtgtgttaaagtgtggcacaagggcaaagtagaatatttctcaactccagttagtcccaaagcaaaatgattctgatcaaacccttgagagagtgtgggggcagattgtctagaaagtacccaactttgccctaccgtctctcagttctaagaaccaactaaacagcaacttaaatctggggaagaaaaacaagtcttattgatttatcatctgagcctaataatgagaagaaatgaactgaaatagctggttcttcagagcagacacaacaagagacaaatgagatatgaaaagcacatatgctgagatgccaaatattcaaaccaaaaaaagctgtataaaatttttatacttgttttcaatcatatcttaaaccttacccaaactaaaaatataaagggcatctgtcttcttaaaatttaagtatgatcctaaccctttaatgttttttatatttgaatttcagaaacattaaaagtgattgtggaagtagaaaataatactgatctatcattggaatttatttgtgaattagcaaattttttccagaattttttttcaaatactctacatcaccatcacctttaagatctttactgcacattacaataggtttcataatttatgcacttaaatgggatcatctgtgtgcaacctgagcaaaaaatatcatcactcccttacttaattctatattcgcagggttgagtaattttgcaaactccaaacaattttataaaactttcaggaaaccagagtgaaaagttacccagctccacttctgagagctgacgccccattcacaatcattccattgaaatctcactgtaaagccctcagaactcctcaaatgtgtctcataaaaaggaggaaccctgcagctgtcctcctcaaatgagcaggcctcctaggaggcctgcagagaaatttaaagtcactgatcatacaagaggctttggaactgccaatacttcactaaaaacttgggatattactcagaattagtttattaaagcatgcctttaatcttaatgtaattttattctgagcatgcaggggaaggtaaaaggtggaattatgtaaacttaagcaaatagaaatcatgctccttgtgctgttggaaaattaatgtataaatgtacataaagtcttcagtttaatagcagaaaatttatcttctatgcattcttcctagagaaagacaaaatgtagaaaaaacaagatttttcaggtttaattcatggaaaagtcagcattaacaatcttaactgttcaaaagtttgaataatagtcaagatgatatgcaggtcatttctggtacaccagttaaagatagaagacagcattattaggatggtatttagtttaaaaccttcgcagaaaaaattggaatgatattatggaaagggattaaatataaacatcaaattagttcagcaagatttcttactgctagattccaatagtaagtgtcaacaagagagctaaaatgaagatacccttagggaagactggtttgggaaagacacataaatggacatttatttcagtttgattcttgaccattttcagcaaatttccaataaatttacttatccataaatttcaattacaatttatcaatttacttttctgtggaataatcctaaatatgtaagcctctcattgtatcacttcatgaaaagcataacagaccattcattagcatttattttgactcattgttatccatgtctttctgattggtattttaaatattattcttgcctgaaagctgatagaaatataggttggaaaacagatggtctcttcattttattgtctacattacgttacattggccacattgacaaaaagcatatataggtaatcatggaggtcaacaaaagtggcttccagtggaacatactgagggaagactgattgctctactacttactattctctttgttctacgtcatctctacatattgggtgggtatgtgaatctgtaccttgtgtagtatccaccattatacatctctagctgccttcctgttgctccaaggaaaccatgaaatcaatggtatcaaagatggcctcctttcttccaactcaccactggcaggacagacaccaaagataactgttgatcataccacggcatattcttcacccatagaacagtagtaataggaaaagcaagaattttgattgaaagagaatttattaatgataaggattttgtgctaaatcaaaaggcataaagtctcttgtcaaatagggaagatatataatatattgtatttgaattttcccaggagttttaatgcaatccattttacttcattttctctctttttttcagttctcatgtttatcatgaataacacgtgatcctaggtaaatgaaaactaaaagttacactagaagatatgacttgggcttgtaccaattacagaaatgattcctaattcatattttcccatgcaccagtaacttcaaaacaacttttacttgttttctttttgctactttgtttgctttgaatgtgactataattacactgtcttacataggagatatacaggagaaaatactgctgtttctcaccctgtgatagcaaactgatttagtcaactggcaaaactcaagaccaatatcaaatgccgtctcatacaactacaagctcccaaaaagtacacactcacacacacgtagggtactaataacttgaaacaaatgtgaaagatttacaccagatgaactataaccatgaaaatgactttttgctttagccttaaaatgtgacatcttgagattcatactcaagtaaatcattatctctttctcacatttgaatgacaccttgattggtagattatgttgtagaagtaaattatgttgtctcatcctcctactactggagaatttatggtacttatggtttcccatcactcaaataataagcaatgttctgtccatctaaatgtctgtttttttcataaagggaatagaatatagccagtgtcaagttagtgctatttctacctaagacatctcagatctgacaaaaaaatgttgtcggcttctctcatgttcatttaagcgacgtatttgtactgaggatttttaatgttaatttcaattaattctctctagcatcccacacctcttcttcagaacaattatcacagtaaaattaaattaaaaatggcatagtatataagggccctgaaggcaagagcgcctgtttgtcctctgttatattcctgatatctaccaggtctggatcataatgagtgcacaatcatatattttggattattgaatgaattttgaaaaatttgcaataccttcgactcacttcaaaaagatgactgtaaatgtcatcaagttagtataatacaaattttaaaactcaatgagcaaaaattatattaaaactttcaaaaaacagaaactcatatattagtcatacttctcctttggaggctagcagtttatctagttttaaactatcattttctctgtgtttccaatgaaaaatatatatatttttatatagagagatacagtactctttatatgtactgtatctatctaaatatatatatactgtttatatgcatatatgctgtatgatatattcatttttaactagtgttgaacaacaggataatgttccatgttagctagttaatacgaaactgaagaatttggcaaatgttcagtatgatatgaagtataaagtaggaaatcatctagcatttaatgtaattctttggtgtactggaaaattgcttagtggtcacagctcaaaacagaacatttttcttaaatacatttttaaatttaaaaaaaaatgggtattaagcccaatgatgtgtcagagagtgttctagatgtataaaataaattcctttagcactttcattttagtgggaggaaaggagagaataacaggcaataaataaatatatactgtgtcagcaggtgataagtgttaagaagaaaagtagagcaggcacacaaaagagtaagctggaggtagaggaagtatattttattcagggtggcttgggtttacacaagagaatgaagttaatgaagtcatttaagctaatgtacaacagcagattattgaacactccccaatcaaaagaaaaccaggcttaagaatatcattgctgtatgatacattcattcatactccttagttctagataatgtcaattatcattggtctaagccaggctgaattaaacaatgaatcccataattaactgctagtgagaattataaattgctgttgtgactatgaaggatggcaggtcacagattcaactgtgctgattgcagcagtcaccagatggaagtttattcagtataaacgagcatgaggtttcacataagtaatttttttgtttaaatttaaatttgcaacctacctttagtgctattataaaaccagttgggtttgatttatttaacagacttctgtactgctgaatagagtagtccacagaagcgtgtgtgtaagagaaagctcttctctaaatcctgtctttatcagagcctgcctcatggatgtaaattctacatcctatacctgtttaacaagttaaatttttaaaaatattatttattaatgagttactataatcgcttcccgaaaagttgaaaactgtaactataatagttaaaatatgctctaacagctgcctttattacttaaattgctttttattgcagaaaattgacaactatttttcctttgaattggaaatataggaaggcattgtaagtaaacccaggctatttttgtatttcatctttccccagaatttccttttcaaaattcttcatttctaactttattccatgaagatatttttaggaatagttagtgttatgagtcattggcatgttttacgctctcacatggtgaatgacattatactaatattttttgccttatcaaatatgataaatataagattttcatttgtctagaataatcaccgcaattttgttatagccatgatgtaggctctctctattgcatcaaaaaatgaaataggatattcaaattaatgtttatttatgtgaacattcacaccaacacatatgtacaactttatgaactggagagaaaagaataaaagccccaataaattgcttaatacacaaaaggtggggaatgactctaccaagaagcagcagcacttggatagctctcattggatccaaatcacagtatgcctcataatgagatttcatttgagcctattaagaattctctgcctgtgtctgagctccaccatcaggagggttggaaaagaggaactacagtgatgattctagttgaaatataagatctaagaagatggtaacagaaatgatggaagaggaggatgccaggagacgtgaagtgtgtgtatgcaatggctcaaggacatagattcagtaccttgtcacaggcagccttggttctgacttcacgctctgtgacctcactggccacatattttcttcctggttgcaacaatgaacagtaggcttttcatttgaagcacactgcagaccctaatagttgctaatctaagagtccttctgttaactaattttacaacatttgttataattaaaagagattttatcacccactctttcagttattttatgaagtcaccctgaggactgaaaactatgacctgctatgtttggaaacatgttttttaaaatcaggcagaaaagtaggaataaatctgaaagaggagaccagccttactttcttctatatgttctttcatcttttttttaaagaaaatatgaaaaatattaccaaaatgtttttttccaggtactccaacattttggcactggttaatgctttatgttactttttcttccaggagccttggctgccaaagggtaaccctgcacaagaacatgtttcttacttacattctgaattctatgattatcatcatccacctggttgaagtagtacccaatggagagctcgtgcgaagggacccggtaagtactgcatagttttgtttttacttttattttaaaggatatagtacctgtaaatagtgaacatggtgttcatgttggacttaagcagctgtttacatgtgtaatcatatttacttctaccgtcgagttttgcattttgtgaggcatatcatttctatgtagtttctgtaagaaattatagcagataagaatcatacaacaaaccatatatatatatatatatacatatatatatatatatatatttttttttttttttttttagatagagtctcactctgttgcccaggctggagtgcagtggtgggatctcagctcactgcaacctccgcctcccgggttcaagcaattctcctgcctcagcctcctgagtagctgggattacaggcatgcgacaccatgcccggctaatttttttgtatttttagtagaaacggggtttcaccatattggccaggctgatcttgaactcctgacttcgtgatctgcctgcctcggcctcccaaaatgctgggattacaggtgtgagccaccacacccggcccaaaccatgcatttctaagactaaccagtaatttattaacatttatgacaaatgtattgagcacctcttgtgaacatagatgatactaggtacactaaggcagtgcttccccgtgattctagtgtgccacaaatggttacaggtgtgcctagatattgatcccctcttcattctgttgccaagttgggcttggcccagtcatctctcctatgagtagctattcccttttgctaatgaaccatataaatatgatcatgttttgtgtcattatgctttggaaaaggtttataatgacacaggaaatgcaaaagttagggcaacaggacatacatctctgaaactcatgcccacatgcacaattgcaacaagcaaatatggtgactgtcctccttctatttgagtactatagagaacaggagagctgaaatcatttagctccattactgagaaccattctggatttttcctaccagaattgtcctgagagttgttcagaataacccatatttgtcatcagtcttctctctttatactcaagatactgtttttcacagtgatttccaatcctggctgcacttcagaatcaacaggggagatttcataaatatgaaatagctactgtccacatggtactagagagtcagtaggtatgaggtggagccgaggaatataccctggatatttcttctgtgctgccaagtttggaaatcatcagccaactactggatgaagcaaataaactggcaggggaggacagaggaggagcaccttgaggctctgctttttcattgatatcagtccctggtattacaaactaacaaaaaccagaactaaaaaggattttggaggttatttatccctgactcctcattttacagatcagaaacttgaggataaaaaggcaaagcaatttgcctcaaatcatataactgattggagcggggagcaggcactcaaattcaggtctccttactctcagtttttgttctttcctttgcattgtcacatgtgtctctgtaaatcagtgagggcttagtaacaccaaactggttgattagttttccacatctaccagattaattttttaaaaatcaaatcactttggtgctgtgataccaagtaaccatttactttacagaaatttttatttatcaatccactgtttaaataatttataattgcagcctctattatttaatagttattttgccctcagtaaatattttcatcccaaaaagtccccaagtacttctctgctttctcctttgcaaacctgctttccttaattttaacaacaaatcttttccttatttgttaatggactgcactttaattgaatgattcaaaatagtttaatgatataactgtttattagattgagtatgaaattaggaatatttgataatttttgttctgcaaaattggtgaatccatatgttttatcctaatgcaaatgtggagacaatacgctggaatctagtaacagtagggactattatcaccccaggcctgaatggacaagtggagggaaaggtatatgaacccgaagacagagaggtggtgtggtaaaggaggccctgcggaaactgaagacttctgtcttaggacacggccagcctatggcaaccccacaaaaggaagtggagaaataaatatcccttgttcactcactctcatattccagtctctactgatggtccccatagctgagctcaaccagaaaccagagggcaaggcagccctgctggagatgtacagatttatttctgtgggcccagggcagtgggagcaggatggaaagtgcatctggaagcacaaatagaagctatctagctcacagtgaagggccaagggatattttcatttttgtttctctgcttaactgtgttatgcaaacatctgtagatagcctcatctttcaaaactggcttttgacaattctgccaacccaataactaaaatgcgtgaaatctgcccagagcatgcatcctgtgtgacagtggcgtggcgtgggaaagcagcttaatttggcttctaatggctccagagtgttaataatttttgtggtatgaatatatgcagagagacattataatctatcttttaaaaccatggcatctgttatcagagacaaagtccagtcttacctgcagaataatgggaccaagtgtgtttatcttgatgaattttcctttttaatgtcaattgatttaaagaaaatttatctctaatttaatctttgcaatatccgagcaatggatggaattatccccatttttaaagctggggaaaccgaggctcggtgagagtaagtgtcctccgtagcatgagtacttagagatgggattcaaatccagacctatctcacacagcagccaacctctcttctttaccacttggtgctgttgattgtctttatttattgtcctagaaagatgtgttcttaaagaaatgggctcagccattccgaagtgatcgccgcctataaccagacatgcccactacagttaattagggaagctgaaccccactgaggtgaaaacagaggtagaagcaattaactaagaatctctctgcagaaaacagtcacattttggctctccatttcccattaagagtagaaaccgtaggaaagttactctctgtacggagcaggccaagaaatgaggcttccgtttttgaataatcaactacattgatctgggagcatgctcagtcacattacgaagtatcataattcttgaatagactctttctcttctttttttgggagtctcacacccatatctccctgtctcccatctgtaaatgtaaaagggacttcttatccactgtgagatcttgttcaacctatcttcctgaaatagtcaaaattcaggtttttttctgagtttgctactaaattatagaaaccatgtagctttatgcatctaatgagaataacaaaaactgctctgctcacttcccagcggttgtcctggagcccaaagaagcataaagcttttcaaaactcccaagggcctaatattacaagtgattgctcacaaggttgctgtcaactctcaccttatgactaactccctaggtctctgtacttgacttttccaacaaatctgttactctattaattgctcttggcagtctcattattgtcattgctagatgtttccactttcgtaggctttttaggggtagacatgcccagggactctgccagacacagcacataggctgtcccccagccaccactaagtcaaaacacatatgccctgcagctcctgtatcacctagtaacctttaataaaccccataagcaggtgtctagggacctggctctgccactaggcacctgatttcctaggagaatatgagtgtgagatttagggagccagagaaattaacaggaaactttcttctgtgtatagaggagtgagattgattcacccaaacacactaggcattcttaaagtatgtttgaaagaaaggaaagaagcttatctttatgacatgactagcacctggttcttccaatgtatttttaacctaacagacttgctggttttagctatatgagagcaaacttcctaaggaaaaacaataaaacattttaaagatagaattttatcccatcaatcaagtacttattgaccaccaaaaatgtaacttttagccctcaggctgaaatgaatttatactattttggaaacatatctgtgacatccccttcatgatccctcacaatgagccaagtctcacatggaaatagctttgtgagtcttgcaaccacacatatggggtttggtcacatttttacctctttcaaactgtttttttttttctacagaaaacaaagaggcaaaaaacttggatatttttctctatatgcctaaaagggcccatttccctctgaagttagacaaaatgaaaacaagccaccaataacaagccacttacaatgatcaatggggcaatatcatatgtgagaacacactgtgcctcttgcatattttcaaatgtctgtttcttccataagagaaaatttggaaacacatgtccctcctcaaatacatataaacagcatagactaaaacaaaataactacaatgacaaaacttaattcatgtctgtagattgtgaagcatacacttcacccagaggaaataaactaggctatttgtttttaagcactttctctattttaacacagtcgttttccaatgagagttttgttagatctcttatctaatcctccatatacaagcaacatagggttgctctctctaggcatgggaaaatcagtttaagaaagggaaggaaataattctcaagaagacatgttttatacattattcatccttgaaactgcttatgtgccaccaccaacaggaacacacatggtgaaatttgtattgtatttcaccttgaaaatccatattctgtcatagaagtccaatttcttgagtatcttttctcatccaaaagattattaaagtagattacagttgaaactgtaaattcagttatcatgaaagcagttttccccattgtaatacatttcattatgaaggggataaacaagaactaggaaagattttattgctattttcaagacccatcttttctaggatagaattcttcacttctaaggatacaggcaaaatagtatccaaaggctgagaatcatgtcatcaacattactaagtcacatgaagaatcatatatttaaaattacttgaaataaattattttaaatactgaatttttcttgaaaaactgacactccagaatacattagattaaggtcctccctagccaagaaaagtagagacaaggggaaaaaagaagaagaagaagaagaagagacaaagaaaagcagccaaaaaaaaatttggggaaagagaagttattgaacattgtcaacctacaaatatttattgaaattgcaactaatatgagaacgacaaccacagttataaataaagaaaagcgtatctatcagcatgtcatctgtcagtaattcgggcatatgttttcttcaaaaaaaacccacaatacataatttttaaagacagtttattcttaagcaaactaaagacagacagaggggtgggttttgtgtgtttgttgttgttgtttgtttgttttcattttggaccaattcagggtcactgaatcagcaatgattttcctcaccaatgcccatggggatatattaaaaagttattttttgaagaggaagtttgccttatcccaaatgttgtcacactatttctataaggtttttctttggttattattttgttgagggtaaggaaaacacacacacacacacacacacacacacacacacacacacaaaatgccctgtcctcactgtcagtggagaatccctgtggatttgtggtccttttcccattaaagtagtttcttccctgggtagtttttggaaagttatattatctcctgaatggccaagggtgagaatcttattccttccatgttctttttttaaaaaaagttaaactcaaaataactcatggcataaagaggggctgaatacatcctcagtttattgagtgatgtgaaaattgcccattgttatttgtgtcatttgagcatttcatttccataatatctgaaatgttattttttttttttgttctatctgctcattcgatgagcttcatgtagaaagtcaggaggcagaattaaaagcattgttcttgctgcatctaaaggaagctagcaggatacttgtcattttttagaaataaatttctctctaattttttttatctggttctgaaaggcacaatgcagagagattagtttttactggaaacctgagcactttgcacctcagaaactgaaacaatcgagtcctattttctatagcctcttatataagtaaagtcaaatgagttccaagttctggaaagcaacatgaatgtcttctaacctttattttttcagaacgattgagccaccaggctgtgtaggagcttcttcttaggtagtcacaggaagattagcttagaatggatgctcagagccaagacactagagaagacaaaacccagggtggaaactggactcctgcccagcccaagggaggctggtgcaagggacagtatgtaaatgtgacagaggtaaccgagatagcagagtttttgagatgagaggtcaatggtaaataaaaccagattgaaatgatctatttcagtagcttatggaaaaaactagattgggaacgaaagtgtatgatgcatttgaaggacaatgcagagacagtaatgccaagatgtggtaatggaatgaagtatcctaccctggtcatctatgctacttcagtttacttatatgtcaaaaccaataaatggtcttttaaccaaggtcacatggtgcagaaaagtatagaggatttctttattcctttttaaagttattatttataattggatgctttctcatacggatttgaaggagaatctcttcctaaagtggatataaacatatacccaatctccagtttctttcatttggccgatcttggtggtacctctacacatgaaatcagtgacttacaaatggtatcttgtgacatgttttacacagaaagtcttcatctcaaaatttgcagttagcttcccctgctttgagaaggctgtcatgaacttagtgcagaatttgttatatatttgtttttattgtcaaagagcttccaattgagatggtagaggtttgcacactccgttagggtatgttggatcattgctggaacgttgtgaagtgacactcgatttgatatttgttgtgcttagctggattactccagattacatcattgttcttgctgaacttcaggatctaagtcctcgctcttctacttaacagatgaatctttaatattgagtaagttacttaacctaactgtgccttgctttcctaatgtataaaatggagatcataatagtacctttctcataaagctgtctgtgaaaaataagctaatatgcctaaagtccacaaaacgtcatctgacacacaactctcaaaaatcagctgtcattatttgtattatcgttgttagctttatcattagggctttatcacctgtcaattactaactgtggcaccttgttcatttctgtgtgatacagcacaggaggtgtgaaactcagggcacttgtaaaacactgcttaaatctaaaagggactaagcataaaagtagaatacacatatgtagaggcttgtaaataacaaataacctgaagggctgtggtctgaaaatatacagctaaataagtggtggcttgagcatccctaatgaagagtgatataagactctcactataccatatagagaaaaaaccctaagagtactaagtggttaacttttattcccctatcacatttctttgttgttgttgttgttgttttgttttttaatcagaattgtgtagagcctacacttctgtttgccctggtattgtcttcttaatacttccaaagtgcttcctttgccaatttcaggatcatctcctgctgtatttatctcagatgagacttttttctccagtgtttgtttttaattcttttcctaaacacaccaaatcacaaccacacagttccataaactcaaactcattttctctgaggctttatcaggaatcatctcaagattgcataacagactcacttttaattgaaaatgttgcatccttaaattcctctggagaaagattgcagtattttcctacaaagctgcttactgctagactttctgatgaagtttctacttcattcagctggctttaaagactgtacaggttgacagttttccaggtgagccgagggtctttttgtcccttagtttatcaactgaactggtattttgacaaaaaggcctgtaattcctccttatgaggttttaaaatatatgtgaatcaaagtcaggatgtttctcattgggttgtgagtttactgcaacaggagtttacaacaacaggagttgcagtaaattcactgcaacaagtactgaggagactctctctgaaccctaaattcttccaagatgtggtctgcatcagcatcacctgggcttcggttagagatgcagaatctcatgtaggcttttacatgggcatcttgcgtggagttgaggtttggggtacaggtcctgtcactcaggtagtgagcatagtacccaataactggtttttcaacacataccttcctctctccctcttgcctctagtagtccccagtccctgttgtttccatgtttatgtccttgtgtgatcaatgtttaactctcacttgtaacagagaacatgtgatacatgttttcctgttcttgcattcataaatttaagataatggcctccagatgcatttatgttgctgcaaaggatatgatctcattctttcttatgtctgcatggtattccatggtgtatatgtatcttctgtatctgtaataaaaggagaaatttaaaaaactaaaaaataaaattaaaagattattatgcttaaataaataaataagaaatgcagaatctcaagccccacaccacaacttctgaatcagagtctcaaattatttctcatggacaaaggctgactggtgaaaaacgaaacttgtgtgatgaccccatgaagtgggaccctttccatcatttttttgtttgtgtgtttgtttgttttctctgtaattgctctttttctgaggaaatacagtttgggatggaggggtggaaagaagggactctgagtatatttgattcagacaaaacttattttcttttaattgaaatgataaggggagcagaaaaggacatgcttttaaatgaaccccatgtaaagatggccattgcagaaaaagtattcaattgcatacataaataggacttcagtggcgagcatccttcttagtgtcacatgtaattgaatttggctgataaataagccttgcatctcttctttatgagaaaaagtgcttatggattttacatgtaataggttcaagggttttgaaaagttctggatgatttgcatgcagtgtaaataatttaaaaatgtgaactattgttacgaggtgaaagcaagtatactatcaaaatttaactacgatgtttacaggcaactatatttttaaatattttatttgaggattatctcaagaccagaaaggcattaacataaactacaaatcatgaaggcaggaagtgtctcaaaaaaaaaaaagtattacaactaaatagataaattgggggaaaaagattactcctgaaatgtagatggactgttattgagctacatatcctgtaatatctgaatcatataagaccactttttgaagaaaattgttttatttaaattttcccatatgattatcctactaccataattttcatgtgatgttacagggtatatattacagtggttcattatctgaaattgattaaaaataaaatggtgatggctgagtagctcctgtctggcaaaaacctctcacagataacaactacaaaccctgaacaaaatataaaaagaaattatctgaagaccctggagagagaccaagacagggagatgctagaaagaagatgagactttaaaatggagatagttgtgagtagagttcccttggttttatccctttccaggacaagtaaaactctgatagaaaactctcaattttactgacttgaataatataagacagaatttagggcagctacagctccccaaaagtgaaggaaataaatctcaaaaagaaaagagtcagagaagaggggcctcagattctgtgtatatacttgtgtgtagtctttcactgaccacataaccatgtgaatgtggagtccatgagcacatcatacttgtttaatatgtattatttagtgtatgaatgaaaagatgatctaacagccatacccttcatagagcattccaacaagctagaatacctcatatcaaatgtatttactttaagaggtctgacagagaaaacagaacatctcaaggtaggattagggcacctttactaatctttaaaggaggtttttccattgtcccttgtttgacatttggaagttgtttaacacttgagtaatgaaccacagtaaactttgttccagatgaaaagtgttcttttatgttgtaaagtgaaaggatagatggaaaaggatagcttaaagaatgctacaaaatggtgcaaatggccatggaatattcacatgccccttgggcagcctttgcctatacccaaaggcacatacgcatgcccagtctgaagaccatagcgcaaagggaaacacctgtttgaccaagactgcaggggataaatgacagggtatagcagaccttagggactgccatcctctaaagaactcacactggaaacctagaaaaacttgacaaaaatttaagatgcccccacaggaattcatccagcattgtaagttttctattgatgtcagcagcctgaccgctgcagatatgtactgatctttgtagacatttgtaagaattgtagtgctgttaggtaaaaagttccctggaatttatttgtcagatcatagataagcagctcttgtttggatacttgataaaataatattgaattaggagttaaaatgttttcaatcgaacaacgttataaacactacccaaagccttattcattttgatttaataagcaactactgctcactttctatataaatattattgggtttaagtcctgagggtggagatggaagaggcagcagagacataaaggtgatgaatcaggtagaaacatatttaacagcaattacacattcactcaatatgtagtctagatatacattaaataaaacttaataaaaaaattaacctggccaatttaaccaacatagaccacaaatctttatcattcagtctctcttaaaatgtcttttccttcaggtaaaatatttagaaattgacttattgattcttcttcacctgttcaagtaaacatcacttcacactcttccctctgtgctaaagcaaactgataagaaaacatttttagaagcttcagggaaatgtaaataactatctttaatcattagagatcatgctgaacatatagaaaagtttatttctgttttgaaacaaaacttggcagaaataaaattggcagctttgattcagtgcaaagtattgtggagttggtactatttagaccatttcgaaataatttttaaatattttatttattaatatgtttgtatagtgggtaatgtggttttagttgtttgctgtaagtttccaaacatatttatttggccctctagtaactaaagaaacagagaagatactcttcaggctcttcattttgcctcagttttgtccatgcgaatttgaaaagtaaatcaacagtcagccacctcttttactgtaaagtagcctgtattgcagcccgccaaaaatagaaaagttggaataaagtagaatgtgtttgactttggcagaatctgctaatctgttccttataaattttcacacaaactgggtagattatttaatattaataacataagccccactagtctgatccattttttctcctctgacttaactgagaagagaatttttaactgtcttaaaataggctacaaaaattactttttttagaaatcattgtacatagtggcacttatttgtttatgtatctgtctccccaccggaaggtaacttctttgattgcaagcattgaactttatttttgcagccctagcccaggattggacacaaaaatgcaagcacagttactgcatcaaacttagccactgttttgaaaaaccaaaagagcttatctaaagtatgcattttaatcaaatcctggcagaatataggaaacatatggctgttatttttaattctcctttgccaaatttgttttacagcaaaacaaaactaaaacatctcacaaaattttccttaatgtgttattgtacctaaccatatgaaatcaacacagaagctgaatctaataccccagttttaccaatgagagggagaaattgcagtagttttttgtgtgtgtgtgtgaaagctaagagtttttgtgcacacgaaatgtatcatgagaattaagtataccctgaaaatgaaatatgcacaggcttggccttgccaaagttttcagagcgcttacttttggcaacacttccctgggtcttctctttgtgaacctgtgaagatggtgagtttcttaggagtagggactctgtttgacaactttggatcttctagcctactgcctgcaatacagtacaagctcaataaatatttgataaaccacattccaaatctgcattctctgtgatcccccagcccctcaattttcctactctatggtttcaaagagtgataggacaaaatgtgtgtgtagtcccttcctgaggtattggcattttagtaaggaaaacagctttaagctaaagaaatgaaaccgtatggcatgtcaggcattctgaagatgatggaaggcattctgaatggcaagatagtattccttagctccctgttatacatggtaataggcctgtagtgcaaatgatgccaagaatagttcatggtggattttcaattgcttaaaagtaggacataggccatgatcattgcttttcgttgtttccacagatcagctctggtctcctttctatttctcatacttaaggtcttcctctatttctcatacttaaggtcttcctctacccccaccacaacagaaaaacacctcaaaacattgattacctattataaggttggtcttgtttctcaaaaacaaaaattccaaagcagtttacatttggaggcactctttgctatcgatggttcttgaacatattgccacacaagatttcctatgttatgtagattagaggaattttcttaagtgtccttgaaaaatataataggaaataaattcactactgaacatctatcttctatgaagttttgaggttgacttgatgtattagactaaggaaaacctgtgttctataattctgattccatttttaatcaaattattataaatgccctttggggcattttgtggaaaagatagattaaatagtgtacccaattaaaaaactgaaggatcccttccgcttgaaagttacatactgctcaacaaactgtatgttttattaaaacatgaactggtagtaagggcactgaatagtgatatgactcattgaagccaaagtggataatatggctttcacttacatatcattcacagtggcaattatctctttttatattaatgaagaaaataaaatggagtataactcacaaattttatctatttttggcttcaagtatttcttgttttacttggaagtgatgcatttatattatttgagtgcagatttttgtcttgcaatttggaagatcatactacttaaacaaaattacactggtgtgacttatctttagaaagctagtggaaagttgcttagcttatttgatctgatctgagtgacttccttaataccccatgctgctaattaagaaaccatctcagaatgaaggtaaggactaaccctaatcaagaggccccatcattttgtttaatgcctggtttactccacccttgacagaaaggttataattaagtcaggggctatatgacaatatcttattcatgttgatactcccagagctagcttagttcctggctcatagcaggtgttccataaatgtttgttgaatgcaactgaattaccttgaatatcttgggcacaggattcttaatcaggggcctaaattagaaattctgaaaaatagtttgaaaacaaaaagagagatgaggtgtggttctgtatatttcttaaaaaattatcaggtgacactgataaaccaccttagttatgaaacaccgatctaagtgctaaactatttttgtgaaaaattctaatagtcagcttgatcacctagatatttctaattattttcatgaggtgtttcttttaaaaatgaatagactggtgatttttaaaatttgtgtgagcttttgaggcctttagcctccaagcaatatcttgcgtcaaacccaaatatggtcaattctcatttttattcatgaattccacctttgcaaatttgtcttttcctataaattgtttgtaacccccaaatcaatactcctagcagatttgctatcattcacagacattcacagaatgtccagaaattggagccacctgatgtacagctcccagctgaggttaaagaaggcaatgctctctgtgccttcttgtttcagttctcatactacgaacaaatgtcctttttgtggtctatttagtgccacaatttttacatttttatgaatttgtaaattggttggtgattttattgttttaaatagcccctcaagcatagtactgaagtgctgtatgatgttcctaagtacaagaaagctgtgatgtggcttacagagaatatgtgtgtgttggagttttcttcaggtagaagtaatagtgctattggttgagagttcagtgtgaatgaagcagcaacatatgttaaataagttgtctttaaacaaaacacataaaacaaagttagctattgattgctgggtgaaaatatttgtgaccagaggcttacaggaacctaaccatgtgttttccctagaagcaatggctcagtatttgctatttcagtttttgaagcaactttatagaccataactactgaaaataacaataattaactgtatataaaactggaagccaaaattgctctgggtgacaaaggcataagaccatcatctttgttctcttcccatctccttctccccaagtgacagcctccaaaacatatgcttaatttctggatccagaaagaatagggcttttaaataagaatagccttccaaatgaatccagagtaacaactgaatttcacattttatttatatacttgggtgttatgtaaatatagcaagtacttgcttagaaaatacaaaactaacattatattaaataatcccagttaggaaggtaatcagatggcggggagggaagccagtgcttttctgagccaaatataaattacttttagattacctggtgtttttaattatctgcaccactgtaccactttggtcagaatcagaggttgattctacctctgacaatgccatatctgccttctcctgggcccagagggcttttccctatgttcaataaaccttgaaagaaaaggaaacacagtagaatatctgttggctcagcacaacaggttttgagtaaacgaattactactcttatgcattaaataaaatatatggttcggaaaaggttgctacagtactgaaagtcacttcatggctatcttgctttttaaggaaaaggcctaaaatgcagtcaaattcaaaataaagtaagaagcagcataatgtaaaacagagtatttggtaaaataatagtaatgcaaataaccactccctaatacctaagattttgaatgctaaattccgttgtcattattgttgttgttaagacataagcacagctaagtaccctcagtcacgcatcctcaactggggacattttgctccccagggaagactgggcaacatctggagacatttttgattgtcaagagtgtgtgggagatgccactaacatctagtggatagaaattagttatactgctaaactcccaacaacatacaaggcagcccccgcaacaacaacaaaaatctactccaaaatgctaatgtaccaagatgaaaaatcctattccaggagtgactagtaaataaaagtattccattatgcagtacaagcaatggttatttctatacagacaacaatattaatctgactcttccaagggtgtggctcctacgtctgcattgagttctcagcaaggatgtcatcctaatctctgtgcccagagagcgcttgatcccaatatacaaaccaaggctcccatatgaaaacaaaagcataaacagcagctaggctcagcgtatctcctgggtttatatatgaaatttatcacctgactctctcctctttactgctaaaggaacatcatcaacaaatcagtgaatttcagaagcagcattaaaaatatctgcgttatttgcttcactggccatagaaaaggggccataaaagtcatgcatcaaatccttagatggtgcctaagattatgagtctgacaaattgcaggaaaatgaacccatctgtgtaagtccgcagaatgcaacaaggaagaacattttgtggactagttttccactggcttatttggctatttacatgtaatagcagtgatctgtgcattcactctgatttgtgggtatagcaggtggcacatgtttacagatgcttgggaaactgccttgcaagctgaatgcagatactttggcttgttttcacatttgctctgtgctccctggcagatctttacctgtgtctttctctgagtatcttcctttctttctgacaggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctggggtatgtattttctgcagctaagttttgagtcactttcacctctgtctgtatgctttcagtcttcttctagtcttggtctcagagaatagagtcaagtcccaggtccttcaaggaatgatggaacacaggtactggcaggagttcagtctccaaaagacctgtggaagccctttaacaatctcaactgtgctgaacatgagggtctgcacagagaaggaagataagggtgacttccaaagggtaacttcaggaaacaaacctagctaatgatggacacaaaatgtgtcctgattgagagaagatctgattaggcctccttctgctttattcatcacatgtttgtagagcaaaggagaatttcacattttgaattatgtacctcccctaccctggcttcattttccttcatcaagaacatttcttcctgtcaatccatcatcaggattcctccagtgaaagctcaggaagtggagtccagctgagaggcagatataaccacacgggcttccaagtagagcctgtgtgacatcatagcacatggcattggctcgggaaagcttagggctggattgtttaagctgtggctgcttccgtacaaactaaggcagggtgctgaagtccttcagggatgctctagaattgctccctttcccccagggctgctgaaagtccagctccccctggtggaagggaactgaacagaaggtgaggcaatgaaagaaaataaatgctgcgataaaaacacatgctgccttatcaaaaaaaaagtttcagccttaccttgaaacttccataccagaggaggaactcgtgggccatccctcactgactatagagccaagggcttctctcaccacatccaccccacatgatgggtgactgtgactctgagagcagaggctgtggatggccttcaactaagcttgccctttaatgcagcaggctggggagcccctacaagtactggtaagagaggagaggtgtagtgaggagggaggacaatccaagggattttttaaaaacatcaccttttgtatttaaagccctgcccaacaggcattgtcgccaactttatagaaatgtgcttgggaagtcatataagcagcctagagtctttcctgcagtctgtaaactgcagaaaggggctggtgttatgccatactggtttccataacacccttacataacaggggtacatggttttaaccaaatgcattgcccaagcccaatgggataatccgataatggtgggtcaatggggcagataggcgggatgcctcaaaattgaattgatgatgctacaattatttggttgcaagcaacagagtaagatgttctaacttaagcaaatacagagaagttgtcagaatctagcgtatctcatggaatctaagggcagaaatatagccaggcaactaaaagggctgtagcctgggagcagataagccaggagctctctccctctgccttttgtctttgtttttccttgcatatctgtttttacccttctctttctttacaggccatagaccatagggtctctgctccctccagttttcatagtaaatacatcctgccccaaagtttcctgagtacacatgtttctgttccaggcactcacagaaactaaatgcccatctttaagttccaattctgaatagcaggacaagaaaatctgattgatccaactgaaggcaggtagcctaccctggttcaataaactgtggccaagaaaattgagggggatgttgtcacatagtgtgaacatgtctagtgattgtgaaccctccagcaaaggactgagaaaggggtctttgtgaactgtacaggctgtctaggatattcaccagatcacctcatctgcatggctgctcccttataaggagagcagcgctgcaccctgtttccttaaccatccagtgaggttagaaatcaggctatcactatcctgctggttccacatcaggtaacaggttctcctgctctttcctgtcctaaaacaggtcaaaatttactcaaaatggtcttttactatttttttttttctcttttagccccagccttccacagagactaatctttcctttccctggccttctcatccgtatgtacaccctctcaacaatttctagatttccaaatctggtcatgctaagaatagactggatacaatttaatcactgcaatggcaattctgatcatcacaatcataataattggtgttgatcaaatgccttccagtcaaagacctgaaaataacattccaccccaaacacacacacaaacacacacatactttctgccagatttatttcctttgaaataaaatctgtggtatatttcttttaccagttttgattgttcaaaacatctatctgccatactcagaaaatttttcatgggaaaaaaaggctgtaaatgtattcttgtcggtgacattcagcagagacaagggcaccctggaaggaacaatttatactgctcttggagtatactgatgcattgtgaattagttataaaactttttggcatcatcatagacagctcaatgaagatatctgcttaaaacacataaacaattcaaaggaaaaaaggaacaagctgctcagctgtatcaaaaacaaaggcaataataataagtaaattgtttatgtgataccttaagcttctgtaatcaacaacaattcgaatgattcaagtagaatcccaaagaacattacacttagtaacgtaagcacagaacggggagttgctaatcaatggttatacagattaagtaatgcaagatgagtaaattttaggtatctgctgtacaacattgtgcctataggtaacaattttgtactatgcacttaaaaatctgtttagagggcagacctgatgctaattgttcttgccagaataatataaaaatttaaattaaaaaaagaactagagggatttgcacttccgtagatggacttttggaaagatagtcaaccaaatagttctaaaatgtaatgtttttagcatgccaaagctaagactatctttctacatgcacacacacacacacacagacacacacacaccaaaattccaccaaactcattaccactgaaaaatcacattgccaaatctgaactctgagaacaaaattgacatattttaaagcagtggctcctttcttagaattttaagcaagcattatctttggatgggaggaaaatgggtgaaattaataccaattaatttctcaagaaaaaaatagtttcacagaaactttataaagtttaatggggcaagagaataagtctcttctggataatatttttattgtcctttgaaaatttttttcacatgaagacagtatgataggttctaaattttacaatgccagaaaagaagcaaaacctacatacttttaaaagattcttgaaagttagactgcagaaaatatggcccaaaaaagcccagttgtattatagcacctaatttgtttgcaggtgatgtaaagcccattcatttaattcagctgtgaataatgtttccaattctaggatttgaaatgaatctcaaagaatgataagagatattgtaaacattagccactgggtgaacaagtatctggtggtgaaattctccccaacagaaaatcaaagaatggttctttcagtagttattcccttccaagtctgcatggcaccacttagtagtcgatttctctccaattgaagtaacttcaaatactgaagtttagttctcaggaagatcacttattttgcagccctctccagactgtttctttgccttgccttgctggttttccccttgttagaaatcaaaggaaggaaatttcccccatggacttctatattgaacaaatagaatttattaaatccctcttaaaaaatgaatcctacacagacaggattattctgaatctcatcatctttggtgggaagaatgggttgttatggatcttgggaccttggggagagagaaagaaaaaagaaaaaaaaacagaaaaagcaaactacctaaaaggaggcatcatagctagaagaaggcaaagagccctgggcgttaaaaggtgtggggtctatactcgtctccagatgaagtagaaagcactttcctttctacaaaatgatgaggtgggattcagttttccatatacttctctttctctctaattctaaaatatccatctcaaaataaatccagagactttgtttctctttctgagcccaagtgggaaaatggtatgttagaactgctgtgaataacacaatcatgttttgtataagcaaactcaaaattatttcaggccaaacattaaatctgtggtcctattgaaaatatgcttcctgggtttaaaagctgaggttgttcttaggtgaaaattcaatattttgagacccctttttgtatataactcacagaaaataaaaatgcattgatttaattctttcctaagacacatttcttaatctgatgacaataacaaaacctctttctacagatgtctcaatcaaatcaaacaatattctactgttgtacatctgagtaaaaagacttattttttcttttttaatgaaaattacaactttaggttatactacagaataaaaattgatttaaaaaattataatctggaaggacgtattgcaataactgtgtccatttttctgtttataaattaaaaagtttttttttttttactagcactttgagtttagtcaaggaggtacagtaagctgccttcataaagtttctctagccagaacctacagaaggtagggaatgtactggcttctctgaagtattcatattaaaaggaaacaatcactgcatgagatataaacagacagaaaaacaaaagagcctagaagaaagaatgtgaacatcagctaaggctttaggagcaatcagactattgctttcatacaccgtcccaggtcacatccctccggggtgacccagctcctctccataagagaggccagttcacccacaatgaaaggctttctggtctcagttaatagtcatcattataaacataacaatggtaataatagtcatagtgtacctgctacattcagcaatttgtaaaaggcttttaattatttggatgctcttaaccaaactctcaggaagcacatgatgttctaactgggagcattttgactggcttgaccaaagatgatttttgtgaaaatatcaggggaacatcttgacagatgcactcaatttgccagtatttttgcaagtacactaccattgtccatgcagctgtacttaaaggttatgaccaagaaaccaagtgtgatttgtacttttcacatttgtagggttcccgctggtgccaaccactatccatgctattaccagggccgtgtacttcaatgacaagtaagtattatggtgatgtttaagttaacatattcataaacaaaatgtatttgtgtattaattgtccccttgccctttttcagctgctggctgagtgtggaaacccatttgctttacataatccatggacctgtcatggcggcacttgtggtgagaattagttttttattgcaatattagtagtattatctcttagttgagatttctgtgtatatagttctatgtatcatatgttttataacttcaaaatgaagctaatttgatggtattctgtagtaaaaaaaagcaatttgattttagactcttcaaaaaaacgccttttactcacacatggaggggttatcatgagctcattttcattacagactcacactataatagcaatgtagctcatattttaagatttttataactagattctcctaggtaaaggtgagaggccaaggggctgcaagaagtttgtttgtgttatgaacatctagggtctttgaaaactttgacattccagagctctaaaagtctttggttctgtgaacttaaggtccagaaatccaaatattcaacacaagctcaattagtgagttagtaataaatagtggactcccagccaaatgtcagatgcttactgaggatgctacagttcttacttacctaggattcagtagacttggcctcatgtcttagctctatcatctaccagctctgaaaatttagataaatcaattaacctaaaaatgtcccctgatttttttaagggaaaagttagattaaataatctttcaggtacttttggaagataatctctaactctaggtcattaaaacttagtactgctccatatctttatctgttaaaaataacaaaaataataatagctactcataaattcacgattataaaatatctcctaataaaataataaataaagtaaaatgaagtaaaaactttagcaattattcctatatttaaaattctaaaatacatatttcttgccttaattatctagaaatttttaaggaaatgcagattctaggatccaaattataaatgtgtatctgagttgagtatatgcatacttcactgtgaatgaccataaattaacagaaattgatatatttggaattcttattaatttcatgactaaaggaaatgcacattagtgcatttcaaaactaaaaaaaaagattcaacaattaacaaggtgaatcatttataccttaatagttgatttttctcattttgaaagagctgcccgtgcaaatatgggcacttgatttatgacagcagcacttttgggcagaggaaggagatggtcaataagatatacatacagaaaaaatatcaaacatgtatttagcaactgcatatgtgtggggaaaaagtaaattctgattccttccacaaccagacaccaaaaaatgtgtttttagtgaactggaggtttaaatgtaaaagacaaaacaatagaggccccccaaaaagacacattatgttgagtatcttcatgagcttcagatacagaaagatttctaaagcaggatacaaaaggcacttaaggaagagattgataaatcagctacattaaaatgaagacaacatttaaagactgaaaagacaaaacttagagtccaagaaggtatctgcaatgttccaaataaagactaataaatttatgaaaaagtgttcagctcatgtatcatcaggaaaatgcagattaaaacaagaatgaggtaatactacacccctccaccacccccgccatagaaaggctcatattaaaaagagtgttcataccaagtgttggcaagggtgatgagcaactggaactgttttatatactgctggagggaacataaaattaaaccacttctctagagccctagaaatgctgagccaattgtgctacctctcaggtagactctcatgttctatgtaggtaacatttgttgctcacactggataaacaagaattgagaacagtgcatattcttaagtgatacttttctgtatgatttgctcataacaccgtatatttgcaggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccaggtaaggaagccaaaggtgtatttattcaactactgtgcttattcagctagctgattttaactctaaagcccatgatcttttattattattttacttatttgtttatttatttatttatttatttattattttattttattttttttgacacagagtctcgctctgtccaccaggctggagtgcagtggcatgatctcaactcactgcaacctccatctcccgggctcaagcaattctcccacctcagcctcctgagtagctgggattacaggtgtgcgctaccacgcctggctaatttttgtatttttagtagacggggtttcaccatgttggccaggctggtatcaaactcctgacctcaggtattctgcccgcctcggcctgggattacaggtgtgagccactgcgcacgcctgacaagcccatgatcttcaatgtcataacatttaagcctccaattcaataagcccagccaaaattctgaaggacaccagtttttttgaggctctttgttgctgtttctttctgtgcctgctttcctgtcattcccagggccctcagctggtcccatccaggcatgttctccttggtcatagatacagaaattgtcagatcttcctttataaataagtgctggctctggggtaatgaggaatttgcacaaggctgtccatgcctcctgctctatcactctccatttgtcaggtgaatgttaggaatccattcatgacggttcagtgctataaagggaagttgtatccaggcctcccctcatgtaagagagaatgcagtgggctctcaggggctgtcctgggctgtccagtgtctgccgcactggataggagaatggctgccctagttccattcacctttacccagatctaaagccccattgaaagcataatgctgtatttggatagcaagataggatgtgggagtccttggcaggagtaattctatcacaggctcttaggccagagttttgctgtctctattacaaatttcaaaatagttttattttctatattaggcttaatgtgaggaggaggtcaaaattaatagggccagaaagaaagtttagggatcgtttgaatagttcgaactgttcttttagccataatcaaaacttgggcacaagaaatcttcaagcagtgaccaggactcaggcagtttcttagtccatgtccttatctgagcgatgaacttggcggtgggtacagtattgcactagttcaccatgacaacataggacacagacccacagcctcccactatgacacttgtcttgtaaaagatccctctactgaattctgcaaggtctccatttttaaattttctaagtgtagtttttttttttcaaattcaccataatcagcatatgctgctataaatctccatgttaaagggagcaaaatattatttagattagcaaaatattaatccgtccttctagccaaacgatggaatcctaacaagaaaaatatatttctaagggaatattcttatagtgcagcactgactatgaatggtagataattctttttcttgctttttttttttttcatcaggccttctctgggcagtctattatattgtttactaaacaacataatgcacacaaaaataacttcaactgtatttttccttccctttttagggcttctttgttgcgaccatctactgcttctgcaacaatgaggtaagcatgcatttcatataatactatcacttgtttgtgtaaaggcagcattttttccaaagggcctctgtacaaatttcatttcatctcacttctacagttgacctcatagggttttgtgattagcaacatgatgttttttccactactccaagctgaatcttatccatggatgtgtagtacaatggcaaatatgattcagacaggcagttaatttccttgggttgcatcttgaacagatcagcaccctcaggagcaatagtgaaagctattctggatgccatcacaaagagaagtcatattcctattcccttcggaagacccgagccctcactagggcagagacaatctccattcggtttgtactagttcctcaccttcttttccagttgcttggtgctaacgccctactacttactcagcgttttgaaaatcaccctgaagtgaagattcaaaaaggattctcaaataatctacaatgtgaatatttagatcagtaaaatctagagagccatatggctgatccattctgttctcagggtgtttttagccagagttaccctttcttccaaccaaatcttgcctgggagccaaatctataaaacataaaaatggagcttctatagctccagacatccttccaaagaacttccagggttttagggaacatgattcaaaatcttcctcatttttcagggaagaaatctgaactccagaaagatcatgtgatttacccaatgtcacaaatgcacttatggttataacccagactggaacacagatttcctgtttttcaattaaactgaccaactcccaattaacttatctatatctataattccttcctggagctatgttagtatgttttttctaggaccacacaacttttaatgccaaagcatttttgaacattttaaatcaattcctattaagaataattccatacaaggcaaatcacattttgccaagacatcaatatattactttcctattagtaattagaaacatttctagccattgtcagttgctaatttattctctatctttttactaagttaaataatatttagtgagcacctacaatattttgctattctgtgacctgttcatacccgtttccaaccgcaacatgacaaagtttttgttgttcctattctccattttagttattacattttattcctttctttcttcatccattcacctaccaactcattccttcattcaacaaaaatacattgtgcatttactattgctaagggctagactaagggcctagaatagaaatgaaaagacatccttccattgcccgctgttagagtggcaatatttttagaattttaatattttttagaatgataaaaactaataaatgagtagggcgtaagtcaccccgtgtttatatcaatttctgtgaatgtatatccatgccttttgggttgtttcaggtggcatgcaaaataaattttttgtctaaaataatatcaaaatagagctaatatgagaaatcaggctcagtggggcatacctggaaaaacacaagaggattgcatgcattcgtaaaaataccacaatatttattgtgtcttatctatgatactgtcattggaagtgtcagacatagaaagcaaacccaggggtaccctagcaattgaatgtctcgagttcatttgactatatgttatctctgattttgaagtaaagaaaccatttccattttcattaagatcccgttactgggggaaggtaaaagtgtgacatttctaataatatagaattgtgtactgtgccagatgtaatatcacagacccatggattttaggcatctgggtctcagctggtcattctgcgaatgcatcaccttacaatattgaacacaggcagggcactcacacatgcaggaagttatttccagctgcatctaagatagggctgtgatgggatggtgcctggctgaatactgagttttagcagaaatctagggggctaactcaacataataatgagatcacatcacattgtgttatgtgcaatttaattttgattatgattggtatgttatcagtgaaattattatttcatttttgaattttgattcattattggtatgttatcagtgaaatgatcattatgaaaatgaattatatagagagtcagtgggtagactagaagcaggaaaggaacttgaagaattaaagtaatagaaatcaggagaggaccatgagagcatgcttatgtatcaagcatttcctacaatgactcttccaagtgagtggacattgattatccaaacatatcagtaaattattaggtacctaagagacctgaaaaagatgattgcagctataaaaatgcacaatttgtttccaagaagtagcatagaaatgaaagttctaacaacacactagattttctcccttttcttgtgtctttctattctccgtacatcgtggataaaatagtttatagaaggaagtagcatgatcagaaacatctccatttaactgtcacaaattggagtgtgcaaaagctaaaattttgtttatttcctgtagcttatcttgctttcaagagactattccaccagcctctagtggtttacagcagtctctttatagaaaatattcagtcaattcaaaaagcctttgtccaaaagagcaactggctaccaagttttaatgccagcacctgctgttgaagttcacgctggttccccttactactaagccctcgagaagtcttttcacctgtggcctccttgatgaagccacagccctattctgattcacaagttctttccgccaagccacccgaggagcctggaactccagctcctttcttcacaccacatagagcctgtgaatctatcttcagcacatgccatagccgttgctgctccgctgttggacatggtgccaggggcaaatgtggagtttcttttcagggctggctattttcaggactgtgctctcgaaacaaaaagaaaaacttgatgtaatggagggttcctaagaaagcaaatgatcttttgcttgatttcttctgcccattttcccaggtactcccaccacgaagggaaatgcaaggccatttgtgtgtctttctctcctcttcctctcaggcctcttggggctgtgcactcaccacttccccttcctgtctgtgagggtctctacctatatcatattctacatgttctctaccctgtagcttttagtaaaacactatctgcagaccagtaagattttcacaacatatgcaaagagaagtttcttctgtttagaaattctcccactcaataaagcctgattttcaatacaatgtctttctagagacataagaaagccagctttgagtctcaatgctaagcaataattttccttgttctagaccagtattctcaaacttgagtgtacatcagaatcacctggaagatttctgaaaccagggattgcttggccccagcagcagtttcctattcagtaggtctggggtggggtttgagaatttgcatttctaaccaagttcccaggtgatactgatgctgctgacccagagacttcactttaagaaccactattctagacacaatcacacttgttcttcaagcagtggatgaccttgacttaatgaactggggagatgtaggaaattttatttaagaaaattaagaaatacaatggctgaatgttttcaaaaagtttaactttatcataaatgatacttaggtattgttaccctttagagcaaggaatcttccccaaacataagaaaagagataatgaatgggaatcagaaagctctgattctataatcaagtactcaactcaaattatagagcatataaattttgtaagataaaatcagagaccagtagagatattgctgctatggcctccgaaagactgaaaagaatgttgacaacaactttgtaaataatattcacaagtatcccttagcatctaattaatagggcttaatacgaatctagctttcagaagttgtataaaaccacctaggccaggcgtggtggctcacgcctgtaatcccagcactttgggaggccaaggcggctggatcacaaggtcaggagattgagaccatcctggctaacacagtgaagccccgtatctactaaaaatacaaaaaaaaaattagccgggcttggtggcaggcccttgtagttccagctacttgggaggctgaggcagaagaatggggtgaacccgggaggtggagcttgcagtgagccgagattgcgccactgcactccagcctgggagacagcaagactctgtctcaaaaaaaaacaaaaaaacaaacaaacaaaaaaaaaaaccgtctaaagtgtgcaattactttttaacttctagggaatttaaagtgtttttttttttttaatcatgatttttaaatgtcaagctcaggttgcagcaggtctgttagattgtggtgacagtgtgaaggagtgggcgttacaatctttgcattcattggatcaaaactgcagaggtaaacctcaccttctattccgcagtacataactatagtaggtacttagaatgaactcaggaaaagaaaggcaaaagacaaaaacattcaaatccaaccatgaagactctaaacttattttgttttgtacaaatgtaggaactgaggcctaaatatacaaaggccaaatattacaaagcttattaatagagccaggtataatagttaagtcttcaaactgtgaggccagggctctatttttataagttggcaatacataaatagatataatcaggaaaaaaagtaaagtgaatagtgtgtattgcttttttagtgagtgatgaatttatataattcaaatctttttgttgttgttacatttatcccacttacaactgaaaacatttattttcaggagcaacaaaactctccctgttttgttttgttttgtttttcagattaaatgcccttattcttgtcatcagagcagcatcagactcaacctgtgatattgtaggagataattgcccttattagtctataattttttaagtgtcttttttatgctgctaagggcctgttattatatgaatagtttacaaatcaaagcacattgccatctccacacaaaaggatgggttgtaattttggacgcaattaaagaaggccgtgaacctttctgtaataataacttttaaaaattaaaatagaaatcaaaccgtttggccccatgctgggctgtgtactttattaatataaatggatactctatgggagttattttaaaaggacaggtgagaaataaaagacctctttttcatgctgcatacttttgcaaactaactctaaactcataactgcctgcttggatacttgtaaatatctgtgtaagtttcagctacaaggataatcttggtatttaagagatgctgaatccgtggaaattatcagcttcctttttttttttttttttttgagacggagtctcgctctgttccccaaactggagtgcagtggcatgatctcggctcactgcaagctccgcctcccaggttcatgccattctcctgcctcagcctcctgagtagctgggactacaggcgccggctaattcttagtatttttagtagagacagggtttcaccgtgttagccaggatggtctcgatctcctgatctcatgatctgcccgcctcggcctcccaaagtgctgggattacaggcatgagccaccacgaccggcctatcagcttcctttgactggagacattgcttctccaaaaatgtgctgaacctgtatatttcccagtttctcaatggggcatttgatggttcctagattataaccctggcatttaaagctacggtaatagtaccacccaaataaagccaggctctgcttggtactgtggctttgccacattctagctatatgattttggacacattgcataacctcagtgccccatccataaagtgaagatagtagtgcccttcccatagggctgttgtgaggattaaataagttaatacaaatcacatacttatgatacggtacttagtaaacactcaataaacagctgaaacttgttattctatcttttgttcttgcgtggcttttttcaatacaactttcagctgcatggtccaccttattccttagcacttaaagtttttgtctttgtccgttttaaatcactttagatataaagtacccttcatgcaagcactcattaaccccaaaacaaaataaaacaaaactcaggatgtgttgcctccataatctgaaaagtctaatattccaaattatcagattgctaaataattctgattgccatttgtcacctcgaaccttacggttgctgtgctgtgtgaagataaactaccccctttagttgctttttgaggaatcatttgactttgtctttctctgagtaaaatctttaaaaatatatgaacaatactaattattctcagaggagattattactctactagatacaggaaaaaaaaagtatatttgccacagaaccattcagccttatattatacatatagttttataacattaatcagttttgctgtcatttttctcatctctcagatttgtacaaatgtaaagtagataaaatgattccccatgatcaagtagttacatgatttagttcacattatatattccccaggttctctctcatgtgtttagctgtcatatgtgagtttggcagcagaatgaactaaggatgtatcataaattgatgcaaatatttttatgtaagcttgttgtatgtactgagcaatagaaactcagaaatacagaatagagattattgcaataatctcttttttagttttgttcagctatagagaaaagcaagaagtttggagacaacaggaaggaggaacttaactactaaattgaattcgtttctataaaaagcagagctagacaagcaaattcactgtctgtggtactgttttcagttgtggttattgtagttttcatatttagagctttctgatgttagcatctttgcctcttgttttcaatatataatttatcgtatttataaaacaagtctggcaagtttgtttacattttagaagtctttcaaaagaaagagaaaatcttattcttaaaaagttgtcatggcattaaccagagatgtatctggcataacccatctcaagtcatgtcggacaaggaaaagttcactgtacgaggccattatcataaaatccagaactgtgctatggtttacatatatatttttcatgtgcacacttaaattaaaaacttctgccttatcagggatggtagcatataaaaagtttggtcaacctttctccctattatgacatgtataccacattacctgtgaaaccaaatcaacttttattcattggatatgaaagctaaagaaaccttttcttaaaactagcaaacaaccttgccaggggtaatttaagcaagtaactcagttcttccatttgcaaattcttcttccaagttgatatattctgtggccacagcatacacagcccagccctttagatcagaattacaatttaactttctttaaatctgataagctctaagaacgatgttcgtacactttgcatggatcatttctgactatcatataaaaacaaggctcctttttctcttccaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatatttagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaa

SEQ ID NO: 2 - RNA, 3331 nt, Homo sapiens
  source 1..3331: mol_type = mRNA
cagaattccaggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaaggcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatatttagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatcttttcattc

SEQ ID NO: 3 - RNA, 3588 nt, Homo sapiens
  source 1..3588: mol_type = mRNA
gtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccagaataaatgattcccactgatccacccacttttgccaccccaggatgcaattttctggagagaagattagtggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcagaaaattgacaactatttttcctttgaattggaaatataggaaggcattgagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatattcagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatctttt

SEQ ID NO: 4 - RNA, 1674 nt, Homo sapiens
  source 1..1674: mol_type = mRNA
cagaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttg

SEQ ID NO: 5 - RNA, 1512 nt, Homo sapiens
  source 1..1512: mol_type = mRNA
ggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagccatcatttcctgggagaaagaccatg

SEQ ID NO: 6 - RNA, 1472 nt, Homo sapiens
  source 1..1472: mol_type = mRNA
atgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaaccgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaaggcaaacacagcatcgtgatcactgagccatcatttcctg

SEQ ID NO: 7 - RNA, 1506 nt, Homo sapiens
  source 1..1506: mol_type = mRNA
gaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagccaccatttcctg

SEQ ID NO: 8 - RNA, 1493 nt, Homo sapiens
  source 1..1493: mol_type = mRNA
ttccaggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagcca

SEQ ID NO: 9 - RNA, 1496 nt, Homo sapiens
  source 1..1496: mol_type = mRNA
gaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagcca

SEQ ID NO: 10 - RNA, 1339 nt, Homo sapiens
  source 1..1339: mol_type = mRNA
ccaaaaatgaggacgaaagaagatgatggatgcacagtacaaatgctatgaccgaatgcagcagttacccgcatatcaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgacccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 11 - RNA, 1772 nt, Homo sapiens
  source 1..1772: mol_type = mRNA
agttataagagacaggaataccagtgagaagtatgagagagtgggtgggagataatgtttagaatctcttttgctgcccgcaatttatgaaaatggcttgaaaatatttatggtcaaagaccgaaatatttcttcaaagaagattagctttgctccattaaaagtaatgagtagaaatattaaaaaaaaaaagttttaagtacctgagtatcttgccagcaactgaccaccactgctaaaggtgaggagagacacagctttcatcattgggactgcagtttatttcaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 12 - RNA, 1530 nt, Homo sapiens
  source 1..1530: mol_type = mRNA
ttgcttctattgagctgtgcccagccgcccagtgacagaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 13 - RNA, 1735 nt, Homo sapiens
  source 1..1735: mol_type = mRNA
acagctgggagagcgcaggaaggcgccgggaaggaaagccagcccaccagcgtctgggtgggctgcgccgcgcggctggcggaccttcccgggttggagaggtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 14 - DNA, 3331 bp, Homo sapiens
  source 1..3331: mol_type = genomic DNA
cagaattccaggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaaggcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatatttagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatcttttcattc

SEQ ID NO: 15 - DNA, 3588 bp, Homo sapiens
  source 1..3588: mol_type = genomic DNA
gtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccagaataaatgattcccactgatccacccacttttgccaccccaggatgcaattttctggagagaagattagtggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcagaaaattgacaactatttttcctttgaattggaaatataggaaggcattgagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatattcagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatctttt

SEQ ID NO: 16 - DNA, 1674 bp, Homo sapiens
  source 1..1674: mol_type = genomic DNA
cagaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttg

SEQ ID NO: 17 - DNA, 1512 bp, Homo sapiens
  source 1..1512: mol_type = genomic DNA
ggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagccatcatttcctgggagaaagaccatg

SEQ ID NO: 18 - DNA, 1472 bp, Homo sapiens
  source 1..1472: mol_type = genomic DNA
atgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaaccgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaaggcaaacacagcatcgtgatcactgagccatcatttcctg

SEQ ID NO: 19 - DNA, 1506 bp, Homo sapiens
  source 1..1506: mol_type = genomic DNA
gaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagccaccatttcctg

SEQ ID NO: 20 - DNA, 1493 bp, Homo sapiens
  source 1..1493: mol_type = genomic DNA
ttccaggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagcca

SEQ ID NO: 21 - DNA, 1496 bp, Homo sapiens
  source 1..1496: mol_type = genomic DNA
gaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagcca

SEQ ID NO: 22 - DNA, 1339 bp, Homo sapiens
  source 1..1339: mol_type = genomic DNA
ccaaaaatgaggacgaaagaagatgatggatgcacagtacaaatgctatgaccgaatgcagcagttacccgcatatcaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgacccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 23 - DNA, 1772 bp, Homo sapiens
  source 1..1772: mol_type = genomic DNA
agttataagagacaggaataccagtgagaagtatgagagagtgggtgggagataatgtttagaatctcttttgctgcccgcaatttatgaaaatggcttgaaaatatttatggtcaaagaccgaaatatttcttcaaagaagattagctttgctccattaaaagtaatgagtagaaatattaaaaaaaaaaagttttaagtacctgagtatcttgccagcaactgaccaccactgctaaaggtgaggagagacacagctttcatcattgggactgcagtttatttcaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 24 - DNA, 1530 bp, Homo sapiens
  source 1..1530: mol_type = genomic DNA
ttgcttctattgagctgtgcccagccgcccagtgacagaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 25 - DNA, 1735 bp, Homo sapiens
  source 1..1735: mol_type = genomic DNA
acagctgggagagcgcaggaaggcgccgggaaggaaagccagcccaccagcgtctgggtgggctgcgccgcgcggctggcggaccttcccgggttggagaggtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 26 - protein, 474 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQELRNEPANNQGEESAEIIPLNIIEQESSA

SEQ ID NO: 27 - protein, 508 aa, Homo sapiens
MQFSGEKISGQRDLQKSKMRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRKLTTIFPLNWKYRKALSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQELRNEPANNQGEESAEIIPLNIIEQESSA

SEQ ID NO: 28 - protein, 427 aa, Homo sapiens
MMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQELRNEPANNQGEESAEIIPLNIIEQESSA

SEQ ID NO: 29 - protein, 443 aa, Homo sapiens
MMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRKLTTIFPLNWKYRKALSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQELRNEPANNQGEESAEIIPLNIIEQESSA

SEQ ID NO: 30 - protein, 490 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRKLTTIFPLNWKYRKALSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQELRNEPANNQGEESAEIIPLNIIEQESSA

SEQ ID NO: 31 - protein, 490 aa, Homo sapiens; identical to SEQ ID NO: 30

SEQ ID NO: 32 - protein, 474 aa, Homo sapiens; identical to SEQ ID NO: 26

SEQ ID NO: 33 - protein, 474 aa, Homo sapiens; identical to SEQ ID NO: 26

SEQ ID NO: 34 - protein, 490 aa, Homo sapiens; identical to SEQ ID NO: 30

SEQ ID NO: 35 - protein, 474 aa, Homo sapiens; identical to SEQ ID NO: 26

SEQ ID NO: 36 - protein, 427 aa, Homo sapiens; identical to SEQ ID NO: 28

SEQ ID NO: 37 - protein, 443 aa, Homo sapiens; identical to SEQ ID NO: 29

SEQ ID NO: 38 - protein, 274 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRSLGCQRVTLHKNMFLTYILNSMIIIIHLVEVVPNGELVRRDPVSCKILHFFHQYMMACNYFWMLCEGIYLHTLIVVAVFTEKQRLRWYYLLGWAPAFHRD

SEQ ID NO: 39 - protein, 290 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRKLTTIFPLNWKYRKALSLGCQRVTLHKNMFLTYILNSMIIIIHLVEVVPNGELVRRDPVSCKILHFFHQYMMACNYFWMLCEGIYLHTLIVVAVFTEKQRLRWYYLLGWAPAFHRD

(19) United States
(12) Patent Application Publication
(10) Pub. No.: US 2024/0182973 A1
(43) Pub. Date: Jun. 6, 2024

(54) Treatment Of Obesity In Subjects Having Variant Nucleic Acid Molecules Encoding Calcitonin Receptor (CALCR)

(71) Applicant: Regeneron Pharmaceuticals, Inc., Tarrytown, NY (US)

(72) Inventors: Luca Andrea Lotta, Tarrytown, NY (US); Parsa Akbari, Tarrytown, NY (US); Manuel Allen Revez Ferreira, Tarrytown, NY (US); Aris Baras, Tarrytown, NY (US)

(21) Appl. No.: 18/582,159

(22) Filed: Feb. 20, 2024

Related U.S. Application Data
(63) Continuation of application No. 17/401,679, filed on Aug. 13, 2021, now Pat. No. 11,939,635.
(60) Provisional application No. 63/108,308, filed on Oct. 31, 2020.
(60) Provisional application No. 63/066,182, filed on Aug. 15, 2020.

(51) Int. Cl. C12Q 1/6883

(52) U.S. Cl. CPC C12Q 1/6883; C12Q 2600/118; C12Q 2600/156

(57) ABSTRACT

The present disclosure provides methods of treating subjects having obesity and/or increased body mass index (BMI), and methods of identifying subjects having an increased risk of developing obesity and/or BMI.

### REFERENCE TO SEQUENCE LISTING

This application includes a Sequence Listing submitted electronically as an XML file named 381204161SEQ, created on Feb. 8, 2024, with a size of 235,469 bytes. The Sequence Listing is incorporated herein by reference.

### FIELD

The present disclosure provides methods of treating a subject having a CALCR variant nucleic acid molecule encoding a CALCR predicted loss-of-function polypeptide with a therapeutic agent that treats or inhibits obesity, and methods of identifying subjects having an increased risk of developing obesity.

### BACKGROUND

Obesity and its cardio-metabolic complications, in particular type 2 diabetes and coronary artery disease, account for significant morbidity and mortality globally. There is a substantial unmet medical need for safe and effective weight loss approaches and treatments for obesity.

Lifestyle interventions on diet and physical activity are the first option for the management of obesity and overweight, but efficacy can be limited, and weight regain is common. Bariatric surgery can be highly effective for weight loss in severely obese or high-risk patients, but its use is limited by its invasive nature, cost, risk of perioperative adverse events including perioperative death. While a few therapeutic agents have demonstrated efficacy in weight-reduction, pharmacotherapy for the treatment of obesity is limited by the modest weight loss induced by most therapeutic agents, side effect profile of some agents, contraindications, low compliance, and barriers to treatment including underprescription.

Calcitonin Receptor (CALCR) is a G protein-coupled receptor that is highly expressed in the hypothalamus and other regions of the brain. The peptide hormones calcitonin and amylin are known ligands of CALCR.

### SUMMARY

The present disclosure provides methods of treating a subject with a therapeutic agent that treats or inhibits obesity and/or reduces BMI, wherein the subject has obesity and/or increased BMI, the methods comprising the steps of: determining whether the subject has a CALCR variant nucleic acid molecule encoding a CALCR predicted loss-of-function polypeptide by: obtaining or having obtained a biological sample from the subject; and performing or having performed a sequence analysis on the biological sample to determine if the subject has a genotype comprising the CALCR variant nucleic acid molecule; and administering or continuing to administer to the subject the therapeutic agent that treats or inhibits obesity and/or increased BMI in a standard dosage amount to a subject that is CALCR reference; or administering or continuing to administer to the subject the therapeutic agent that treats or inhibits obesity and/or increased BMI in an amount that is the same as or greater than a standard dosage amount to a subject that is heterozygous or homozygous for the CALCR variant nucleic acid molecule; wherein the presence of a genotype having the CALCR variant nucleic acid molecule encoding a CALCR predicted loss-of-function polypeptide indicates the subject has an increased risk of developing obesity and/or increased BMI.

The present disclosure also provides methods of identifying a subject having an increased risk of developing obesity and/or increased BMI, the methods comprising: determining or having determined the presence or absence of a CALCR variant nucleic acid molecule encoding a CALCR predicted loss-of-function polypeptide in a biological sample obtained from the subject; wherein: when the subject is CALCR reference, the subject does not have an increased risk of developing obesity and/or increased BMI; and when the subject is heterozygous or homozygous for a CALCR variant nucleic acid molecule, then the subject has an increased risk of developing obesity and/or increased BMI.

The present disclosure also provides therapeutic agents that treat or inhibit obesity and/or increased BMI for use in the treatment of obesity and/or increased BMI in a subject having: a CALCR variant genomic nucleic acid molecule encoding a CALCR predicted loss-of-function polypeptide; a CALCR variant mRNA molecule encoding a CALCR predicted loss-of-function polypeptide; or a CALCR variant cDNA molecule encoding a CALCR predicted loss-of-function polypeptide.

### BRIEF DESCRIPTION OF THE DRAWINGS

FIG. 1 shows positions of CALCR variants associated with BMI (p<0.05) relative to structural domains of the CALCR protein; pink vertical lines indicate variants associated with higher BMI; blue vertical lines indicate variants associated with lower BMI; variants indicated in the black boxes are: 1) S18P, 2) V621, 3) R92C, 4) K125fs, 5) 1178V, 6) S209N, 7) R355Q, 8) F390V, 9) V3921, 10) A422V, 11) R432C, 12) S456F, 13) R461fs, 14) L481P, and 15) N487fs; the amino acid sequences shown are several isoforms of proteins resulting from human CALCR splice variants including, top to bottom, H. sapiens NCBI NP_001158209.2, H. sapiens NCBI NP_001158210.1 (Equ. P30988-2), H. sapiens NCBI NP_001733.1 (Equ. P30988-2), H. sapiens Uniprot P30988-1, H. sapiens Uniprot P30988-2, H. sapiens Uniprot P30988-3, H. sapiens Uniprot P30988-4, H. sapiens Uniprot P30988-5, and H. sapiens Uniprot P30988-6; the black underlined portions represent transmembrane domains, the green underlined portions represent the extracellular domain, and the red underlined portions represent the intracellular domain.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

It has been observed in accordance with the present disclosure that a gene burden of CALCR variant nucleic acid molecules (whether these variations are homozygous or heterozygous in a particular subject) encoding CALCR loss-of-function polypeptides is associated with an increased risk of developing obesity and/or elevated BMI. It is believed that loss-of-function variants in the CALCR gene or protein have not been associated with obesity or elevated BMI in genome-wide or exome-wide association studies. Therefore, subjects that are homozygous or heterozygous for a CALCR variant nucleic acid molecule encoding a CALCR predicted loss-of-function polypeptide that associate with obesity and/or elevated BMI may be treated such that obesity and/or elevated BMI is inhibited, the symptoms thereof are reduced, and/or development of symptoms is repressed. It is also believed that such subjects having obesity and/or elevated BMI may be treated with therapeutic agents that treat or inhibit obesity and/or increased BMI.

For purposes of the present disclosure, any particular subject, such as a human, can be categorized as having one of three CALCR genotypes: i) CALCR reference; ii) heterozygous for a CALCR variant nucleic acid molecule encoding a CALCR predicted loss-of-function polypeptide; or iii) homozygous for a CALCR variant nucleic acid molecule encoding a CALCR predicted loss-of-function polypeptide. A subject is CALCR reference when the subject does not have a copy of a CALCR variant nucleic acid molecule encoding a CALCR predicted loss-of-function polypeptide. A subject is heterozygous for a CALCR variant nucleic acid molecule encoding a CALCR predicted loss-of-function polypeptide when the subject has a single copy of a CALCR variant nucleic acid molecule encoding a CALCR predicted loss-of-function polypeptide. A CALCR variant nucleic acid molecule is any nucleic acid molecule (such as, a genomic nucleic acid molecule, an mRNA molecule, or a cDNA molecule) encoding a CALCR polypeptide having a partial loss-of-function, a complete loss-of-function, a predicted partial loss-of-function, or a predicted complete loss-of-function. A subject who has a CALCR polypeptide having a partial loss-of-function (or predicted partial loss-of-function) is hypomorphic for CALCR. A subject is homozygous for a CALCR variant nucleic acid molecule encoding a CALCR predicted loss-of-function polypeptide when the subject has two copies (same or different) of a CALCR variant nucleic acid molecule encoding a CALCR predicted loss-of-function polypeptide.

For subjects that are genotyped or determined to be heterozygous or homozygous for a CALCR variant nucleic acid molecule encoding a CALCR predicted loss-of-function polypeptide, such subjects have an increased risk of developing obesity, such as increased BMI, type 1 obesity, type 2 obesity, or type 3 obesity, or have an increased risk of developing increased BMI. For subjects that are genotyped or determined to be heterozygous or homozygous for a CALCR variant nucleic acid molecule encoding a CALCR predicted loss-of-function polypeptide, such subjects can be treated with an agent effective to treat obesity, such as increased BMI, type 1 obesity, type 2 obesity, or type 3 obesity, and/or with an agent effective to treat increased BMI.

In any of the embodiments described herein, the CALCR variant nucleic acid molecule can be any nucleic acid molecule (such as, for example, genomic nucleic acid molecule, mRNA molecule, or cDNA molecule) encoding a CALCR polypeptide having a partial loss-of-function, a complete loss-of-function, a predicted partial loss-of-function, or a predicted complete loss-of-function. In some embodiments, the CALCR variant nucleic acid molecule is associated with a reduced in vitro response to calcitonin, amylin, GCRP, adrenomedullin, or other ligand of CALCR compared with reference CALCR. In some embodiments, the CALCR variant nucleic acid molecule is a CALCR nucleic acid molecule that results or is predicted to result in a premature truncation of a CALCR polypeptide compared to the human reference genome sequence. In some embodiments, the CALCR variant nucleic acid molecule is a variant that is predicted to be damaging by in vitro prediction algorithms such as Polyphen, SIFT, or similar algorithms. In some embodiments, the CALCR variant nucleic acid molecule is a variant that causes or is predicted to cause a nonsynonymous amino-acid substitution in a CALCR polypeptide and whose allele frequency is less than 1/1,000 alleles in the population from which the subject is selected. In some embodiments, the CALCR variant nucleic acid molecule is any rare missense variant (allele frequency <0.1%; or 1 in 1,000 alleles), or any splice-site, stop-gain, start-loss, stop-loss, frameshift, or in-frame indel, or other frameshift CALCR variant. In some embodiments, the subject has one or two of the following CALCR variant nucleic acid molecules: 7:93426412:C:A, 7:93426422:GT:G, 7:93426434:AT:A, 7:93426501:CG:C, 7:93426512:G:GT, 7:93426517:A:AG, 7:93426559:G:A, 7:93426561:G:GC, 7:93426564:C:T, 7:93434252:C:T, 7:93434288:AG:A, 7:93434289:G:GA, 7:93436171:C:A, 7:93436171:C:T, 7:93436171:CTGCAAATATACGG:C, 7:93438056:TCAC:T, 7:93438059:C:G, 7:93438209:C:T, 7:93443603:C:A, 7:93443607:AGCCCAAGAGATAATACC:A, 7:93443648:AAC:A, 7:93443743:AATCTT:A, 7:93443758:C:T, 7:93460832:G:A, 7:93460949:T:C, 7:93462060:T:TA, 7:93462112:T:C, 7:93468789:G:T, 7:93472373:A:G, 7:93472384:C:CT, 7:93472420:C:T, 7:93472428:G:A, 7:93472446:T:TA, 7:93472483:CT:C, 7:93477556:A:C, 7:93477557:C:A, 7:93477628:CCAGCA:C, 7:93477660:AAT:A, 7:93479387:G:A, 7:93479426:G:A, 7:93479450:T:TG, 7:93486979:CATTTTTG:C, 7:93487001:CTT:C, 7:93487008:C:T, 7:93495902:G:T, 7:93495903:C:T, 7:93495930:A:G, 7:93495976:T:A, or 7:93495976:T:C.

In some embodiments, the subject has one or two of the following CALCR variant nucleic acid molecules: 7:93426422:GT:G, 7:93426441:A:G, 7:93426501:CG:C, 7:93426516:G:A, 7:93426571:G:A, 7:93434281:G:A, 7:93436029:C:T, 7:93436035:A:C, 7:93436139:C:T, 7:93460945:C:T, 7:93468758:T:C, 7:93472483:CT:C, 7:93477654:G:A, 7:93479432:C:T, or 7:93486987:A:G. All variants are associated with increased BMI except 7:93426441:A:G and 7:93479432:C:T, which are associated with decreased BMI.

In some embodiments, the subject has one or two of the following variant CALCR polypeptides: S18P, V621, R92C, K125fs, 1178V, S209N, R355Q, F390V, V3921, A422V, R432C, S456F, R461fs, L481P, or N487fs. All variants are associated with increased BMI except V621 and L481P, which are associated with decreased BMI. Numerous human CALCR splice variants lead to several isoforms of proteins, which can be designated by, for example: H. sapiens NCBI NP_001158209.2, H. sapiens NCBI NP_001158210.1 (Equ. P30988-2), H. sapiens NCBI NP_001733.1 (Equ. P30988-2), H. sapiens Uniprot P30988-1, H. sapiens Uniprot P30988-2, H. sapiens Uniprot P30988-3, H. sapiens Uniprot P30988-4, H. sapiens Uniprot P30988-5, and H. sapiens Uniprot P30988-6.

In some embodiments, the subject has one or two of the following CALCR variant nucleic acid molecules: 7:93426364:A:G, 7:93426378:A:G, 7:93426384:T:G, 7:93426385:T:A, 7:93426398:C:G, 7:93426408:T:C, 7:93426412:C:A, 7:93426412:C:T, 7:93426412:C:G, 7:93426414:C:T, 7:93426422:GT: G, 7:93426434:AT:A, 7:93426451:G:A, 7:93426456:A:G, 7:93426469:T:A, 7:93426475:C:A, 7:93426481:C:A, 7:93426490:C:T, 7:93426496:C:A, 7:93426501:CG:C, 7:93426501:C:T, 7:93426502:G:A, 7:93426505:C:G, 7:93426511:G:T, 7:93426511:G:C, 7:93426511:G:A, 7:93426512:G:GT, 7:93426517:A:AG, 7:93426526:T:TC, 7:93426528:C:T, 7:93426530:C:G, 7:93426531:C:G, 7:93426534:C:T, 7:93426534:C:G, 7:93426535:G:C, 7:93426538:G:T, 7:93426543:C:T, 7:93426545:C:G, 7:93426546:T:C, 7:93426561:G:GC, 7:93426563:C:G, 7:93426564:C:T, 7:93426565:A:G, 7:93426571:G:A, 7:93426572:C:A, 7:93426577:C:T, 7:93426585:T:C, 7:93426589:C:T, 7:93434252:C:T, 7:93434261:T:G, 7:93434262:G:C, 7:93434267:A:G, 7:93434269:C:G, 7:93434272:T:A, 7:93434281:G:A, 7:93434285:C:T, 7:93434287:A:G, 7:93434288:AG:A, 7:93434289:GA:G, 7:93435951:C:T, 7:93435971:T:C, 7:93435971:T:A, 7:93435978:C:T, 7:93435980:T:C, 7:93435983:T:A, 7:93435987:A:G, 7:93435988:T:C, 7:93436007:T:C, 7:93436011:A:G, 7:93436023:G:C, 7:93436029:C:T, 7:93436035:A:C, 7:93436059:G:A, 7:93436062:T:C, 7:93436065:T:C, 7:93436091:A:G, 7:93436091:A:C, 7:93436103:G:A, 7:93436103:G:T, 7:93436139:C:G, 7:93436139:C:T, 7:93436140:G:A, 7:93436142:A:T, 7:93436151:A:G, 7:93436166:T:C, 7:93436169:A:G, 7:93436171:C:A, 7:93438056:TCAC:T, 7:93438059:C:G, 7:93438070:G:A, 7:93438070:G:T, 7:93438072:C:A, 7:93438073:A:G, 7:93438074:T:C, 7:93438077:C:G, 7:93438080:G:T, 7:93438080:G:A, 7:93438103:T:C, 7:93438106:G:A, 7:93438115:C:T, 7:93438116:T:C, 7:93438125:A:G, 7:93438127:C:T, 7:93438209:C:T, 7:93438226:C:T, 7:93438229:C:T, 7:93438238:T:G, 7:93438243:T:A, 7:93438247:T:A, 7:93438247:T:C, 7:93438252:G:A, 7:93438256:G:T, 7:93438264:G:A, 7:93438266:G:C, 7:93438270:C:A, 7:93443603:C:A, 7:93443605:C:G, 7:93443607:AGCCCAAGAGATAATACC:A, 7:93443611:CAA:C, 7:93443618:T:C, 7:93443621:T:C, 7:93443623:C:T, 7:93443624:C:A, 7:93443625:A:G, 7:93443628:G:A, 7:93443634:G:A, 7:93443637:G:C, 7:93443642:T:C, 7:93443648:AAC:A, 7:93443652:C:T, 7:93443658:C:T, 7:93443678:T:C, 7:93443681:A:T, 7:93443682:T:G, 7:93443694:G:A, 7:93443696:A:G, 7:93443703:A:G, 7:93443705:T:C, 7:93443708:T:G, 7:93443711:C:G, 7:93443718:T:C, 7:93443719:C:G, 7:93443727:G:C, 7:93443727:G:T, 7:93443729:T:A, 7:93443733:A:G, 7:93443739:G:C, 7:93443739:G:T, 7:93443743:AATCTT:A, 7:93443746:C:G, 7:93443746:C:A, 7:93443758:C:T, 7:93460819:AC:A, 7:93460822:G:A, 7:93460822:G:T, 7:93460826:C:A, 7:93460831:C:A, 7:93460832:G:A, 7:93460835:C:G, 7:93460835:C:T, 7:93460838:G:A, 7:93460842:TC:T, 7:93460845:A:C, 7:93460855:A:C, 7:93460856:C:T, 7:93460856:C:A, 7:93460867:T:C, 7:93460888:T:C, 7:93460891:A:G, 7:93460900:G:T, 7:93460909:A:G, 7:93460919:G:A, 7:93460924:GT:G, 7:93460924:G:T, 7:93460924:G:C, 7:93460931:T:C, 7:93460936:C:T, 7:93460943:G:A, 7:93460945:C:T, 7:93460949:T:A, 7:93460949:T:C, 7:93462060:T:TA, 7:93462062:C:T, 7:93462066:G:A, 7:93462066:G:T, 7:93462067:C:A, 7:93462086:C:G, 7:93462087:A:G, 7:93462090:G:T, 7:93462090:G:A, 7:93462091:GA:G, 7:93462091:G:A, 7:93462097:T:A, 7:93462102:G:T, 7:93462103:T:C, 7:93462104:C:G, 7:93462112:T:C, 7:93468714:C:A, 7:93468717:G:C, 7:93468722:A:C, 7:93468731:TC:T, 7:93468753:G:T, 7:93468758:T:C, 7:93468759:TG:T, 7:93468762:C:T, 7:93468764:A:G, 7:93468769:T:C, 7:93468770:G:GACCCACA, 7:93468772:C:G, 7:93468775:A:G, 7:93468781:G:C, 7:93468787:T:G, 7:93468789:G:T, 7:93468799:T:C, 7:93472374:C:T, 7:93472377:T:C, 7:93472379:A:G, 7:93472381:T:A, 7:93472383:T:C, 7:93472384:C:CT, 7:93472392:T:G, 7:93472400:T:C, 7:93472401:T:G, 7:93472402:G:T, 7:93472412:T:C, 7:93472418:G:T, 7:93472418:GA:G, 7:93472418:G:C, 7:93472424:G:T, 7:93472427:C:T, 7:93472428:G:A, 7:93472432:G:C, 7:93472437:C:T, 7:93472446:T:TA, 7:93472456:AC:A, 7:93472470:A:G, 7:93472473:A:G, 7:93472479:TA:T, 7:93472482:C:T, 7:93472483:CT:C, 7:93472486:T:G, 7:93472488:C:T, 7:93472489:T:C, 7:93477556:A:C, 7:93477557:C:G, 7:93477567:CA:C, 7:93477575:G:A, 7:93477584:T:C, 7:93477587:G:T, 7:93477595:C:A, 7:93477602:G:A, 7:93477602:G:T, 7:93477611:C:A, 7:93477612:C:G, 7:93477617:G:A, 7:93477617:G:T, 7:93477617:G:C, 7:93477620:G:A, 7:93477622:G:C, 7:93477622:G:T, 7:93477623:T:C, 7:93477628:CCAGCA:C, 7:93477633:A:C, 7:93477633:A:G, 7:93477638:C:G, 7:93477639:A:G, 7:93477644:T:C, 7:93477650:G:A, 7:93477650:G:T, 7:93477653:C:T, 7:93477654:G:A, 7:93477656:T:C, 7:93477660:A:G, 7:93477661:A:T, 7:93477668:C:A, 7:93479372:G:A, 7:93479381:G:A, 7:93479383:A:T, 7:93479386:C:T, 7:93479386:C:G, 7:93479387:G:A, 7:93479393:A:T, 7:93479410:T:C, 7:93479413:A:G, 7:93479422:T:C, 7:93479425:C:T, 7:93479426:G:A, 7:93479450:T:TG, 7:93479453:G:T, 7:93479470:G:T, 7:93486934:TAG:T, 7:93486957:A:T, 7:93486979:CATTTTTG:C, 7:93486986:G:A, 7:93486986:G:T, 7:93486995:A:G, 7:93487005:G:T, 7:93487007:C:T, 7:93487008:C:T, 7:93495902:G:A, 7:93495902:G:T, 7:93495903:C:T, 7:93495929:C:T, 7:93495930:A:G, 7:93495976:T:A, 7:93495976:T:C, 7:93426360:G:T, 7:93426391:G:T, 7:93426393:A:T, 7:93426399:T:C, 7:93426433:C:G, 7:93426459:T:A, 7:93426465:G:C, 7:93426483:G:A, 7:93426492:G:T, 7:93426493:C:T, 7:93426519:G:T, 7:93426535:G:T, 7:93426559:G:A, 7:93426570:C:T, 7:93426582:G:T, 7:93435953:T:A, 7:93436006:G:T, 7:93436032:C:G, 7:93436068:T:A, 7:93436113:T:A, 7:93436127:G:A, 7:93438085:T:C, 7:93438095:A:C, 7:93438101:A:T, 7:93438109:T:A, 7:93438112:A:G, 7:93438124:C:T, 7:93438255:G:A, 7:93438259:C:T, 7:93443622:A:G, 7:93443624:C:G, 7:93443657:A:G, 7:93443682:T:A, 7:93443720:A:G, 7:93443734:A:T, 7:93443753:C:G, 7:93460846:T:C, 7:93460906:A:G, 7:93460918:T:C, 7:93460929:C:A, 7:93460930:C:T, 7:93460933:T:C, 7:93460939:C:T, 7:93462086:CA:C, 7:93468759:T:C, 7:93468769:T:G, 7:93468781:G:A, 7:93468787:T:C, 7:93472373:A:G, 7:93472397:G:C, 7:93472415:T:C, 7:93472420:C:T, 7:93472458:C:A, 7:93472472:T:C, 7:93472482:C:A, 7:93477557:C:A, 7:93477566:T:A, 7:93477590:C:T, 7:93477623:T:G, 7:93477626:T:G, 7:93477632:C:A, 7:93477637:C:G, 7:93477644:T:A, 7:93477654:G:C, 7:93477662:T:C, 7:93479371:G:A, 7:93479404:T:C, 7:93479426:G:C, 7:93479456:C:T, 7:93486944:A:G, 7:93487001:CTT:C, 7:93487005:G:A, 7:93495910:T:A, 7:93426376:C:T, 7:93426588:A:C, 7:93434289:G:GA, 7:93435991:C:A, 7:93436005:T:C, 7:93436053:G:A, 7:93436079:G:A, 7:93436110:G:A, 7:93436171:CTGCAAATATACGG:C, 7:93436171:C:T, 7:93438089:T:C, 7:93438092:T:C, 7:93443643:C:T, 7:93443666:A:C, 7:93443750:C:T, 7:93460835:C:A, 7:93460897:T:C, 7:93468782:C:T, 7:93472424:G:A, 7:93472463:T:C, 7:93477599:T:C, 7:93477653:C:A, or 7:93477660:AAT:A.

In any of the embodiments described herein, the CALCR variant nucleic acid molecules have variations at the indicated positions of chromosome 7 using the nucleotide sequence of the CALCR reference genomic nucleic acid molecule (SEQ ID NO:1; ENST00000426151.6 encompassing chr7:93,424,539-93,574,730 in the GRCh38/hg38 human genome assembly; ENSG00000004948.15) as a reference sequence. An additional sequence (ENST00000360249.8 encompassing chr7:93,426,310-93,487,013 in GRCh38/hg38 human genome assembly; ENSG00000004948.15) can also be used as the CALCR reference sequence.

In any of the embodiments described herein, the CALCR variant nucleic acid molecules encoding variations in the protein sequence can include nucleotides at the indicated positions of chromosome 7 using the nucleotide sequence of the CALCR reference genomic nucleic acid molecule (SEQ ID NO:1; ENST00000426151.6 encompassing chr7:93,424,539-93,574,730 in the GRCh38/hg38 human genome assembly; ENSG00000004948.15) as a reference sequence.

Any one or more (i.e., any combination) of the CALCR variants recited herein can be used within any of the methods described herein to determine whether a subject has an increased risk of developing obesity and/or increased BMI. The combinations of particular variants can form a mask used for statistical analysis of the particular correlation of CALCR and increased obesity/BMI risk.

In any of the embodiments described herein, the CALCR predicted loss-of-function polypeptide can be any CALCR polypeptide having a partial loss-of-function, a complete loss-of-function, a predicted partial loss-of-function, or a predicted complete loss-of-function.

In any of the embodiments described herein, the obesity is type 1 obesity, type 2 obesity, or type 3 obesity. In any of the embodiments described herein, the obesity is type 1 obesity. In any of the embodiments described herein, the obesity is type 2 obesity. In any of the embodiments described herein, the obesity is type 3 obesity. In any of the embodiments described herein, the subject has increased BMI.

Symptoms of obesity include, but are not limited to, excess body fat accumulation (particularly around the waist), breathlessness, increased sweating, snoring, inability to cope with sudden physical activity, feeling very tired every day, back and joint pains, skin problems (from moisture accumulating in the folds of skin).

The present disclosure provides methods of treating a subject with a therapeutic agent that treats or inhibits obesity and/or reduces BMI. In these methods, the subject has obesity and/or increased BMI. The methods comprise the steps of determining whether the subject has a CALCR variant nucleic acid molecule encoding a CALCR predicted loss-of-function polypeptide. In these methods, such determination step comprises obtaining or having obtained a biological sample from the subject. In these methods, such determination step also comprises performing or having performed a sequence analysis on the biological sample to determine if the subject has a genotype comprising the CALCR variant nucleic acid molecule. When the subject is CALCR reference, then the methods further comprise administering or continuing to administer to the subject the therapeutic agent that treats or inhibits obesity and/or increased BMI in a standard dosage amount. When the subject is heterozygous or homozygous for the CALCR variant nucleic acid molecule encoding a CALCR predicted loss-of-function polypeptide, then the methods further comprise administering or continuing to administer to the subject the therapeutic agent that treats or inhibits obesity and/or increased BMI in an amount that is the same as or greater than a standard dosage amount. The presence of a genotype having the CALCR variant nucleic acid molecule indicates the subject has an increased risk of developing obesity and/or increased BMI.

In some embodiments, the subject has obesity. In some embodiments, the subject has increased BMI. In some embodiments, the subject is CALCR reference. In some embodiments, the subject is heterozygous for a CALCR variant nucleic acid molecule encoding a CALCR predicted loss-of-function polypeptide. In some embodiments, the subject is homozygous for a CALCR variant nucleic acid molecule encoding a CALCR predicted loss-of-function polypeptide.

In some embodiments, the methods of treatment further comprise detecting the presence or absence of a CALCR variant nucleic acid molecule encoding a CALCR predicted loss-of-function polypeptide in a biological sample from the subject. As used throughout the present disclosure, a “CALCR variant nucleic acid molecule” is any nucleic acid molecule (such as, for example, genomic nucleic acid molecule, mRNA molecule, or cDNA molecule) encoding a CALCR polypeptide having a partial loss-of-function, a complete loss-of-function, a predicted partial loss-of-function, or a predicted complete loss-of-function. In some embodiments, the CALCR variant nucleic acid molecule is associated with a reduced in vitro response to calcitonin, amylin, GCRP, adrenomedullin, or other ligand of CALCR compared with reference CALCR. In some embodiments, the CALCR variant nucleic acid molecule results or is predicted to result in a premature truncation of a CALCR polypeptide compared to the human reference genome sequence. In some embodiments, the CALCR variant nucleic acid molecule is a variant that is predicted to be damaging by in vitro prediction algorithms such as Polyphen, SIFT, or similar algorithms. In some embodiments, the CALCR variant nucleic acid molecule is a variant that causes or is predicted to cause a nonsynonymous amino-acid substitution in CALCR and whose allele frequency is less than 1/1,000 alleles in the population from which the subject is selected. In some embodiments, the CALCR variant nucleic acid molecule is any rare missense variant (allele frequency <0.1%; or 1 in 1,000 alleles), or any splice-site, stop-gain, start-loss, stop-loss, frameshift, or in-frame indel, or other frameshift CALCR variant.

Detecting the presence or absence of a CALCR variant nucleic acid molecule encoding a CALCR predicted loss-of-function polypeptide in a biological sample from a subject and/or determining whether a subject has a CALCR variant nucleic acid molecule encoding a CALCR predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of treating a subject with a therapeutic agent that treats or inhibits obesity and/or increased BMI, wherein the subject has obesity and/or increased BMI. In some embodiments, the methods comprise determining whether the subject has a predicted loss-of-function CALCR polypeptide by obtaining or having obtained a biological sample from the subject, and performing or having performed an assay on the biological sample to determine if the subject has a CALCR predicted loss-of-function polypeptide. When the subject does not have a CALCR predicted loss-of-function polypeptide, the therapeutic agent that treats or inhibits obesity and/or increased BMI is administered or continued to be administered to the subject in a standard dosage amount. When the subject has a CALCR predicted loss-of-function polypeptide, the therapeutic agent that treats or inhibits obesity and/or increased BMI is administered or continued to be administered to the subject in an amount that is the same as or greater than a standard dosage amount. The presence of a CALCR predicted loss-of-function polypeptide indicates the subject has an increased risk of developing obesity and/or increased BMI. In some embodiments, the subject has a CALCR predicted loss-of-function polypeptide. In some embodiments, the subject does not have a CALCR predicted loss-of-function polypeptide.

Detecting the presence or absence of a CALCR predicted loss-of-function polypeptide in a biological sample from a subject and/or determining whether a subject has a CALCR predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of therapeutic agents that treat or inhibit obesity and/or increased BMI also include, but are not limited to, sibutramine, orlistat, phentermine, topiramate, lorcaserin, bupropion, Contrave® (naltrexone), Saxenda® (liraglutide), phentermine, diethylpropion, bupropion, metformin, Symlin® (pramlintide), topiramate, zonisamide, Ozempic® (semaglutide), Tirzepatide® (LY3298176), miacalcin, amylin, Myalept® (metreleptin), Qsymia® (phentermine-topiramate), Byetta® (exenatide), Trulicity® (dulaglutide), AM833, davalintide, KBP-088, GCRP, adrenomedullin, leptin, PYY, cholecystokinin, PYY1875, LA-GDF15, PF-06882961, PF-07081532, cotadutide (MEDI0382), efinopegdutide (HM12525A), pegbelfermin, BIO89-100, efruxifermin, YH-25724, BFKB-8488A, NGM-313, NGM-282, lanifibranor, aldafermin, resmetirom, seladelpar, Ocaliva® (obeticholic acid), AMG 171, NN9215, JNJ-9090, NGM395, NGM120, AV-380, efpeglenatide, AKR-001, Aldafermin (NGM282), RG7992 (BFKB-8488A), BIO89-100, LLF-580, NGM-313, NN-9500, TSLB-1344, YH-25724, LY2405319, PF-05231023, Fc-FGF21(RGE), BMS-986036, TBRIA® (calcitonin), or oxyntomodulin, or any combination thereof. In some embodiments, the therapeutic agent that treats or inhibits obesity and/or increased BMI is sibutramine, orlistat, phentermine, topiramate, lorcaserin, bupropion, naltrexone, liraglutide, phentermine, diethylpropion, bupropion, metformin, pramlintide, topiramate, zonisamide, semaglutide, LY3298176, miacalcin, amylin, metreleptin, phentermine-topiramate, exenatide, dulaglutide, AM833, davalintide, KBP-088, GCRP, adrenomedullin, leptin, PYY, cholecystokinin, PYY1875, LA-GDF15, PF-06882961, PF-07081532, cotadutide (MEDI0382), efinopegdutide (HM12525A), pegbelfermin, BIO89-100, efruxifermin, YH-25724, BFKB-8488A, NGM-313, NGM-282, lanifibranor, aldafermin, resmetirom, seladelpar, obeticholic acid, AMG 171, NN9215, JNJ-9090, NGM395, NGM120, AV-380, efpeglenatide, AKR-001, Aldafermin (NGM282), RG7992 (BFKB-8488A), BIO89-100, LLF-580, NGM-313, NN-9500, TSLB-1344, YH-25724, LY2405319, PF-05231023, Fc-FGF21(RGE), BMS-986036, calcitonin, or oxyntomodulin, or any combination thereof.

In some embodiments, the therapeutic agent that treats or inhibits obesity and/or reduces BMI is a melanocortin 4 receptor (MC4R) agonist. In some embodiments, the MC4R agonist comprises a protein, a peptide, a nucleic acid molecule, or a small molecule. In some embodiments, the protein is a peptide analog of MC4R. In some embodiments, the peptide is setmelanotide. In some embodiments, the therapeutic agent that treats or inhibits obesity and/or reduces BMI is a combination of setmelanotide and one or more of sibutramine, orlistat, phentermine, lorcaserin, naltrexone, liraglutide, diethylpropion, bupropion, metformin, pramlintide, topiramate, and zonisamide. In some embodiments, the MC4R agonist is a peptide comprising the amino acid sequence His-Phe-Arg-Trp. In some embodiments, the small molecule is 1,2,3R,4-tetrahydroisoquinoline-3-carboxylic acid. In some embodiments, the MC4R agonist is ALB-127158(a).

In some embodiments, the therapeutic agent that treats or inhibits obesity and/or reduces BMI is a CALCR agonist. In some embodiments, the CALCR agonist is calcitonin, amylin, GCRP, adrenomedullin, Symlin® (pramlintide), miacalcin, AM833, davalintide, KBP-088, leptin, PYY, or cholecystokinin, or any combination thereof. In some embodiments, the CALCR agonist is calcitonin, amylin, GCRP, adrenomedullin, pramlintide, miacalcin, AM833, davalintide, KBP-088, leptin, PYY, or cholecystokinin, or any combination thereof. In some embodiments, the CALCR agonist is calcitonin or amylin. In some embodiments, the CALCR agonist is GCRP or adrenomedullin. In some embodiments, the CALCR agonist is a synthetic or non-synthetic CALCR agonist.

In some embodiments, the therapeutic agents that treat or inhibit obesity and/or increased BMI can be administered to subjects with obesity but without a CALCR variant nucleic acid molecule or CALCR predicted loss-of-function polypeptide. In such embodiments, a goal would be to exploit the CALCR-mediated anti-obesity properties in subjects with intact CALCR signaling. In some embodiments, the therapeutic agents that treat or inhibit obesity and/or increased BMI can be administered to subjects with obesity and having the CALCR variant nucleic acid molecule or CALCR predicted loss-of-function polypeptide. In such embodiments, a goal would be to enhance CALCR-mediated anti-obesity properties in subjects with a relative deficiency in CALCR signaling due to their genotype.

In some embodiments, the dose of the therapeutic agents that treat or inhibit obesity and/or increased BMI can be increased by about 10%, by about 20%, by about 30%, by about 40%, by about 50%, by about 60%, by about 70%, by about 80%, or by about 90% for patients or human subjects that are heterozygous or homozygous for a CALCR variant nucleic acid molecule encoding a CALCR predicted loss-of-function polypeptide (i.e., a greater amount than the standard dosage amount) compared to subjects that are CALCR reference (who may receive a standard dosage amount). In some embodiments, the dose of the therapeutic agents that treat or inhibit obesity and/or increased BMI can be increased by about 10%, by about 20%, by about 30%, by about 40%, or by about 50%. In addition, the dose of therapeutic agents that treat or inhibit obesity and/or increased BMI in subjects that are heterozygous or homozygous for a CALCR variant nucleic acid molecule encoding a CALCR predicted loss-of-function polypeptide can be administered more frequently compared to subjects that are CALCR reference.

In some embodiments, the dose of the therapeutic agents that treat or inhibit obesity and/or increased BMI can be increased by about 10%, by about 20%, by about 30%, by about 40%, by about 50%, by about 60%, by about 70%, by about 80%, or by about 90% for subjects that are homozygous for a CALCR variant nucleic acid molecule encoding a CALCR predicted loss-of-function polypeptide compared to subjects that are heterozygous for a CALCR variant nucleic acid molecule encoding a CALCR predicted loss-of-function polypeptide. In some embodiments, the dose of the therapeutic agents that treat or inhibit obesity and/or increased BMI can be increased by about 10%, by about 20%, by about 30%, by about 40%, or by about 50%. In addition, the dose of therapeutic agents that treat or inhibit obesity and/or increased BMI in subjects that are homozygous for a CALCR variant nucleic acid molecule encoding a CALCR predicted loss-of-function polypeptide can be administered more frequently compared to subjects that are heterozygous for a CALCR variant nucleic acid molecule encoding a CALCR predicted loss-of-function polypeptide. Administration of the therapeutic agents that treat or inhibit obesity and/or increased BMI can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a patient can receive therapy for a prolonged period of time such as, for example, 6 months, 1 year, or more.

Administration of the therapeutic agents that treat or inhibit obesity and/or increased BMI can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries.

The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in obesity and/or increased BMI, a decrease/reduction in the severity of obesity and/or increased BMI (such as, for example, a reduction or inhibition of development of obesity and/or increased BMI), a decrease/reduction in symptoms and obesity-related effects, delaying the onset of symptoms and obesity-related effects, reducing the severity of symptoms of obesity-related effects, reducing the number of symptoms and obesity-related effects, reducing the latency of symptoms and obesity-related effects, an amelioration of symptoms and obesity-related effects, reducing secondary symptoms, reducing secondary infections, preventing relapse to obesity and/or increased BMI, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of obesity development/progression (such as, for example, a complete or partial avoidance/inhibition or a delay), and an increased survival time of the affected host animal, following administration of a therapeutic protocol. Treatment of obesity and/or increased BMI encompasses the treatment of patients already diagnosed as having any form of obesity and/or increased BMI at any clinical stage or manifestation, the delay of the onset or evolution or aggravation or deterioration of the symptoms or signs of obesity and/or increased BMI, and/or preventing and/or reducing the severity of obesity and/or increased BMI.

The present disclosure also provides methods of identifying a subject having an increased risk of developing obesity and/or increased BMI. These methods comprise determining or having determined the presence or absence of a CALCR variant nucleic acid molecule encoding a CALCR predicted loss-of-function polypeptide in a biological sample obtained from the subject. When the subject is CALCR reference, the subject does not have an increased risk of developing obesity and/or increased BMI. When the subject is heterozygous or homozygous for a CALCR variant nucleic acid molecule encoding a CALCR predicted loss-of-function polypeptide, the subject has an increased risk of developing obesity and/or increased BMI.

In some embodiments, the subject has obesity. In some embodiments, the subject has increased BMI. In some embodiments, the subject is CALCR reference. In some embodiments, the subject is heterozygous for a CALCR variant nucleic acid molecule encoding a CALCR predicted loss-of-function polypeptide. In some embodiments, the subject is homozygous for a CALCR variant nucleic acid molecule encoding a CALCR predicted loss-of-function polypeptide.

Determining whether a subject has a CALCR variant nucleic acid molecule encoding a CALCR predicted loss-of-function polypeptide in a biological sample from a subject and/or determining whether a subject comprises a CALCR variant nucleic acid molecule encoding a CALCR predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing obesity and/or increased BMI, the subject is further treated with a therapeutic agent that treats or inhibits obesity and/or increased BMI, as described herein. In some embodiments, when the subject is heterozygous or homozygous for CALCR variant nucleic acid molecule encoding a CALCR predicted loss-of-function polypeptide, the subject is administered the therapeutic agent that treats or inhibits obesity and/or increased BMI in a dosage amount that is the same as or greater than a standard dosage amount. In some embodiments, when the subject is homozygous for a CALCR variant nucleic acid molecule encoding a CALCR predicted loss-of-function polypeptide, the subject is administered the therapeutic agent that treats or inhibits obesity and/or increased BMI in a dosage amount that is the same as or greater than the dosage amount administered to a subject that is heterozygous for a CALCR variant nucleic acid molecule encoding a CALCR predicted loss-of-function polypeptide. In some embodiments, the subject is CALCR reference. In some embodiments, the subject is heterozygous for a CALCR variant nucleic acid molecule encoding a CALCR predicted loss-of-function polypeptide. In some embodiments, the subject is homozygous for a CALCR variant nucleic acid molecule encoding a CALCR predicted loss-of-function polypeptide.

In some embodiments, the methods can further comprise determining the subject's gene burden of having CALCR variant nucleic acid molecule (genomic, mRNA, or cDNA) encoding a CALCR predicted loss-of-function polypeptide associated with an increased risk of developing obesity and/or increased BMI, and/or CALCR predicted loss-of-function polypeptides associated with an increased risk of developing obesity and/or incrased BMI. The gene burden is the aggregate of all variants or rare variants in the CALCR gene, which can be carried out in an association analysis with obesity and/or increased BMI. In some embodiments, the subject is homozygous for one or more CALCR variant nucleic acid molecules encoding a CALCR predicted loss-of-function polypeptide associated with an increased risk of developing obesity and/or increased BMI. In some embodiments, the subject is heterozygous for one or more CALCR variant nucleic acid molecules encoding a CALCR predicted loss-of-function polypeptide associated with an increased risk of developing obesity and/or increased BMI. The result of the association analysis suggests that rare loss-of-function and missense variants of CALCR are associated with increased risk of obesity and increased BMI.

In some embodiments, the subject's gene burden of having any one or more CALCR variant nucleic acid molecules encoding a CALCR predicted loss-of-function polypeptide represents a weighted sum of a plurality of any of the CALCR variant nucleic acid molecules encoding a CALCR predicted loss-of-function polypeptide. In some embodiments, the gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the CALCR gene where the gene burden is the number of alleles multiplied by the association estimate with developing obesity and/or increased BMI or related outcome for each allele (e.g., a weighted burden score). This can include any genetic variants, regardless of their genomic annotation, in proximity to the CALCR gene (up to 10 Mb around the gene) that show a non-zero association with obesity- and/or increased BMI-related traits in a genetic association analysis. In some embodiments, when the subject has a gene burden above a desired threshold score, the subject has an increased risk of developing obesity and/or increased BMI. In some embodiments, when the subject has a gene burden below a desired threshold score, the subject has a decreased risk of developing obesity and/or increased BMI.

In some embodiments, the gene burden may be divided into quintiles, e.g., top quintile, intermediate quintile, and bottom quintile, wherein the top quintile of gene burden corresponds to the highest risk group and the bottom quintile of gene burden corresponds to the lowest risk group. In some embodiments, a subject having a greater gene burden comprises the highest weighted gene burdens, including, but not limited to the top 10%, top 20%, top 30%, top 40%, or top 50% of gene burdens from a subject population. In some embodiments, the genetic variants comprise the genetic variants having association with obesity and/or increased BMI in the top 10%, top 20%, top 30%, top 40%, or top 50% of p-value range for the association. In some embodiments, each of the identified genetic variants comprise the genetic variants having association with obesity and/or increased BMI with p-value of no more than about 10⁻², about 10⁻³, about 10⁻⁴, about 10⁻⁵, about 10⁻⁶, about 10⁻⁷, about 10⁻⁸, about 10⁻⁹, about 10-10, about 10-11, about 10-12, about 10-13, about 10-14, about or 10-15. In some embodiments, the identified genetic variants comprise the genetic variants having association with obesity and/or increased BMI with p-value of less than 5×10⁻⁸. In some embodiments, the identified genetic variants comprise genetic variants having association with obesity and/or increased BMI in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having gene burdens in the top decile, quintile, or tertile in a reference population. The threshold of the gene burden is determined on the basis of the nature of the intended practical application and the risk difference that would be considered meaningful for that practical application.

In some embodiments, when a subject is identified as having an increased risk of developing obesity and/or increased BMI, or as having a decreased risk of developing obesity and/or increased BMI, the subject can be treated as described herein.

The present disclosure also provides methods of diagnosing obesity and/or increased BMI in a subject. The methods comprise determining or having determined whether the subject has any one or more of the CALCR variant nucleic acid molecules encoding a CALCR predicted loss-of-function polypeptide described herein. When the subject has a CALCR variant nucleic acid molecule encoding a CALCR predicted loss-of-function polypeptide, and has one or more symptoms of obesity and/or increased BMI, the subject is diagnosed as having obesity and/or increased BMI. In some embodiments, the subject is homozygous for a CALCR variant nucleic acid molecule encoding a CALCR predicted loss-of-function polypeptide. In some embodiments, the subject is heterozygous for a CALCR variant nucleic acid molecule encoding a CALCR predicted loss-of-function polypeptide. In some embodiments, when a subject is identified as having obesity and/or increased BMI (such as having one or more symptoms of obesity and/or increased BMI and being heterozygous or homozygous for a CALCR variant nucleic acid molecule encoding a CALCR predicted loss-of-function polypeptide), the subject is further treated with a therapeutic agent that treats or inhibits obesity and/or increased BMI, such as any of those described herein.

The present disclosure also provides methods of detecting the presence or absence of a CALCR variant genomic nucleic acid molecule encoding a CALCR predicted loss-of-function polypeptide in a biological sample from a subject, and/or a CALCR variant mRNA molecule encoding a CALCR predicted loss-of-function polypeptide in a biological sample from a subject, and/or a CALCR variant cDNA molecule encoding a CALCR predicted loss-of-function polypeptide produced from an mRNA molecule in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any CALCR variant nucleic acid molecule encoding a CALCR predicted loss-of-function polypeptide, preliminary processing designed to isolate or enrich the sample for the genomic DNA can be employed. A variety of techniques may be used for this purpose. When detecting the level of any CALCR variant mRNA molecule, different techniques can be used enrich the biological sample with mRNA. Various methods to detect the presence or level of an mRNA or the presence of a particular variant genomic DNA locus can be used.

In some embodiments, detecting a CALCR variant nucleic acid molecule in a subject comprises assaying or performing a sequence analysis on a biological sample obtained from the subject to determine whether a CALCR genomic nucleic acid molecule in the biological sample, and/or a CALCR mRNA molecule in the biological sample, and/or a CALCR cDNA molecule produced from an mRNA moleucle in the biological sample, comprises one or more variations that cause a loss-of-function (partial or complete) or are predicted to cause a loss-of-function (partial or complete), such as any of the CALCR variant nucleic acid molecules described herein.

In some embodiments, the methods of detecting the presence or absence of a CALCR variant nucleic acid molecule (such as, for example, a genomic nucleic acid molecule, an mRNA molecule, and/or a cDNA molecule produced from an mRNA molecule) encoding a CALCR predicted loss-of-function polypeptide in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a CALCR genomic nucleic acid molecule or mRNA molecule, and if mRNA, optionally reverse transcribing the mRNA into cDNA. Such assays can comprise, for example determining the identity of these positions of the particular CALCR nucleic acid molecule. In some embodiments, the method is an in vitro method.

In some embodiments, the determining step, detecting step, or sequence analysis comprises sequencing at least a portion of the nucleotide sequence of the CALCR genomic nucleic acid molecule, the CALCR mRNA molecule, or the CALCR cDNA molecule in the biological sample, wherein the sequenced portion comprises one or more variations that cause a loss-of-function (partial or complete) or are predicted to cause a loss-of-function (partial or complete), such as any of the CALCR variant nucleic acid molecules described herein.

In some embodiments, the determining step, detecting step, or sequence analysis comprises sequencing at least a portion of the nucleotide sequence of the CALCR genomic nucleic acid molecule in the biological sample, the nucleotide sequence of the CALCR mRNA molecule in the biological sample, or the nucleotide sequence of the CALCR cDNA molecule produced from the CALCR mRNA in the biological sample. In some embodiments, the determining step, detecting step, or genotyping assay comprises sequencing at least a portion of the nucleotide sequence of the CALCR genomic nucleic acid molecule in the biological sample. In some embodiments, the determining step, detecting step, or sequence analysis comprises sequencing at least a portion of the nucleotide sequence of the CALCR mRNA molecule in the biological sample. In some embodiments, the determining step, detecting step, or sequence analysis comprises sequencing at least a portion of the nucleotide sequence of the CALCR cDNA molecule produced from the CALCR mRNA molecule in the biological sample.

In some embodiments, the assay or sequence analysis comprises sequencing the entire nucleic acid molecule. In some embodiments, only a CALCR genomic nucleic acid molecule is analyzed. In some embodiments, only a CALCR mRNA is analyzed. In some embodiments, only a CALCR cDNA obtained from CALCR mRNA is analyzed.

In some embodiments, the determining step, detecting step, or sequence analysis comprises: a) amplifying at least a portion of the nucleic acid molecule that encodes the CALCR polypeptide; b) labeling the amplified nucleic acid molecule with a detectable label; c) contacting the labeled nucleic acid molecule with a support comprising an alteration-specific probe; and d) detecting the detectable label.

In some embodiments, the nucleic acid molecule is mRNA and the determining step further comprises reverse-transcribing the mRNA into a cDNA prior to the amplifying step. In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule; and detecting the detectable label. Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay or sequence analysis comprises contacting the biological sample with a primer or probe, such as an alteration-specific primer or alteration-specific probe, that specifically hybridizes to a CALCR variant genomic nucleic acid molecule, a CALCR variant mRNA molecule, or a CALCR variant cDNA molecule and not the corresponding CALCR reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a CALCR variant genomic nucleic acid molecule, a CALCR variant mRNA molecule, or a CALCR variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a CALCR predicted loss-of-function polypeptide comprising performing an assay on a sample obtained from a subject to determine whether a CALCR polypeptide in the subject contains one or more variations that causes the polypeptide to have a loss-of-function (partial or complete) or predicted loss-of-function (partial or complete), such as any of the CALCR predicted loss-of-function polypeptides described herein. The CALCR predicted loss-of-function polypeptides can be any of the truncated variant CALCR polypeptides described herein.

In some embodiments, the detecting step comprises sequencing at least a portion of the polypeptide. In some embodiments, the detecting step comprises an immunoassay for detecting the presence of a polypeptide.

In some embodiments, when the subject does not have a CALCR predicted loss-of-function polypeptide, the subject does not have an increased risk of developing obesity, such as type 1 obesity, type 2 obesity, or type 3 obesity, or developing increased BMI. In some embodiments, when the subject has a CALCR predicted loss-of-function polypeptide, then the subject has an increased risk of developing obesity, such as type 1 obesity, type 2 obesity, or type 3 obesity, or developing increased BMI.

In the context of the present disclosure “specifically hybridizes” means that the probe or primer (such as, for example, the alteration-specific probe or alteration-specific primer) does not hybridize to a nucleic acid sequence encoding a CALCR reference genomic nucleic acid molecule, a CALCR reference mRNA molecule, and/or a CALCR reference cDNA molecule.

An “alteration-specific probe” specifically hybridizes to a CALCR variant genomic nucleic acid molecule, a CALCR variant mRNA molecule, or a CALCR variant cDNA molecule, and not the corresponding CALCR reference sequence under stringent conditions.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of a CALCR reference genomic nucleic acid molecule is set forth in SEQ ID NO:1 (ENST00000426151.6 encompassing chr7:93,424,539-93,574,730 in the GRCh38/hg38 human genome assembly; ENSG00000004948.15).

The nucleotide sequence of a CALCR reference mRNA molecule is set forth in SEQ ID NO:2. The nucleotide sequence of another CALCR reference mRNA molecule is set forth in SEQ ID NO:3. The nucleotide sequence of another CALCR reference mRNA molecule is set forth in SEQ ID NO:4. The nucleotide sequence of another CALCR reference mRNA molecule is set forth in SEQ ID NO:5. The nucleotide sequence of another CALCR reference mRNA molecule is set forth in SEQ ID NO:6. The nucleotide sequence of another CALCR reference mRNA molecule is set forth in SEQ ID NO:7. The nucleotide sequence of another CALCR reference mRNA molecule is set forth in SEQ ID NO:8. The nucleotide sequence of another CALCR reference mRNA molecule is set forth in SEQ ID NO:9. The nucleotide sequence of another CALCR reference mRNA molecule is set forth in SEQ ID NO:10. The nucleotide sequence of another CALCR reference mRNA molecule is set forth in SEQ ID NO:11. The nucleotide sequence of another CALCR reference mRNA molecule is set forth in SEQ ID NO:12. The nucleotide sequence of another CALCR reference mRNA molecule is set forth in SEQ ID NO:13.

The nucleotide sequence of a CALCR reference cDNA molecule is set forth in SEQ ID NO:14. The nucleotide sequence of another CALCR reference cDNA molecule is set forth in SEQ ID NO:15. The nucleotide sequence of another CALCR reference cDNA molecule is set forth in SEQ ID NO:16. The nucleotide sequence of another CALCR reference cDNA molecule is set forth in SEQ ID NO:17. The nucleotide sequence of another CALCR reference cDNA molecule is set forth in SEQ ID NO:18. The nucleotide sequence of another CALCR reference cDNA molecule is set forth in SEQ ID NO:19. The nucleotide sequence of another CALCR reference cDNA molecule is set forth in SEQ ID NO:20. The nucleotide sequence of another CALCR reference cDNA molecule is set forth in SEQ ID NO:21. The nucleotide sequence of another CALCR reference cDNA molecule is set forth in SEQ ID NO:22. The nucleotide sequence of another CALCR reference cDNA molecule is set forth in SEQ ID NO:23. The nucleotide sequence of another CALCR reference cDNA molecule is set forth in SEQ ID NO:24. The nucleotide sequence of another CALCR reference cDNA molecule is set forth in SEQ ID NO:25.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

The amino acid sequence of a CALCR reference polypeptide is set forth in SEQ ID NO:26. The amino acid sequence of another CALCR reference polypeptide is set forth in SEQ ID NO:27. The amino acid sequence of another CALCR reference polypeptide is set forth in SEQ ID NO:28. The amino acid sequence of another CALCR reference polypeptide is set forth in SEQ ID NO:29. The amino acid sequence of another CALCR reference polypeptide is set forth in SEQ ID NO:30.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit obesity and/or increased BMI for use in the treatment of obesity and/or increased BMI in a subject having: i) a CALCR variant genomic nucleic acid molecule encoding a CALCR predicted loss-of-function polypeptide; ii) a CALCR variant mRNA molecule encoding a CALCR predicted loss-of-function polypeptide; or iii) a CALCR variant cDNA molecule encoding a CALCR predicted loss-of-function polypeptide.

In some embodiments, the subject has any of the CALCR variant genomic nucleic acid molecules encoding a CALCR predicted loss-of-function polypeptide, CALCR variant mRNA molecules encoding a CALCR predicted loss-of-function polypeptide, and/or CALCR variant cDNA molecules encoding a CALCR predicted loss-of-function polypeptide described herein. In some embodiments, the subject has any of the CALCR variant genomic nucleic acid molecules described herein. In some embodiments, the subject has any of the CALCR variant mRNA molecules described herein. In some embodiments, the subject has any of the CALCR variant cDNA molecules described herein. The therapeutic agents that treat or inhibit obesity and/or increased BMI can be any of the therapeutic agents that treat or inhibit obesity and/or increased BMI described herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: CALCR pLOF Variants are Associated with Elevated BMI and Increased Risk of Obesity

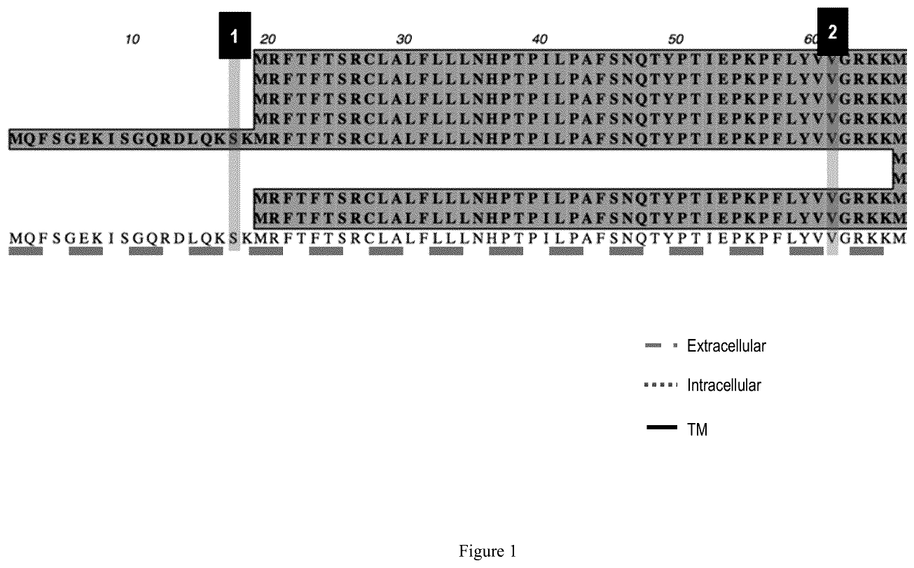

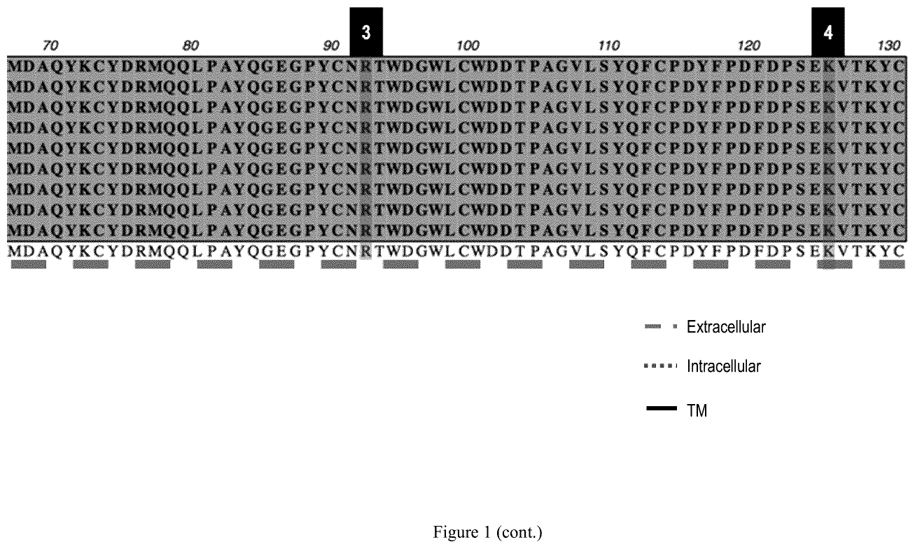

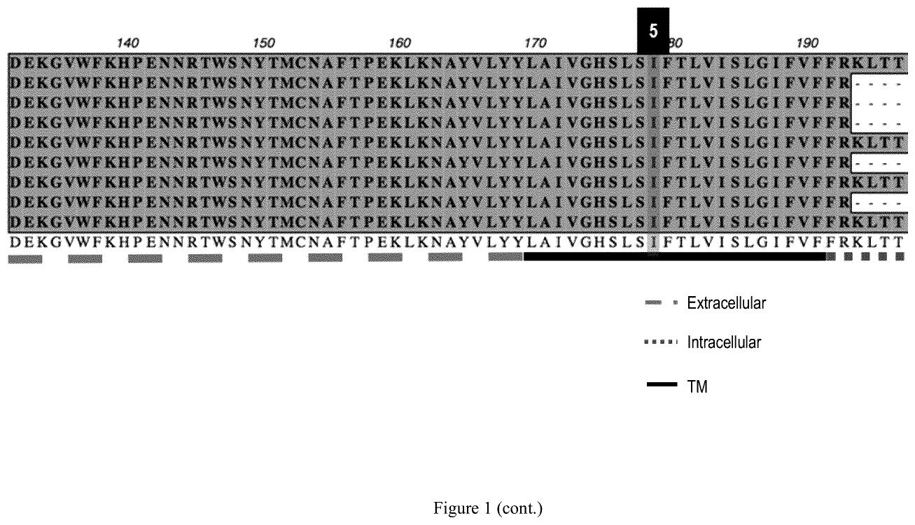

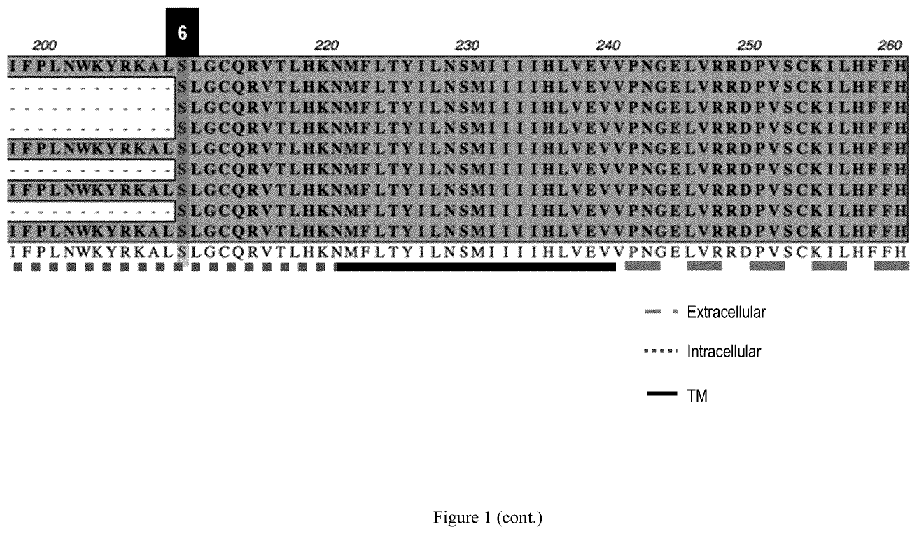

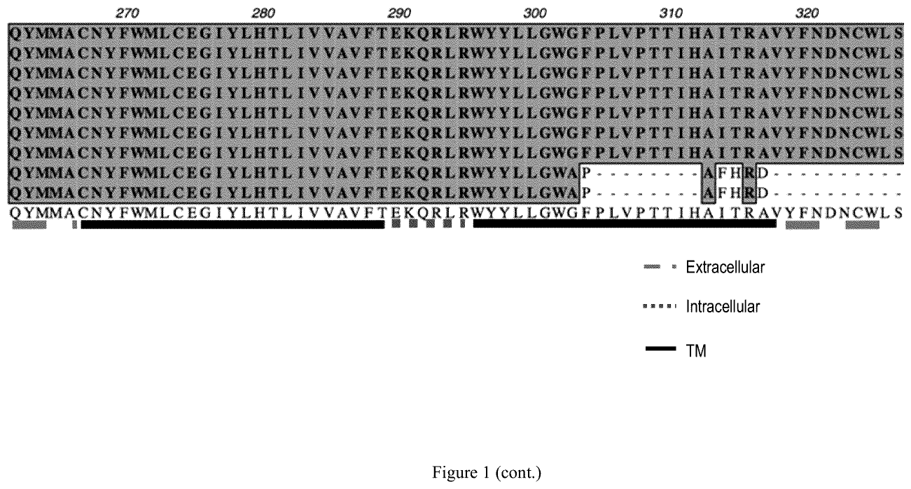

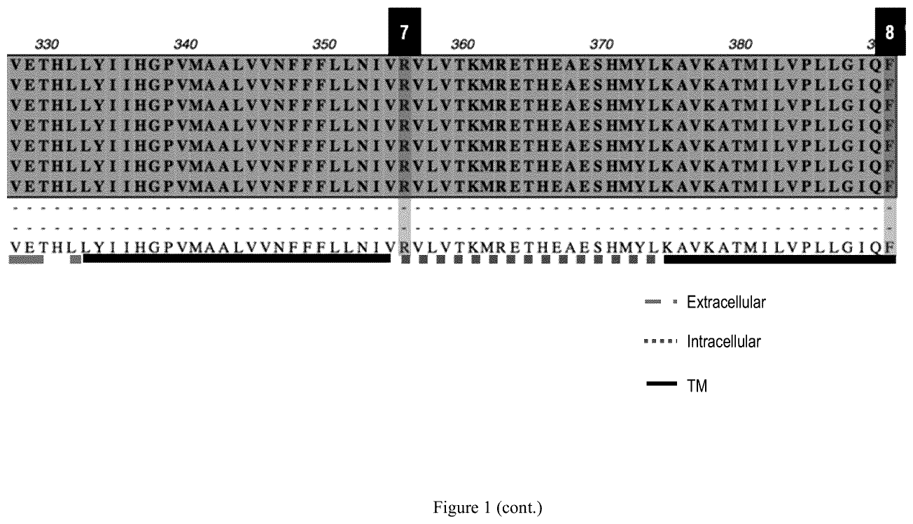

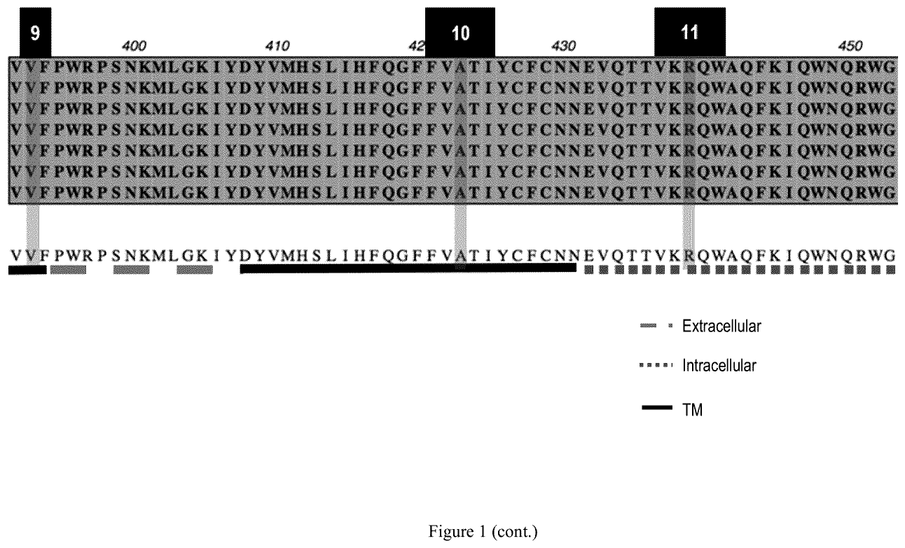

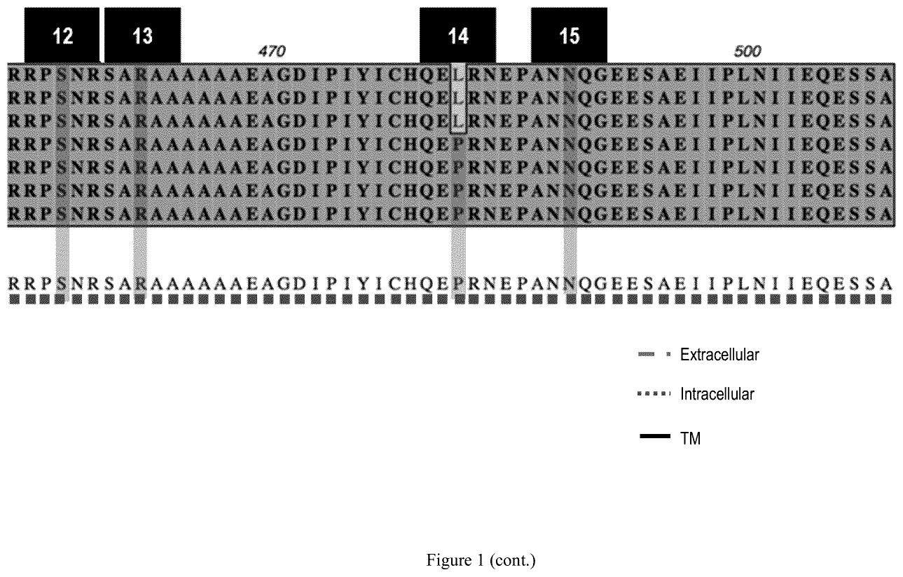

The burden of rare nonsynonymous variants in CALCR and several individual variants in the gene are associated with BMI in humans. A genome-wide association (GWAS) analysis resulted in the association of pLOF missense variants in CALCR with increased BMI and increased obesity risk (see, Table 1 and Table 2), constituting the first human genetic evidence linking LOF in CALCR with obesity. Table 1 summarizes the association between CALCR pLOF variants (or pLOF variants plus missense variants with different functional annotation) and increased BMI. Gene burden tests including pLOF, pLOF plus predicted deleterious missense variants (M3.01, M4.01) or pLOF plus all missense variants were associated with higher BMI (M2.01; see Table 1). Table 2 summarizes the association between CALCR pLOF variants (or pLOF variants plus missense variants with different functional annotation) and higher odds of obesity. Gene burden tests including pLOF, PLOF plus predicted deleterious missense variants (M3.01, M4.01) or pLOF plus all missense variants were associated with higher odds of obesity (M2.01; see Table 2). Table 3 lists CALCR pLOF and missense variants included in the analysis. Of over 100 nonsynonymous variants with individual associations results from the meta-analysis, 15 were associated with BMI at p<0.05, including two variants associated with lower BMI (FIG. 1). Eight of these 15 variants were in the last extracellular, transmembrane and intracellular domains suggesting a possible role for beta-arrestin related pathways in the BMI associations (FIG. 1). In addition, an exom-wide association with BMI was observed (genetic exposure=pLOF plus missense; Beta 95% CI per allele in SD units=0.10 (0.07, 0.13); p-value=1.4×10⁻¹⁰; AAF=0.0049; Beta 95% CI per allele in kg/m2 units=0.6 (0.4, 0.7); and Beta 95% CI per allele in kg of body weight=1.6 (1.1, 2.1).

TABLE 1

| | | Per allele | | | Phenotypic |
|---|---|---|---|---|---|
| | | beta (95% | | | means, |
| | | CI) in | | | kg/m² of |
| Exposure | AAF | SDs | P | Allele counts | BMI |
| pLOF | 0.001 | 0.14 | 4.8E−5 | 406,744: | 31.1\|32.7 |
| | | (0.07, 0.20) | | 405,915\|829\|0 | |
| pLOF or | 0.006 | 0.07 | 5.6E−8 | 406,744: | 31.1\|32.1 |
| missense | | (0.05, 0.10) | | 401,754\|4,989\|1 | |
| (M2.01) | | | | | |
| pLOF or | 0.001 | 0.14 | 4.8E−5 | 406,744: | 31.1\|32.7 |
| missense | | (0.07, 0.20) | | 405,915\|829\|0 | |
| (M3.01) | | | | | |
| pLOF or | 0.005 | 0.10 | 3.0E−10 | 406,744: | 31.1\|32.0 |
| missense | | (0.07, 0.13) | | 402,772\|3,972\|0 | |
| (M4.01) | | | | | |

Abbreviations;
AAF, alternative allele frequency;
CI, confidence interval;
SD, standard deviations;
BMI, body mass index;
pLOF, predicted loss of function.

TABLE 2

| | | Per allele | | | Allele counts- |
|---|---|---|---|---|---|
| Expo- | | Odds ratio | | Allele counts- | non-obese |
| sure | AAF | (95% CI) | P | obese cases | controls |
| pLOF | 0.001 | 1.26 | 0.017 | 132,399: | 120,813: |
| | | (1.04, 1.53) | | 132,083\|316\|0 | 120,580\|233\|0 |
| pLOF | 0.006 | 1.14 | 0.0011 | 132,399: | 120,813: |
| or mis- | | (1.05, 1.24) | | 130,703\|1,696\|0 | 119,398\|1,415\|0 |
| sense | | | | | |
| (M2.01) | | | | | |
| pLOF | 0.001 | 1.26 | 0.017 | 132,399: | 120,813: |
| or mis- | | (1.04, 1.53) | | 132,083\|316\|0 | 120,580\|233\|0 |
| sense | | | | | |
| (M3.01) | | | | | |
| pLOF | 0.005 | 1.21 | 2.4E−05 | 132,399: | 120,813: |
| or mis- | | (1.11, 1.33) | | 130,984\|1,415\|0 | 119,714\|1,099\|0 |
| sense | | | | | |
| (M4.01) | | | | | |

Abbreviations;
AAF, alternative allele frequency;
CI, confidence interval;
pLOF, predicted loss of function.

TABLE 3

| VARIANT | AAF | M1 | M2 | M3 | M4 | Annotation | IS_LOF |
|---|---|---|---|---|---|---|---|
| 7:93426360:G:T | 0.0000065 | false | true | false | true | missense | false |
| 7:93426376:C:T | 0.0000085 | false | true | false | true | missense | false |
| 7:93426378:A:G | 0.000007 | false | true | false | true | missense | false |
| 7:93426384:T:G | 0.0000315 | false | true | false | true | missense | false |
| 7:93426391:G:T | 0.0000065 | false | true | false | true | missense | false |
| 7:93426393:A:T | 0.000013 | false | true | false | true | missense | false |
| 7:93426398:C:G | 0.000049 | false | true | false | true | missense | false |
| 7:93426399:T:C | 0.0000065 | false | true | false | true | missense | false |
| 7:93426404:A:C | 0.0000389 | false | true | false | false | missense | false |
| 7:93426405:C:T | 0.0000175 | false | true | false | false | missense | false |
| 7:93426412:C:A | 0.0000141 | true | true | true | true | stop_gained | true |
| 7:93426412:C:G | 0.0000035 | false | true | false | true | missense | false |
| 7:93426414:C:T | 0.0000035 | false | true | false | true | missense | false |
| 7:93426419:G:C | 0.0000035 | false | true | false | false | missense | false |
| 7:93426419:G:T | 0.000007 | false | true | false | false | missense | false |
| 7:93426422:GT:G | 0.000515 | true | true | true | true | frameshift | true |
| 7:93426433:C:G | 0.0000065 | false | true | false | true | missense | false |
| 7:93426434:AT:A | 0.000042 | true | true | true | true | frameshift | true |
| 7:93426438:C:T | 0.0000035 | false | true | false | false | missense | false |
| 7:93426451:G:A | 0.0000105 | false | true | false | true | missense | false |
| 7:93426459:T:A | 0.0000065 | false | true | false | true | missense | false |
| 7:93426465:G:C | 0.0000065 | false | true | false | true | missense | false |
| 7:93426472:C:T | 0.000007 | false | true | false | false | missense | false |
| 7:93426475:C:T | 0.0000085 | false | true | false | false | missense | false |
| 7:93426477:G:C | 0.0000065 | false | true | false | false | missense | false |
| 7:93426483:G:A | 0.0000065 | false | true | false | true | missense | false |
| 7:93426490:C:T | 0.000007 | false | true | false | true | missense | false |
| 7:93426492:G:T | 0.0000065 | false | true | false | true | missense | false |
| 7:93426496:C:A | 0.0000035 | false | true | false | true | missense | false |
| 7:93426498:G:A | 0.0000035 | false | true | false | false | missense | false |
| 7:93426499:C:T | 0.0000035 | false | true | false | false | missense | false |
| 7:93426501:C:G | 0.0000085 | false | true | false | false | missense | false |
| 7:93426501:CG:C | 0.000014 | true | true | true | true | frameshift | true |
| 7:93426501:C:T | 0.000007 | false | true | false | true | missense | false |
| 7:93426502:G:A | 0.000007 | false | true | false | true | missense | false |
| 7:93426502:G:T | 0.0000085 | false | true | false | false | missense | false |
| 7:93426505:C:G | 0.0000035 | false | true | false | true | missense | false |
| 7:93426511:G:A | 0.0000035 | false | true | false | true | missense | false |
| 7:93426511:G:C | 0.000056 | false | true | false | true | missense | false |
| 7:93426512:G:GT | 0.0000035 | true | true | true | true | frameshift | true |
| 7:93426513:T:C | 0.0000065 | false | true | false | false | missense | false |
| 7:93426514:T:G | 0.0000105 | false | true | false | false | missense | false |
| 7:93426516:G:A | 0.0000324 | false | true | false | false | missense | false |
| 7:93426517:A:AG | 0.000013 | true | true | true | true | frameshift | true |
| 7:93426519:G:T | 0.0000065 | false | true | false | true | missense | false |
| 7:93426520:G:A | 0.0000105 | false | true | false | false | missense | false |
| 7:93426520:G:C | 0.0000085 | false | true | false | false | missense | false |
| 7:93426528:C:T | 0.0000035 | false | true | false | true | missense | false |
| 7:93426529:C:T | 0.0000065 | false | true | false | false | missense | false |
| 7:93426531:C:G | 0.0000035 | false | true | false | true | missense | false |
| 7:93426534:C:T | 0.000028 | false | true | false | true | missense | false |
| 7:93426541:T:C | 0.0000259 | false | true | false | false | missense | false |
| 7:93426545:C:G | 0.0000035 | false | true | false | true | missense | false |
| 7:93426546:T:C | 0.0000035 | false | true | false | true | missense | false |
| 7:93426559:G:A | 0.0000065 | true | true | true | true | stop_gained | true |
| 7:93426561:G:GC | 0.000007 | true | true | true | true | frameshift | true |
| 7:93426562:C:T | 0.000007 | false | true | false | false | missense | false |
| 7:93426563:C:G | 0.0000946 | false | true | false | true | missense | false |
| 7:93426564:C:T | 0.000014 | true | true | true | true | stop_gained | true |
| 7:93426565:A:G | 0.0000035 | false | true | false | true | missense | false |
| 7:93426570:C:T | 0.0000259 | false | true | false | true | missense | false |
| 7:93426571:G:A | 0.0000175 | false | true | false | true | missense | false |
| 7:93426572:C:G | 0.0000035 | false | true | false | true | missense | false |
| 7:93426585:T:C | 0.0000035 | false | true | false | true | missense | false |
| 7:93426588:A:C | 0.0000085 | false | true | false | true | missense | false |
| 7:93434252:C:T | 0.0000065 | true | true | true | true | splice_donor | true |
| 7:93434269:C:G | 0.0000035 | false | true | false | true | missense | false |
| 7:93434281:G:A | 0.0000385 | false | true | false | true | missense | false |
| 7:93434285:C:T | 0.0000105 | false | true | false | true | missense | false |
| 7:93434288:AG:A | 0.0000085 | true | true | true | true | frameshift | true |
| 7:93434289:G:GA | 0.0000085 | true | true | true | true | frameshift | true |
| 7:93435953:T:A | 0.0000065 | false | true | false | true | missense | false |
| 7:93435978:C:T | 0.000042 | false | true | false | true | missense | false |
| 7:93435980:T:C | 0.000014 | false | true | false | true | missense | false |
| 7:93435987:A:G | 0.0000065 | false | true | false | true | missense | false |
| 7:93435988:T:C | 0.000013 | false | true | false | true | missense | false |
| 7:93435991:C:A | 0.0000085 | false | true | false | true | missense | false |
| 7:93436000:C:G | 0.000007 | false | true | false | false | missense | false |
| 7:93436005:T:C | 0.0000085 | false | true | false | true | missense | false |
| 7:93436006:G:T | 0.0000065 | false | true | false | true | missense | false |
| 7:93436007:T:C | 0.0000631 | false | true | false | true | missense | false |
| 7:93436023:G:C | 0.0000035 | false | true | false | true | missense | false |
| 7:93436029:C:T | 0.0000105 | false | true | false | true | missense | false |
| 7:93436032:C:G | 0.000013 | false | true | false | true | missense | false |
| 7:93436035:A:C | 0.000028 | false | true | false | true | missense | false |
| 7:93436053:G:A | 0.0000085 | false | true | false | true | missense | false |
| 7:93436059:G:A | 0.000014 | false | true | false | true | missense | false |
| 7:93436065:T:C | 0.000007 | false | true | false | true | missense | false |
| 7:93436068:T:A | 0.0000194 | false | true | false | true | missense | false |
| 7:93436073:T:C | 0.0000065 | false | true | false | false | missense | false |
| 7:93436079:G:A | 0.0000085 | false | true | false | true | missense | false |
| 7:93436095:G:A | 0.0000245 | false | true | false | false | missense | false |
| 7:93436095:G:T | 0.000014 | false | true | false | false | missense | false |
| 7:93436103:G:A | 0.0000806 | false | true | false | true | missense | false |
| 7:93436103:G:T | 0.0000035 | false | true | false | true | missense | false |
| 7:93436110:G:A | 0.0000085 | false | true | false | true | missense | false |
| 7:93436113:T:A | 0.0000065 | false | true | false | true | missense | false |
| 7:93436116:C:T | 0.000007 | false | true | false | false | missense | false |
| 7:93436122:T:G | 0.0000065 | false | true | false | false | missense | false |
| 7:93436127:G:A | 0.0000065 | false | true | false | true | missense | false |
| 7:93436139:C:G | 0.0000105 | false | true | false | true | missense | false |
| 7:93436139:C:T | 0.0002908 | false | true | false | true | missense | false |
| 7:93436140:G:A | 0.000021 | false | true | false | true | missense | false |
| 7:93436151:A:G | 0.0000035 | false | true | false | true | missense | false |
| 7:93436166:T:C | 0.0000035 | false | true | false | true | missense | false |
| 7:93436169:A:G | 0.0000035 | false | true | false | true | missense | false |
| 7:93436171:C:A | 0.0000105 | true | true | true | true | splice_acceptor | true |
| 7:93436171:C:T | 0.0000085 | true | true | true | true | splice_acceptor | true |
| 7:93436171:CTGC | 0.0000169 | true | true | true | true | splice_acceptor | true |
| AAATATACGG:C | | | | | | | |
| 7:93438056:TCAC:T | 0.0000259 | true | true | true | true | splice_donor | true |
| 7:93438059:C:G | 0.0000035 | true | true | true | true | splice_donor | true |
| 7:93438070:G:A | 0.000021 | false | true | false | true | missense | false |
| 7:93438070:G:T | 0.000028 | false | true | false | true | missense | false |
| 7:93438072:C:A | 0.0000065 | false | true | false | true | missense | false |
| 7:93438073:A:G | 0.0000035 | false | true | false | true | missense | false |
| 7:93438077:C:G | 0.0000035 | false | true | false | true | missense | false |
| 7:93438085:T:C | 0.0000065 | false | true | false | true | missense | false |
| 7:93438089:T:C | 0.0000085 | false | true | false | true | missense | false |
| 7:93438092:T:C | 0.0000254 | false | true | false | true | missense | false |
| 7:93438095:A:C | 0.0000065 | false | true | false | true | missense | false |
| 7:93438101:A:T | 0.0000065 | false | true | false | true | missense | false |
| 7:93438103:T:C | 0.0000035 | false | true | false | true | missense | false |
| 7:93438106:G:A | 0.0000035 | false | true | false | true | missense | false |
| 7:93438109:T:A | 0.0000065 | false | true | false | true | missense | false |
| 7:93438112:A:G | 0.0000065 | false | true | false | true | missense | false |
| 7:93438115:C:T | 0.000007 | false | true | false | true | missense | false |
| 7:93438125:A:G | 0.0000035 | false | true | false | true | missense | false |
| 7:93438209:C:T | 0.000007 | true | true | true | true | splice_donor | true |
| 7:93438226:C:T | 0.000035 | false | true | false | true | missense | false |
| 7:93438229:C:T | 0.0000035 | false | true | false | true | missense | false |
| 7:93438235:T:C | 0.0000085 | false | true | false | false | missense | false |
| 7:93438243:T:A | 0.0000035 | false | true | false | true | missense | false |
| 7:93438247:T:A | 0.000007 | false | true | false | true | missense | false |
| 7:93438255:G:A | 0.0000389 | false | true | false | true | missense | false |
| 7:93438259:C:T | 0.0000453 | false | true | false | true | missense | false |
| 7:93438264:G:A | 0.0000035 | false | true | false | true | missense | false |
| 7:93438270:C:A | 0.0000315 | false | true | false | true | missense | false |
| 7:93443603:C:A | 0.000014 | true | true | true | true | splice_donor | true |
| 7:93443605:C:G | 0.0000035 | false | true | false | true | missense | false |
| 7:93443607:AGC | 0.0000105 | true | true | true | true | frameshift | true |
| CCAAGAGATAATACC:A | | | | | | | |
| 7:93443621:T:C | 0.000007 | false | true | false | true | missense | false |
| 7:93443622:A:G | 0.000013 | false | true | false | true | missense | false |
| 7:93443624:C:A | 0.0000736 | false | true | false | true | missense | false |
| 7:93443624:C:G | 0.0000065 | false | true | false | true | missense | false |
| 7:93443627:C:T | 0.0000981 | false | true | false | false | missense | false |
| 7:93443628:G:A | 0.0000525 | false | true | false | true | missense | false |
| 7:93443633:C:T | 0.000007 | false | true | false | false | missense | false |
| 7:93443634:G:A | 0.0000876 | false | true | false | true | missense | false |
| 7:93443634:G:T | 0.000007 | false | true | false | false | missense | false |
| 7:93443648:AAC:A | 0.0000035 | true | true | true | true | frameshift | true |
| 7:93443657:A:G | 0.0000065 | false | true | false | true | missense | false |
| 7:93443658:C:T | 0.0000385 | false | true | false | true | missense | false |
| 7:93443666:A:C | 0.0000085 | false | true | false | true | missense | false |
| 7:93443681:A:T | 0.0000035 | false | true | false | true | missense | false |
| 7:93443682:T:A | 0.000013 | false | true | false | true | missense | false |
| 7:93443682:T:G | 0.0000105 | false | true | false | true | missense | false |
| 7:93443696:A:G | 0.000007 | false | true | false | true | missense | false |
| 7:93443703:A:G | 0.0000035 | false | true | false | true | missense | false |
| 7:93443705:T:C | 0.0000035 | false | true | false | true | missense | false |
| 7:93443711:C:G | 0.0000035 | false | true | false | true | missense | false |
| 7:93443714:G:C | 0.0000035 | false | true | false | false | missense | false |
| 7:93443718:T:C | 0.0000035 | false | true | false | true | missense | false |
| 7:93443719:C:G | 0.0000035 | false | true | false | true | missense | false |
| 7:93443720:A:G | 0.000013 | false | true | false | true | missense | false |
| 7:93443727:G:C | 0.0000035 | false | true | false | true | missense | false |
| 7:93443727:G:T | 0.000014 | false | true | false | true | missense | false |
| 7:93443729:T:A | 0.0000035 | false | true | false | true | missense | false |
| 7:93443733:A:G | 0.0000035 | false | true | false | true | missense | false |
| 7:93443734:A:T | 0.0000065 | false | true | false | true | missense | false |
| 7:93443739:G:C | 0.0000035 | false | true | false | true | missense | false |
| 7:93443743:AATCTT:A | 0.0000035 | true | true | true | true | frameshift | true |
| 7:93443746:C:G | 0.000007 | false | true | false | true | missense | false |
| 7:93443750:C:T | 0.0000085 | false | true | false | true | missense | false |
| 7:93443758:C:T | 0.0000259 | true | true | true | true | splice_acceptor | true |
| 7:93460822:G:A | 0.0000105 | false | true | false | true | missense | false |
| 7:93460822:G:T | 0.0002592 | false | true | false | true | missense | false |
| 7:93460826:C:A | 0.0000085 | false | true | false | true | missense | false |
| 7:93460828:C:T | 0.0000712 | false | true | false | false | missense | false |
| 7:93460831:C:T | 0.0000065 | false | true | false | false | missense | false |
| 7:93460832:G:A | 0.0000105 | true | true | true | true | stop_gained | true |
| 7:93460835:C:A | 0.0000085 | false | true | false | true | missense | false |
| 7:93460835:C:T | 0.000007 | false | true | false | true | missense | false |
| 7:93460838:G:A | 0.000013 | false | true | false | true | missense | false |
| 7:93460844:C:T | 0.0000085 | false | true | false | false | missense | false |
| 7:93460845:A:C | 0.0000035 | false | true | false | true | missense | false |
| 7:93460846:T:C | 0.0000065 | false | true | false | true | missense | false |
| 7:93460856:C:T | 0.0000035 | false | true | false | true | missense | false |
| 7:93460867:T:C | 0.0000035 | false | true | false | true | missense | false |
| 7:93460881:C:T | 0.0000065 | false | true | false | false | missense | false |
| 7:93460882:A:G | 0.0000035 | false | true | false | false | missense | false |
| 7:93460897:T:C | 0.0000169 | false | true | false | true | missense | false |
| 7:93460906:A:G | 0.0000065 | false | true | false | true | missense | false |
| 7:93460918:T:C | 0.0000065 | false | true | false | true | missense | false |
| 7:93460924:G:T | 0.0000035 | false | true | false | true | missense | false |
| 7:93460929:C:A | 0.0000065 | false | true | false | true | missense | false |
| 7:93460930:C:T | 0.0000065 | false | true | false | true | missense | false |
| 7:93460931:T:C | 0.0000105 | false | true | false | true | missense | false |
| 7:93460939:C:T | 0.0000065 | false | true | false | true | missense | false |
| 7:93460945:C:T | 0.0002627 | false | true | false | true | missense | false |
| 7:93460949:T:C | 0.0000105 | true | true | true | true | splice_acceptor | true |
| 7:93462060:T:TA | 0.0000876 | true | true | true | true | splice_donor | true |
| 7:93462067:C:A | 0.0000035 | false | true | false | true | missense | false |
| 7:93462090:G:A | 0.000007 | false | true | false | true | missense | false |
| 7:93462091:G:A | 0.0000035 | false | true | false | true | missense | false |
| 7:93462104:C:G | 0.000007 | false | true | false | true | missense | false |
| 7:93462112:T:C | 0.0000035 | true | true | true | true | splice_acceptor | true |
| 7:93468717:G:C | 0.0000035 | false | true | false | true | missense | false |
| 7:93468722:A:C | 0.000007 | false | true | false | true | missense | false |
| 7:93468725:C:T | 0.0000065 | false | true | false | false | missense | false |
| 7:93468750:G:A | 0.0000065 | false | true | false | false | missense | false |
| 7:93468750:G:C | 0.0000035 | false | true | false | false | missense | false |
| 7:93468758:T:C | 0.0009528 | false | true | false | true | missense | false |
| 7:93468759:T:C | 0.0000065 | false | true | false | true | missense | false |
| 7:93468769:T:G | 0.0000324 | false | true | false | true | missense | false |
| 7:93468781:G:A | 0.0000065 | false | true | false | true | missense | false |
| 7:93468781:G:T | 0.0000035 | false | true | false | true | missense | false |
| 7:93468782:C:T | 0.0000085 | false | true | false | true | missense | false |
| 7:93468787:T:G | 0.0000035 | false | true | false | true | missense | false |
| 7:93468789:G:T | 0.0000035 | true | true | true | true | stop_gained | true |
| 7:93468797:C:T | 0.000007 | false | true | false | false | missense | false |
| 7:93468799:T:C | 0.0001366 | false | true | false | true | missense | false |
| 7:93472373:A:G | 0.0000065 | true | true | true | true | splice_donor | true |
| 7:93472379:A:G | 0.0000035 | false | true | false | true | missense | false |
| 7:93472383:T:C | 0.000007 | false | true | false | true | missense | false |
| 7:93472384:C:CT | 0.0000841 | true | true | true | true | frameshift | true |
| 7:93472392:T:G | 0.0000035 | false | true | false | true | missense | false |
| 7:93472397:G:C | 0.0000453 | false | true | false | true | missense | false |
| 7:93472398:C:A | 0.0000065 | false | true | false | false | missense | false |
| 7:93472412:T:C | 0.000007 | false | true | false | true | missense | false |
| 7:93472415:T:C | 0.000013 | false | true | false | true | missense | false |
| 7:93472418:G:C | 0.0000035 | false | true | false | true | missense | false |
| 7:93472418:G:T | 0.0002802 | false | true | false | true | missense | false |
| 7:93472420:C:T | 0.0000065 | true | true | true | true | stop_gained | true |
| 7:93472424:G:A | 0.0000085 | false | true | false | true | missense | false |
| 7:93472427:C:T | 0.000056 | false | true | false | true | missense | false |
| 7:93472428:G:A | 0.0000035 | true | true | true | true | stop_gained | true |
| 7:93472446:T:TA | 0.0000175 | true | true | true | true | frameshift | true |
| 7:93472454:A:G | 0.0000175 | false | true | false | false | missense | false |
| 7:93472458:C:A | 0.000013 | false | true | false | true | missense | false |
| 7:93472465:A:T | 0.000007 | false | true | false | false | missense | false |
| 7:93472472:T:C | 0.0000065 | false | true | false | true | missense | false |
| 7:93472473:A:G | 0.000013 | false | true | false | true | missense | false |
| 7:93472479:T:C | 0.0000035 | false | true | false | false | missense | false |
| 7:93472482:C:A | 0.0000065 | false | true | false | true | missense | false |
| 7:93472483:CT:C | 0.0000105 | true | true | true | true | frameshift | true |
| 7:93477556:A:C | 0.0000035 | true | true | true | true | splice_donor | true |
| 7:93477557:C:A | 0.000013 | true | true | true | true | splice_donor | true |
| 7:93477566:T:A | 0.0000259 | false | true | false | true | missense | false |
| 7:93477575:G:A | 0.0000035 | false | true | false | true | missense | false |
| 7:93477584:T:C | 0.0000105 | false | true | false | true | missense | false |
| 7:93477590:C:T | 0.0000065 | false | true | false | true | missense | false |
| 7:93477599:T:C | 0.0000254 | false | true | false | true | missense | false |
| 7:93477599:T:G | 0.0000035 | false | true | false | false | missense | false |
| 7:93477602:G:A | 0.0000035 | false | true | false | true | missense | false |
| 7:93477611:C:A | 0.0000035 | false | true | false | true | missense | false |
| 7:93477612:C:G | 0.0000035 | false | true | false | true | missense | false |
| 7:93477617:G:A | 0.0000596 | false | true | false | true | missense | false |
| 7:93477617:G:C | 0.0000035 | false | true | false | true | missense | false |
| 7:93477617:G:T | 0.0000035 | false | true | false | true | missense | false |
| 7:93477620:G:A | 0.0000035 | false | true | false | true | missense | false |
| 7:93477622:G:C | 0.0000035 | false | true | false | true | missense | false |
| 7:93477622:G:T | 0.0000105 | false | true | false | true | missense | false |
| 7:93477623:T:C | 0.0000035 | false | true | false | true | missense | false |
| 7:93477626:T:G | 0.0000065 | false | true | false | true | missense | false |
| 7:93477628:CCAGCA:C | 0.000007 | true | true | true | true | frameshift | true |
| 7:93477632:C:A | 0.0000065 | false | true | false | true | missense | false |
| 7:93477637:C:G | 0.0000065 | false | true | false | true | missense | false |
| 7:93477638:C:G | 0.0000035 | false | true | false | true | missense | false |
| 7:93477639:A:G | 0.000007 | false | true | false | true | missense | false |
| 7:93477644:T:A | 0.0000194 | false | true | false | true | missense | false |
| 7:93477650:G:T | 0.0000035 | false | true | false | true | missense | false |
| 7:93477653:C:A | 0.0000085 | false | true | false | true | missense | false |
| 7:93477653:C:T | 0.000014 | false | true | false | true | missense | false |
| 7:93477654:G:A | 0.0000525 | false | true | false | true | missense | false |
| 7:93477654:G:C | 0.0000065 | false | true | false | true | missense | false |
| 7:93477654:G:T | 0.000007 | false | true | false | true | missense | false |
| 7:93477656:T:C | 0.000007 | false | true | false | true | missense | false |
| 7:93477660:AAT:A | 0.0000085 | true | true | true | true | frameshift | true |
| 7:93477662:T:C | 0.0000065 | false | true | false | true | missense | false |
| 7:93477668:C:A | 0.0000035 | false | true | false | true | missense | false |
| 7:93479360:C:T | 0.0000339 | false | true | false | false | missense | false |
| 7:93479369:C:T | 0.000021 | false | true | false | false | missense | false |
| 7:93479371:G:A | 0.0000324 | false | true | false | true | missense | false |
| 7:93479372:G:A | 0.000007 | false | true | false | true | missense | false |
| 7:93479386:C:T | 0.000007 | false | true | false | true | missense | false |
| 7:93479387:G:A | 0.000014 | true | true | true | true | stop_gained | true |
| 7:93479404:T:C | 0.0000065 | false | true | false | true | missense | false |
| 7:93479410:T:C | 0.0000525 | false | true | false | true | missense | false |
| 7:93479425:C:T | 0.000028 | false | true | false | true | missense | false |
| 7:93479426:G:A | 0.000035 | true | true | true | true | stop_gained | true |
| 7:93479426:G:C | 0.0000065 | false | true | false | true | missense | false |
| 7:93479428:C:T | 0.000007 | false | true | false | false | missense | false |
| 7:93479432:C:T | 0.0009739 | false | true | false | false | missense | false |
| 7:93479435:C:T | 0.000007 | false | true | false | false | missense | false |
| 7:93479449:T:G | 0.0000035 | false | true | false | false | missense | false |
| 7:93479450:T:TG | 0.0000035 | true | true | true | true | frameshift | true |
| 7:93479453:G:T | 0.0000385 | false | true | false | true | missense | false |
| 7:93479456:C:T | 0.0000259 | false | true | false | true | missense | false |
| 7:93479470:G:T | 0.0000035 | false | true | false | true | missense | false |
| 7:93479472:T:A | 0.0000175 | false | true | false | false | missense | false |
| 7:93479489:G:C | 0.0000385 | false | true | false | false | missense | false |
| 7:93479501:T:C | 0.0000035 | false | true | false | false | missense | false |
| 7:93479501:T:G | 0.0000085 | false | true | false | false | missense | false |
| 7:93486931:A:T | 0.0000035 | false | true | false | false | missense | false |
| 7:93486932:T:G | 0.0000035 | false | true | false | false | missense | false |
| 7:93486939:G:C | 0.0000035 | false | true | false | false | missense | false |
| 7:93486943:A:C | 0.0000907 | false | true | false | false | missense | false |
| 7:93486944:A:G | 0.0000065 | false | true | false | true | missense | false |
| 7:93486945:A:T | 0.0000035 | false | true | false | false | missense | false |
| 7:93486951:C:T | 0.0000085 | false | true | false | false | missense | false |
| 7:93486956:C:T | 0.0000085 | false | true | false | false | missense | false |
| 7:93486957:A:T | 0.0000065 | false | true | false | true | missense | false |
| 7:93486959:C:T | 0.0000175 | false | true | false | false | missense | false |
| 7:93486962:C:A | 0.0000065 | false | true | false | false | missense | false |
| 7:93486962:C:T | 0.0000035 | false | true | false | false | missense | false |
| 7:93486965:G:A | 0.0000035 | false | true | false | false | missense | false |
| 7:93486979:CATTTTTG:C | 0.0000175 | true | true | true | true | frameshift | true |
| 7:93486996:G:T | 0.0000065 | false | true | false | false | missense | false |
| 7:93487001:CTT:C | 0.0000065 | true | true | true | true | frameshift | true |
| 7:93487005:G:T | 0.0000065 | false | true | false | true | missense | false |
| 7:93487008:C:T | 0.000007 | true | true | true | true | splice_acceptor | true |
| 7:93495902:G:T | 0.0000035 | true | true | true | true | splice_donor | true |
| 7:93495903:C:T | 0.000014 | true | true | true | true | splice_donor | true |
| 7:93495930:A:G | 0.0000035 | true | true | true | true | start_lost | true |
| 7:93495976:T:A | 0.0000105 | true | true | true | true | splice_acceptor | true |
| 7:93495976:T:C | 0.000021 | true | true | true | true | splice_acceptor | true |

### Example 2: Expanded CWAS of CALCR Association with Elevated BMI and Increased Risk of Obesity

### Participating Cohorts

Discovery genetic association studies were performed in the United Kingdom (UK) Biobank (UKB) cohort, in the MyCode Community Health Initiative cohort from the Geisinger Health System (GHS), and in the Mexico City Prospective Study (MCPS). The UKB is a population-based cohort study of people aged between 40 and 69 years recruited through 22 testing centers in the UK between 2006-2010. A total of 428,719 European ancestry participants with available whole-exome sequencing and clinical phenotype data were included (Table 4). The GHS MyCode study is a health system-based cohort of patients from Central and Eastern Pennsylvania (USA) recruited in 2007-2019. A total of 121,061 European ancestry participants with available whole-exome sequencing and clinical phenotype data were included (Table 4). The MCPS is a cohort study of people aged >35 years recruited from two contiguous urban districts in Mexico City in 2000-2004. A total of 95,846 individuals of Admixed American ancestry with available whole-exome sequencing and clinical phenotype data were included (Table 4).

TABLE 4
Baseline characteristics of individuals included in the exome-wide
association study

| | UKB study | GHS study | MCPS study |
|---|---|---|---|
| Variable | (N = 428,719) | (N = 121,061) | (N = 95,846) |
| Age, mean (SD) | 57 (8) | 53 (17) | 52 (13) |
| in years | | | |
| Women, N (%) | 232,553 (54) | 73,769 (61) | 65,330 (68) |
| Body mass | 27.4 (4.8) | 31.1 (7.3) | 29.1 (5.1) |
| index, mean (SD) | | | |
| in kg/m² | | | |
| Body weight, | 78 (16) | 88 (23) | 70 (14) |
| mean (SD) in kg | | | |
| Blood pressure, | | | |
| mean (SD) in | | | |
| mmHg | | | |
| Systolic | 138 (19) | 124 (11) | 127 (17) |
| Diastolic | 82 (11) | 74 (7) | 83 (10) |
| Low-density | 138 (34) | 107 (29) | Not |
| lipoprotein | | | measured |
| cholesterol, mean | | | |
| (SD) in mg/dL | | | |
| Triglycerides, | 132 (93, 191) | 124 (90, 172) | Not |
| median (IQR) | | | measured |
| in mg/dL | | | |

Abbreviations:
UKB, UK Biobank;
GHS, Geisinger Health System;
MCPS, Mexico City Prospective Study;
SD, standard deviation;
N, number of participants;
IQR, interquartile range.

### Phenotype Definitions

Body mass index (BMI) was calculated as weight in kilograms divided by the square of height in meters on the basis of anthropometric measurements taken at one of the study visits. BMI categories were defined on the basis of the World Health Organization classification. BMI values were transformed by the inverse standard normal function, applied within each ancestry group and separately in men and women. Overall and regional body lean and fat masses, percentages and body-surface normalized indices were measured by bioelectrical impedance in the UKB cohort.

### Genotype Data

High coverage whole exome sequencing was performed as previously described. NimbleGen probes (VCRome) or a modified version of the xGen design available from Integrated DNA Technologies (IDT) were used for target sequence capture. Sequencing was performed using 75 bp paired-end reads on Illumina v4 HiSeq 2500 or NovaSeq instruments. Sequencing had a coverage depth (ie, number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Sequence read alignment and variant calling was based on the GRCh38 Human Genome reference sequence. Ensembl v85 gene definitions were used to determine the functional impact of single nucleotide variants and insertion-deletions. Predicted LOF genetic variants included: a) insertions or deletions resulting in a frameshift; b) insertions, deletions or single nucleotide variants resulting in the introduction of a premature stop codon or in the loss of the transcription start site or stop site; and c) variants in donor or acceptor splice sites. Missense variants were classified for likely functional impact according to the number of in silico prediction algorithms that predicted deleteriousness using SIFT, Polyphen2_HDIV and Polyphen2_HVAR, LRT and MutationTaster. For each gene, the alternative allele frequency (AAF) and functional annotation of each variant determined inclusion into these 7 gene burden exposures: 1) pLOF variants with AAF <1%; 2) pLOF or missense variants predicted deleterious by 5/5 algorithms with AAF <1%; 3) pLOF or missense variants predicted deleterious by 5/5 algorithms with AAF <0.1%; 4) pLOF or missense variants predicted deleterious by at least 1/5 algorithms with AAF <1%; 5) pLOF or missense variants predicted deleterious by at least 1/5 algorithms with AAF <0.1%; 6) pLOF or any missense with AAF <1%; and 7) pLOF or any missense variants with AAF <0.1%.

### Generation of a Genome Wide-Polygenic Score for BMI

SNP array genotyping was performed in the UKB as previously described. A polygenic score capturing predisposition to higher BMI due to over 2.5 million common variants was generated using the LDpred software from the results of a previous large genome-wide association study in an independent dataset.

### Statistical Analysis

The association with BMI of genetic variants or their gene burden were estimated by fitting mixed-effects regression models using BOLT-LMM v2.3.442 or REGENIE v1.0.43 These approaches account for relatedness and population structure by estimating a polygenic score using genotypes from across the genome. Then, the association of genetic variants or their burden is estimated conditional upon that polygenic score as a covariate. Analyses were further adjusted for age, age2, sex, an age-by-sex interaction term, experimental batch-related covariates, and genetic principal components. To ensure that burden associations were statistically independent of BMI-associated common genetic variants, the gene burden association analyses was further adjusted for common variants identified by fine-mapping genome-wide associations of common alleles with BMI as described in the below. Results across cohorts were pooled using inverse-variance weighted meta-analysis.

The following analyses were performed by the following steps:
1. BMI-associated common variants were identified by performing a genome-wide association study including over 12 million common-to-low-frequency genetic variants imputed using the Haplotype Reference Consortium panel. In the GHS study, imputation was performed separately in samples genotyped with the Illumina Human Omni Express Exome array (OMNI set) and the Global Screening array (GSA set). Dosage data from imputed variants were then merged across the two GHS sets, to obtain a combined dataset for association analysis. Genome-wide association analyses were performed in the GHS, UKB and MCPS cohorts separately by fitting mixed-effects linear regression models using BOLT-LMM or REGENIE. Results from the UKB and GHS analyses were then combined by inverse variance-weighted meta-analysis to obtain a genome-wide meta-analysis in the European subset of the discovery cohorts.
2. To identify conditionally-independent genetic association signals driven by common variants, fine-mapping at genomic regions harboring genetic variants associated with BMI was performed at the genome-wide significance threshold of p<5×10⁻⁰⁸ using the FINEMAP software. Linkage disequilibrium was estimated using genetic data from the exact set of individuals included in the genome-wide association analyses. Fine-mapping was performed separately in the meta-analysis of the European ancestry GHS and UKB cohorts and in the Admixed American ancestry analysis in the MCPS cohort.
3. For each locus that was fine-mapped, the 95% credible variant set was identified that had the highest posterior probability of being causal and retained the lead common variant(s) (minor allele frequency >1%) for that set. In total, 1,769 independent associations with common variants in the European ancestry subset and 134 associations in the smaller Admixed American subset were identified.
4. In each cohort, exome-wide association analyses for gene burden associations was performed using as covariates the genotypes of BMI-associated common variants identified by fine-mapping within the relevant ancestry group for that cohort. The results of the exome-wide analyses within each cohort were then meta-analyzed to obtain estimates for gene burden associations that are independent of BMI-associated common variants (Table 5; CALCR; genomic coordinates 7:93426355; pLOF plus missesnse (1/5); AAF <0.1%).

### Results

In an exome-wide analysis of 645,626 individuals, CALCR was one of sixteen genes for which the burden of rare nonsynonymous genetic variants was associated with BMI at the exome-wide level of statistical significance (p<3.6×10⁻⁰⁷, a Bonferroni correction for 20,000 genes and seven variant selection models; Table 5). The CALCR association was consistent across the constituent datasets of the meta-analysis (Table 6).

Five of the sixteen genes encode G-protein coupled receptors (GPCRs; the largest class of drug targets in the human genome) expressed in the brain and central nervous system, including CALCR. This study provides the first human genetic evidence linking rare coding variation in CALCR to BMI and obesity-related phenotypes.

TABLE 5

| Beta (95% | | | | Beta | Beta | Beta |
|---|---|---|---|---|---|---|
| CI) per | | | Genotype | (95% CI) | (95% CI) | (95% CI) |
| allele in | | AAF, | Counts | per allele | per allele | per allele |
| SD units | | fraction | RR\|RA\|AA | in kg/m² | in kgs of | in lbs of |
| of BMI | P | of 1 | genotypes | units of BMI | body weight | body weight |
| 0.08 | 1.9 × 10⁻¹² | 0.0048 | 639,416\|6,208\|2 | 0.5 | 1.3 | 2.9 |
| (0.06, 0.11) | | | | (0.3, 0.6) | (1.0, 1.7) | (2.1, 3.7) |

Table 5 reports genes for which the gene burden of rare nonsynonymous variants was associated with body mass index at the exome-wide level of statistical significance (p<3.6×10⁻⁷). Analyses were performed in 645,626 participants from the UKB, GHS and MCPS studies. Genomic coordinates reflect chromosome and physical position in base pairs according to Genome Reference Consortium Human Build 38. Abbreviations: CI, confidence interval; SD, standard deviation; BMI, body mass index; AAF, alternative allele frequency; RR, reference-reference genotype; RA, reference-alternative heterozygous genotype; AA, alternative-alternative homozygous genotype; pLOF, predicted loss of function; Missense (1/5), missense variant predicted to be deleterious by at least 1 out of 5 in silico prediction algorithms; Missense (5/5), missense variant predicted to be deleterious by 5 out of 5 in silico prediction algorithms.

TABLE 6
CALCR association results in UKB, GHS and MCPS cohorts
with body mass index in the exome-wide analysis

| Genetic | | Beta | | | |
|---|---|---|---|---|---|
| exposure, | | (95% | | | |
| variant type; | | CI) per | | | Genotype |
| allele fre- | | allele in | | AAF, | counts, |
| quency cut- | | SD units | | fraction | RR\|RA\|AA |
| off in % | Study | of BMI | p | of 1 | genotypes |
| pLOF plus | UKB | 0.07 | 6.7E−06 | 0.0049 | 424,507\|4,212\|0 |
| deleterious | | (0.04, | | | |
| missense | | 0.09) | | | |
| (1/5); | | | | | |
| AAF < 0.1% | | | | | |
| pLOF plus | MCPS | 0.09 | 7.0E−03 | 0.0044 | 94,999\|845\|2 |
| deleterious | | (0.02, | | | |
| missense | | 0.16) | | | |
| (1/5); | | | | | |
| AAF < 0.1% | | | | | |
| pLOF plus | GHS | 0.17 | 6.9E−10 | 0.0048 | 119,910\|1,151\|0 |
| deleterious | | (0.11, | | | |
| missense | | 0.22) | | | |
| (1/5); | | | | | |
| AAF < 0.1% | | | | | |

Abbreviations:
BMI, body mass index;
SD, standard deviations;
pLOF, predicted loss of function;
AAF, alternative allele frequency;
RR, reference-reference genotype;
RA, reference-alternative heterozygous genotype;
AA, alternative-alternative homozygous genotype;
UKB, UK Biobank;
GHS, Geisinger Health System MyCode study;
MCPS, Mexico City Prospective Study

A novel association of the burden of rare (AAF <0.1%) CALCR pLOF mutations was identified and predicted-deleterious missense variants in the calcitonin receptor gene with 0.08 SDs (˜0.5 kg/m²) higher BMI and 20% higher odds of obesity (OR, 1.20; 95% CI, 1.12, 1.20; p=8.9×10⁻⁰⁷; Table 5 and Table 7). In addition, the burden of CALCR pLOF genetic variants alone (such as, excluding missense variants) was associated with higher BMI (Table 8), indicating that loss-of-function in CALCR is associated with higher BMI and obesity risk in humans

TABLE 7
CALCR Association with risk of obesity in a meta-analysis of UKB,
GHS and MCPS for the 16 genes associated with body mass index
in the exome-wide analysis and for the two loss-of-function missense
variants in GIPR

| Genetic | | | | | |
|---|---|---|---|---|---|
| exposure, | | | | | |
| variant | | | | Genotype | Genotype |
| type; | Per-allele | | | counts | counts |
| allele | OR | | AAF, | (cases), | (controls), |
| frequency | (95% CI) | | fraction | RR\|RA\|AA | RR\|RA\|AA |
| cut-off in % | for obesity | p | of 1 | genotypes | genotypes |
| pLOF plus | 1.20 | 8.9E−07 | 0.00484 | 198,592\| | 184,749\| |
| deleterious | (1.12, 1.29) | | | 2,065\|1 | 1,680\|0 |
| missense | | | | | |
| (1/5); AAF < | | | | | |
| 0.1% | | | | | |

Abbreviations:
OR, odds ratio;
CI, confidence intervals;
pLOF, predicted loss of function;
AAF, alternative allele frequency.
Results are from a meta-analysis of the UKB, GHS and MCPS studies.

TABLE 8
CALCR association with BMI in ancillary analyses of this study

| Genetic exposure, | | | |
|---|---|---|---|
| variant type; | | Beta (95% CI) | |
| frequency | | per allele in SD | |
| cutoff in % | Description | units of BMI | p |
| pLOF; AAF < 1% | Main analysis | 0.10 | 2.3E−04 |
| | | (0.05, 0.15) | |

Abbreviations:
CI, confidence intervals;
SD, standard deviations;
BMI, body mass index;
pLOF, predicted loss of function;
AAF, alternative allele frequency.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

What is claimed is:

1. A method of treating a subject with a therapeutic agent that treats or inhibits obesity and/or reduces body mass index (BMI), wherein the subject has obesity and/or increased BMI, the method comprising the steps of:
determining whether the subject has a Calcitonin Receptor (CALCR) variant nucleic acid molecule encoding a CALCR predicted loss-of-function polypeptide by:
obtaining or having obtained a biological sample from the subject; and
performing or having performed a sequence analysis on the biological sample to determine if the subject has a genotype comprising the CALCR variant nucleic acid molecule; and
administering or continuing to administer to the subject the therapeutic agent that treats or inhibits obesity and/or increased BMI in a standard dosage amount to a subject that is CALCR reference; or
administering or continuing to administer to the subject the therapeutic agent that treats or inhibits obesity and/or increased BMI in an amount that is the same as or greater than a standard dosage amount to a subject that is heterozygous or homozygous for the CALCR variant nucleic acid molecule;
wherein the presence of a genotype having the CALCR variant nucleic acid molecule encoding a CALCR predicted loss-of-function polypeptide indicates the subject has an increased risk of developing obesity and/or increased BMI.

2. The method according to claim 1, wherein the CALCR variant nucleic acid molecule is a genomic nucleic acid molecule.

3. The method according to claim 2, wherein the sequence analysis comprises sequencing at least a portion of the nucleotide sequence of the CALCR genomic nucleic acid molecule in the biological sample.

4. The method according to claim 1, wherein the CALCR variant nucleic acid molecule is an mRNA molecule.

5. The method according to claim 4, wherein the sequence analysis comprises sequencing at least a portion of the nucleotide sequence of the CALCR mRNA molecule in the biological sample.

6. The method according to claim 1, wherein the CALCR variant nucleic acid molecule is a cDNA molecule produced from an mRNA molecule.

7. The method according to claim 6, wherein the sequence analysis comprises sequencing at least a portion of the nucleotide sequence of the CALCR cDNA molecule produced from an mRNA molecule in the biological sample.

8. The method according to claim 1, wherein the CALCR variant nucleic acid molecule encoding a CALCR predicted loss-of-function polypeptide is a missense variant, a splice-site variant, a stop-gain variant, a start-loss variant, a stop-loss variant, a frameshift variant, or an in-frame indel variant, or a variant that encodes a truncated CALCR polypeptide.

9. The method according to claim 1, wherein the CALCR variant nucleic acid molecule encodes a truncated CALCR polypeptide.

10. The method according to any one of claims 1 to 9, wherein the sequence analysis comprises sequencing the entire nucleic acid molecule in the biological sample.

11. The method according to any one of claims 1 to 10, wherein the sequence analysis comprises:
a) amplifying at least a portion of the nucleic acid molecule that encodes the CALCR polypeptide;
b) labeling the amplified nucleic acid molecule with a detectable label;
c) contacting the labeled nucleic acid molecule with a support comprising an alteration-specific probe; and
d) detecting the detectable label.

12. The method according to claim 11, wherein the nucleic acid molecule in the sample is mRNA and the mRNA is reverse-transcribed into cDNA prior to the amplifying step.

13. The method according to any one of claims 1 to 10, wherein the sequence analysis comprises:
contacting the nucleic acid molecule in the biological sample with an alteration-specific probe comprising a detectable label; and
detecting the detectable label.

14. The method according to any one of claims 1 to 13, wherein the nucleic acid molecule is present within a cell obtained from the subject.

15. The method according to any one of claims 1 to 14, wherein the therapeutic agent that treats or inhibits obesity and/or reduces BMI is chosen from sibutramine, orlistat, phentermine, lorcaserin, naltrexone, liraglutide, diethylpropion, bupropion, metformin, pramlintide, topiramate, and zonisamide, or any combination thereof.

16. The method according to any one of claims 1 to 14, wherein the therapeutic agent that treats or inhibits obesity and/or reduces BMI is a combination of setmelanotide and one or more of sibutramine, orlistat, phentermine, lorcaserin, naltrexone, liraglutide, diethylpropion, bupropion, metformin, pramlintide, topiramate, and zonisamide.

17. The method according to any one of claims 1 to 14, wherein the therapeutic agent that treats or inhibits obesity and/or reduces BMI is a melanocortin 4 receptor (MC4R) agonist.

18. The method according to claim 17, wherein the MC4R agonist comprises a protein, a peptide, a nucleic acid molecule, or a small molecule.

19. The method according to claim 18, wherein the protein is a peptide analog of MC4R.

20. The method according to claim 18, wherein the peptide is setmelanotide.

21. The method according to claim 18, wherein the MC4R agonist is a peptide comprising the amino acid sequence His-Phe-Arg-Trp.

22. The method according to claim 18, wherein the small molecule is 1,2,3R,4-tetrahydroisoquinoline-3-carboxylic acid.

23. The method according to claim 18, wherein the MC4R agonist is ALB-127158(a).

24. The method according to any one of claims 1 to 14, wherein the therapeutic agent that treats or inhibits obesity and/or reduces BMI is a CALCR agonist.

25. The method according to claim 24, wherein the CALCR agonist is calcitonin or amylin.

26. A method of identifying a subject having an increased risk of developing obesity and/or increased body mass index (BMI), the method comprising:
determining or having determined the presence or absence of a Calcitonin Receptor (CALCR) variant nucleic acid molecule encoding a CALCR predicted loss-of-function polypeptide in a biological sample obtained from the subject;
wherein:
when the subject is CALCR reference, the subject does not have an increased risk of developing obesity and/or increased BMI; and
when the subject is heterozygous or homozygous for a CALCR variant nucleic acid molecule, then the subject has an increased risk of developing obesity and/or increased BMI.

27. The method according to claim 26, wherein the CALCR variant nucleic acid molecule is a genomic nucleic acid molecule.

28. The method according to claim 27, wherein the determining step comprises sequencing at least a portion of the nucleotide sequence of the CALCR genomic nucleic acid molecule in the biological sample.

29. The method according to claim 25, wherein the CALCR variant nucleic acid molecule is an mRNA molecule.

30. The method according to claim 29, wherein the determining step comprises sequencing at least a portion of the nucleotide sequence of the CALCR mRNA molecule in the biological sample.

31. The method according to claim 26, wherein the CALCR variant nucleic acid molecule is a cDNA molecule produced from an mRNA molecule.

32. The method according to claim 31, wherein the determining step comprises sequencing at least a portion of the nucleotide sequence of the CALCR cDNA molecule produced from an mRNA molecule in the biological sample.

33. The method according to claim 26, wherein the CALCR variant nucleic acid molecule encoding a CALCR predicted loss-of-function polypeptide is a missense variant, a splice-site variant, a stop-gain variant, a start-loss variant, a stop-loss variant, a frameshift variant, or an in-frame indel variant, or a variant that encodes a truncated CALCR polypeptide.

34. The method according to claim 26, wherein the CALCR variant nucleic acid molecule encodes a truncated CALCR polypeptide.

35. The method according to any one of claims 26 to 34, wherein the determining step comprises sequencing the entire nucleic acid molecule in the biological sample.

36. The method according to any one of claims 26 to 35, wherein the determining step comprises:
a) amplifying at least a portion of the nucleic acid molecule that encodes the CALCR polypeptide;
b) labeling the amplified nucleic acid molecule with a detectable label;
c) contacting the labeled nucleic acid molecule with a support comprising an alteration-specific probe; and
d) detecting the detectable label.

37. The method according to claim 36, wherein the nucleic acid molecule in the sample is mRNA and the mRNA is reverse-transcribed into cDNA prior to the amplifying step.

38. The method according to any one of claims 26 to 36, wherein the determining step comprises:
contacting the nucleic acid molecule in the biological sample with an alteration-specific probe comprising a detectable label; and
detecting the detectable label.

39. The method according to any one of claims 26 to 38, wherein the nucleic acid molecule is present within a cell obtained from the subject.

40. The method according to any one of claims 26 to 39, wherein the subject is heterozygous or homozygous for the CALCR variant nucleic acid molecule, and the subject is further administered a therapeutic agent that treats or inhibits obesity and/or increased BMI.

41. The method according to claim 40, wherein the therapeutic agent that treats or inhibits obesity and/or reduces BMI is chosen from sibutramine, orlistat, phentermine, lorcaserin, naltrexone, liraglutide, diethylpropion, bupropion, metformin, pramlintide, topiramate, and zonisamide, or any combination thereof.

42. The method according to claim 41, wherein the therapeutic agent that treats or inhibits obesity and/or reduces BMI is a combination of setmelanotide and one or more of sibutramine, orlistat, phentermine, lorcaserin, naltrexone, liraglutide, diethylpropion, bupropion, metformin, pramlintide, topiramate, and zonisamide.

43. The method according to claim 40, wherein the therapeutic agent that treats or inhibits obesity and/or reduces BMI is a melanocortin 4 receptor (MC4R) agonist.

44. The method according to claim 43, wherein the MC4R agonist comprises a protein, a peptide, a nucleic acid molecule, or a small molecule.

45. The method according to claim 44, wherein the protein is a peptide analog of MC4R.

46. The method according to claim 44, wherein the peptide is setmelanotide.

47. The method according to claim 44, wherein the MC4R agonist is a peptide comprising the amino acid sequence His-Phe-Arg-Trp.

48. The method according to claim 44, wherein the small molecule is 1,2,3R,4-tetrahydroisoquinoline-3-carboxylic acid.

49. The method according to claim 44, wherein the MC4R agonist is ALB-127158(a).

50. The method according to claim 40, wherein the therapeutic agent that treats or inhibits obesity and/or reduces BMI is a CALCR agonist.

51. The method according to claim 50, wherein the CALCR agonist is calcitonin or amylin.

52. A therapeutic agent that treats or inhibits obesity and/or increased body mass index (BMI) for use in the treatment of obesity and/or increased BMI in a subject having:
a Calcitonin Receptor (CALCR) variant genomic nucleic acid molecule encoding a CALCR predicted loss-of-function polypeptide;
a CALCR variant mRNA molecule encoding a CALCR predicted loss-of-function polypeptide; or
a CALCR variant cDNA molecule encoding a CALCR predicted loss-of-function polypeptide.

53. The therapeutic agent according to claim 52, which is chosen from sibutramine, orlistat, phentermine, lorcaserin, naltrexone, liraglutide, diethylpropion, bupropion, metformin, pramlintide, topiramate, and zonisamide, or any combination thereof.

54. The therapeutic agent according to claim 53, which is a combination of setmelanotide and one or more of sibutramine, orlistat, phentermine, lorcaserin, naltrexone, liraglutide, diethylpropion, bupropion, metformin, pramlintide, topiramate, and zonisamide.

55. The therapeutic agent according to claim 52, which is a melanocortin 4 receptor (MC4R) agonist.

56. The therapeutic agent according to claim 55, wherein the MC4R agonist comprises a protein, a peptide, a nucleic acid molecule, or a small molecule.

57. The therapeutic agent according to claim 56, wherein the protein is a peptide analog of MC4R.

58. The therapeutic agent according to claim 56, wherein the peptide is setmelanotide.

59. The therapeutic agent according to claim 56, wherein the MC4R agonist is a peptide comprising the amino acid sequence His-Phe-Arg-Trp.

60. The therapeutic agent according to claim 56, wherein the small molecule is 1,2,3R,4-tetrahydroisoquinoline-3-carboxylic acid.

61. The therapeutic agent according to claim 56, wherein the MC4R agonist is ALB-127158(a).

62. The therapeutic agent according to claim 52, which is a CALCR agonist.

63. The therapeutic agent according to claim 62, wherein the CALCR agonist is calcitonin or amylin.
